(19) United States
(12) Patent Application Publication
(10) Pub. No.: US 2025/0222168 A1
(43) Pub. Date: Jul. 10, 2025

(54) METHOD OF FORMING AN ALIGNED TISSUE OR TISSUE CONSTRUCT, A TISSUE OR TISSUE CONSTRUCT, AND BIOINK

(71) Applicant: PRESIDENT AND FELLOWS OF HARVARD COLLEGE, Cambridge, MA (US)

(72) Inventors: John AHRENS, Cambridge, MA (US); Sebastian UZEL, Cambridge, MA (US); Mark Andrew SKYLAR-SCOTT, Cambridge, MA (US); Jennifer A. LEWIS, Cambridge, MA (US)

(73) Assignee: PRESIDENT AND FELLOWS OF HARVARD COLLEGE, Cambridge, MA (US)

(21) Appl. No.: 18/727,751

(22) PCT Filed: Jan. 11, 2023
(86) PCT No.: PCT/US2023/010548

§ 371 Date: Jul. 10, 2024

Related U.S. Application Data
(60) Provisional application No. 63/298,472, filed on Jan. 11, 2022.

(51) Int. Cl. A61L 27/26; A61L 27/22; A61L 27/36; A61L 27/38; A61L 27/52; B33Y 70/00; C12N 5/077

(52) U.S. Cl. CPC A61L 27/26; A61L 27/222; A61L 27/225; A61L 27/367; A61L 27/3813; A61L 27/3826; A61L 27/52; B33Y 70/00; C12N 5/0657; A61L 2430/20

(57) ABSTRACT

Described herein is a method of forming an aligned tissue or tissue construct. The method includes extruding a bioink material through a nozzle onto a support to form a structure of the bioink material, the bioink material comprising anisotropic organ building blocks (aOBBs) comprising extracellular matrix material (ECM) and cellularly aligned cells, wherein the aOBBs align parallel to the direction of the extrude path, and polymerizing the structure of the bioink material, thereby forming the tissue or tissue construct having arbitrarily programmed alignment. Also, described is a tissue or tissue construct produced by the method, as well as bioink material used to produce the same.

### CROSS-REFERENCE TO RELATED APPLICATIONS

This application claims benefit under 35 U.S.C. § 119(e) of U.S. Provisional Application No. 63/298,472 filed Jan. 11, 2022, the contents of which are incorporated herein by reference in their entirety.

### FEDERALLY SPONSORED RESEARCH OR DEVELOPMENT

This invention was made with government support under HL144043 awarded by the National Institutes of Health (NIH), and under 1647837 awarded by the National Science Foundation (NSF), and under N00014-21-1-2958 and N00014-16-1-2823 awarded by the Department of Defense/Office of Naval Research. The government has certain rights in the invention.

### BACKGROUND

The present disclosure relates generally to tissue engineering and more particularly to fabricating aligned tissue constructs.

The human heart pumps blood through a circulatory network that distributes oxygen and nutrients throughout our body. Its contractile function arises from the myocardium. Within the left ventricular myocardium, individual cardiomyocytes contract uniaxially, but their complex transmural alignment gives rise to a torsional response that enhances global ventricular function.^{1,2} In instances of heart disease or myocardial infarction, pathological remodeling can disrupt this cellular alignment and reduce contractile function.³⁻⁶ The ability to mimic the cellular composition and complex alignment of human cardiac tissue is therefore central to generating physiologically relevant tissues for drug discovery, disease modeling, and therapeutic repair.

To date, several methods have been developed to induce alignment in engineered cardiac tissues.^{[7-11]} One common approach is to seed cardiomyocytes onto micro- or nano-patterned surfaces that contain topographical cues, which guide cellular alignment.^{12,13} Another approach is to seed cells onto anisotropic polymer scaffolds¹⁴⁻¹⁶ or decellularized matrices,¹⁷ which guide tissue alignment. In addition, cell-laden hydrogels seeded into molds with varying geometries will self-assemble to produce aligned cardiac rods, rings, bundles, and sheets.^{[18-24]} Unfortunately, these methods are typically confined to thin cardiac tissues (≤100 μm thick) with either linear or radial alignment. Motivated by these limitations, extrusion-based bioprinting offers broad flexibility to control tissue composition and architecture. Recently, we and others have demonstrated that synthetic and biological fibers exhibit shear-induced alignment during printing, opening the possibility to program tissue alignment via cell templating.²⁵⁻³⁴ However, programming tissue architecture by directly aligning anisotropic human tissues has yet to be explored.

### SUMMARY

Certain embodiments relate to a method of forming a tissue or tissue construct having arbitrarily programmed alignment, comprising: extruding a bioink material through a nozzle onto a support to form a structure of the bioink material and polymerizing the structure of the bioink material, thereby forming the tissue or tissue construct having arbitrarily programmed alignment. The bioink material comprises a carrier fluid, and anisotropic organ building blocks (aOBBs), the aOBBs comprising extracellular matrix material (ECM) and cellularly aligned cells, wherein at least some of the aOBBs align along a direction of extrusion in the structure of the bioink material. In the method, the aOBBs may comprise functional cells, stromal cells, or a mixture of functional and stromal cells. In the method, the aOBBs may comprise cardiomyocytes. In the method, the aOBBs may comprise contractile induced pluripotent stem cell derived cardiomyocytes (iPSC-CMs) and stromal, human neonatal dermal fibroblasts (hNDF). The aOBBs may comprise primary cardiomyocytes or embryonic stem cell derived cardiomyocytes (ESC-CM). Cardiomyocytes are iPSC-CM, but they could also be primary or ESC-CM. In the method, the aOBBs may further comprise support cells selected from the group consisting of primary fibroblasts, mesenchymal stem cells, epicardial derived cells, and a combination thereof. In the method, the aOBBs may comprise contractile skeletal muscle cells (either primary or stem cell derived), mesenchymal stem cells (as stromal cells), neurons, endothelial cells, smooth-muscle cells, and fibroblasts. The ECM may comprise a blend of collagen and fibrin. Alternatively, the ECM may comprise any combination of biological polymers (collagen, fibrin, matrigel, hyaluomic acid, silk, alginate, chitosan) or synthetic materials (polyglycolic acid (PGA), poly(L)-lactic acid (PLA), poly(DL) glycolate (PLGA), and polyvinyl alcohol and their derivatives). The method may further comprise providing the bioink material. The step of providing the bioink material comprises: culturing the aOBBs within a pillar array; harvesting the aOBBs from the pillar array by manual pipetting or by degradation of the pillar array; resuspending the aOBBs in the carrier fluid; compacting the aOBBs to achieve high volume fractions (i.e., cellular densities) to form the bioink material; and adjusting rheology of the bioink material by modulating temperature. The carrier fluid may comprise a processed gelatin, fibrinogen and cell media. The cellular density within the bioink may be 10-500e6 cell/mL. The support may be a glass, plastic, or a biological hydrogel or matrix. The cellular alignment may form a tissue pattern(s) selected from the group consisting of linear, chevron, spiral pattern, and a combination of patterns. The method may further comprise vascularizing the tissue or tissue construct.

Another embodiment relates to a tissue or tissue construct prepared by the described method, the tissue or tissue construct aligned at the aOBB, cellular, and sarcomeric length scales. In the tissue or tissue construct the aOBBS may have isotropic cellular alignment. Alternatively, the aOBBs may have anisotropic cellular alignment.

Another embodiment relates to the use of a tissue or tissue construct produced by the methods described herein in disease modeling.

Another embodiment relates to the use of a tissue or tissue construct produced by the methods described herein in cardiac disease modeling.

Another embodiment relates to the use of a tissue or tissue construct produced by the methods described herein in drug toxicity studies.

Another embodiment relates to the use of a tissue or tissue construct produced by the methods described herein in drug screening applications.

Another embodiment relates to the use of a tissue or tissue construct produced by the methods described herein as cardiac tissue for replacement of heart in regenerative medicine.

A further embodiment relates to a bioink material for use with a three-dimensional printer or an additive manufacturing system, the bioink comprising: a carrier fluid, and anisotropic organ building blocks (aOBBs) comprising extracellular matrix material (ECM) and cellularly aligned cells, wherein at least some of the aOBBs are capable of anisotropically align along an extrusion direction when the bioink is extruded from a three-dimensional printer or additive manufacturing system. In the bioink, the cellular density within the bioink may be 10-500e6 cell/mL.

### BRIEF DESCRIPTION OF THE DRAWINGS

FIGS. 1A-1C depict aligned cardiac tissues via bioprinting anisotropic organ building blocks. (FIG. 1A) Image sequence highlighting the key steps in cardiac bioink preparation: Step (i) iPSC-derived cardiac spheroids are generated and then dissociated into hiPSC-CMs, (ii) hiPSC-CMs are combined with hNDFs in a 9:1 ratio, suspended in collagen-based gel, and seeded into micro-pillar arrays, (iii) the cells self-assemble into anisotropic organ building blocks (aOBBs), (iv-v) aOBBs are then harvested and compacted in a syringe. [Scale bars: (i): 200 μm, (ii-iii): 500 μm, (iii-iv): 1000 μm.](FIG. 1B) Schematic illustration of aOBB orientation within the compacted bioink, which depicts their initial random orientation in the syringe reservoir followed by their shear-induced alignment during ink extrusion from nozzle. (FIG. 1C) Multiscale alignment generated via bioprinting cardiac inks composed of aOBBs where (i) sarcomere alignment is observed within each cell, (ii) individual cells are aligned within aOBBs, and (iii) aOBBs are aligned within a bioprinted tissue. [Scale bars, i: 20 μm, ii: 100 μm, iii: 2 mm.]

FIGS. 2A-2O depict programming complex alignment in model and cardiac tissues. (FIG. 2A) Visualization of pre-labelled anisotropic organ building blocks (aOBBs) printed at 5 mm s⁻¹ and 3D volumetric models. (FIG. 2B) Polar histogram of aOBB orientations. (FIG. 2C) Quantification of the spread of aOBB orientation as a function of print speed (1-10 mm s⁻¹). (FIGS. 2D-2F) 3D bioprinted, hNDF-based model sheets with varying patterns. Scale Bars: 2000 μm. (FIGS. 2G-2I) 3D printed, cardiac-based sheets with varying patterns. Scale Bars: 2000 μm. Polar histograms for aOBB orientation in (FIGS. 2J, 2K) vertical, (FIGS. 2L, 2M) chevron, and (FIGS. 2N, 2O) circular patterned sheets. Top row (FIGS. 2J, 2L, 2N) and bottom row (FIGS. 2K, 2M, 2O) corresponds to hNDF and cardiac macro-tissue sheets, respectively. All yellow bars indicate resultant vector lengths.

FIGS. 3A-3G depict aligned cardiac macro-tissues. (FIG. 3A) Schematic overview of the bioprinting process used to generate aligned cardiac macro-tissues on macro-pillars. (FIG. 3B) Visualization of cellular alignment within bioprinted, anisotropic organ building block (aOBB)-based, cardiac macro-tissues: (i) Day 0, 5×. (ii) tissue surface, Day 0, 20×, (iii) tissue surface, Day 7, 20×, and (iv) tissue core, Day 7, 20×. Scale bars (i):1000 μm, (ii-iv): 100 μm. (FIG. 3C) Visualization of cellular alignment within bioprinted spheroid-based, cardiac macro-tissues: (i) Day 0, 5×, (ii) tissue surface, Day 0, 20×, (iii) tissue surface, Day 7, 20×, (iv) tissue core, Day 7, 20×. Scale bars (i):1000 μm, (ii-iv): 100 μm. (FIGS. 3D-3F) Polar histograms display primary orientations extracted from high magnification (20×) images of anisotropic and spheroid-based cardiac macro-tissues. Light bars represent resultant vectors. (FIG. 3G) Resultant vectors indicate distribution of polar histogram data from 20× images; p***<0.001.

FIGS. 4A-4H depict functional characterization and maturation of aligned cardiac macro-tissues. Activation maps derived from calcium imaging of bioprinted cardiac macro-tissues composed of aniostropic and spheroid-based organ building blocks (OBBs) at (FIG. 4A) Day 0, and (FIG. 4B) Day 4. (FIG. 4C) Conduction velocity measurements derived from activation maps of these bioprinted tissues. (FIG. 4D) Force measurements of bioprinted tissues over time; p*<0.05, p**<0.01. (FIG. 4E) Force normalized per cardiomyocyte; p**<0.01, p***<0.001. (FIG. 4F) Force on Day 7 normalized by cross-sectional area; p**<0.01. (FIG. 4G) Electrical stimulation of the bioprinted tissues (1-3 Hz). (FIG. 4H) Force-frequency relationship for electrically stimulated, bioprinted tissues.

FIG. 5 depicts micro-pillar arrays for scalable generation of aOBBs. (left) Micro-pillar arrays are first produced by stereolithography (SLA). They are UV cured and surface treated to covalently attach fluorosilane to prevent inhibition of silicone polymerization. (middle) After treatment, the micro-pillar arrays are slotted into custom acrylic cutout holders. SortaClear37™, a tough, flexible silicone is used to generate a negative plate. To facilitate silicone-on-silicone transfer molding, fluorosilane is applied the surface of the SortaClear37™. (right) A silicone resin is poured onto the mold and cured to generate a contiguous PDMS plate of micro-pillar arrays. Scale bars: 1000 μm.

FIGS. 6A-6B depict cardiac spheroid differentiation efficiency. Flow cytometry gating strategy is selected as follows: (top row) cells gate (FSC-A vs. SSC-A), (second row) single cells gate (SSC-A Vs SSC-W), (third row) exclusion of dead cells (FSC Vs. Live/Dead) and (fourth row) final quadrant gate showing cardiac lineage markers Cardiac Troponin T (cTnT) versus Vimentin expression. All dot plots represent the percent of parent gate.

FIG. 7 depicts efficient and scalable seeding of anisotropic organ building blocks (aOBBs). Plasma treatment temporarily increases the hydrophilicity of the PDMS surface, which enables increased wetting of the collagen-cell mixture. A glass slide is used to distribute and wick the cell-gel mixture into all microwells evenly. After polymerization at 37° C. for 25 min, media is added to the tissues.

FIGS. 8A-8G depict scalable generation of cellularly aligned, anisotropic organ building blocks (OBBs). (FIG. 8A) Immunofluorescent staining of an individual aOBB demonstrates aligned sarcomeres by Day 3. Scale bars, top: 200 μm, bottom: 20 μm. (FIG. 8B) Fourier analysis used to quantify the high degree of sarcomere alignment. (FIG. 8C) Without capped ends, aOBBs slide off micro-pillars overtime. (FIG. 8D) aOBB force generation on Day 3. n=5 separate passages. (FIG. 8E) ROCK inhibitor (Y-27632) temporarily blunts aOBB contraction. Full contractile function is recovered after washing. n=10 aOBBs from 1 passage. (FIGS. 8F, 8G) Pre-incubation of aOBBs in ROCK inhibitor (Y-27632) blunts compaction upon harvest from micropillar array. Scale bars, 1000 μm.

FIG. 9 depicts a two-part mold for bioprinting cardiac macro-tissues. The stability ledge and pillar cap stabilize the z-height of the printed tissue. Stereolithography is used to manufacture both Part A and Part B. Accurate joining of Part A and Part B was enabled by slip fit connections. M4 screws passed through the compression holes and ensured a tight, leak-free joining of Part A and Part B.

FIG. 10 depicts a macro-pillar platform for bioprinting. High-walled polycarbonate plate was milled to snugly fit the silicone printing platforms. After assembly is autoclaved, a flush layer of sacrificial gelatin is cast to sit flush with macro-pillar ledge. Top row: schematics. Bottom row: photos.

FIGS. 11A-11C depict measured cell density in cryo-sectioned slices of bioprinted cardiac macro-tissues. (FIG. 11A) Comparison of cellular densities between (control) spheroid- and aOBB-laden bioprinted cardiac macro-tissues on Day 0, where n=5 tissues from 3 separate experiments. Representative high magnification images of nuclear stained 15 μm cryo-sectioned slices from (FIG. 11B) spheroid-laden, and (FIG. 11C) aOBB-laden cardiac macro-tissues. Nuclear counting was performed by Imaris spot tracking. n=5 imaging windows per tissue. Scale bars, 10 μm.

FIGS. 12A-12F depict cellular alignment within core of bioprinted cardiac macro-tissues. High magnification confocal images of cryo-sectioned slices from (FIGS. 12A-12C) control (spheroid) and (FIGS. 12D-12F) aOBB-based bioprinted cardiac macro-tissues overtime. Scale bars, 100 μm.

FIGS. 13A-13B depict cardiac macro-tissue displacement. Measured displacement for control (spheroid) and aOBB-based bioprinted cardiac macro-tissues at Day 1 and Day 3. Note, the building blocks fuse into synchronously contractile tissue over time, as demonstrated by a decrease in the standard deviation of displacement values observed from Day 1 to Day 3.

FIGS. 14A-14C depict contractile force measurements for aligned cardiac tissues. Force measurements are quantified using Euler-Bernoulli beam theory. Image and schematic representations of (FIG. 14A) micro- and (FIG. 14B) macro-pillars were quantified using an Instron. Micro- and macro-pillar dimensions were measured from high resolution images recorded across 20 independent samples. (FIG. 14C) The material and geometric information were combined to numerically estimate cantilever spring constants and translate pillar displacement to force generation.

FIGS. 15A-15C depict the cellular composition of bioprinted cardiac macro-tissues. Immediately after printing, individual filaments were collected for analysis prior to polymerization. Their cellular composition is quantified using flow cytometry for cell-specific markers. (FIG. 15A) Live cells were gated based on nuclear DAPI stain; *p<0.05. (FIG. 15B) iPSC-CMs were gated based on cTnT; *p<0.05. (FIG. 15C) hNDFs were gated based on vimentin. n=5, from two separate passages.

FIGS. 16A-16C depict cell viability in aligned cardiac macro-tissues. Measured cell viability after dissociation of bioprinted aOBB-based cardiac macro-tissues on (FIG. 16A) Day 0; n=5 and (FIG. 16B) Day 7; n=1. (FIG. 16C) Cross-sectional slices of bioprinted cardiac macro-tissues on Day 7. Each slice is from a different macro-tissue, each from a distinct experiment. Live cells are stained via cytoplasmic marker Calcein-AM while dead cells are stained via nuclear marker Ethidium 1 homodimer (Ethd-1). Scale Bars, 200 μm.

### DETAILED DESCRIPTION

The ability to replicate the three-dimensional myocardial architecture found in human hearts is a grand challenge. Here, the fabrication of aligned cardiac tissue by bioprinting anisotropic organ building blocks composed of human induced pluripotent stem cell-derived cardiomyocytes (hiPSC-CMs) is described. First, a bioink that contains cellularly aligned, contractile cardiac micro-tissues was generated. Second, extrusion-based bioprinting was used to orient these anisotropic organ building blocks along the printing direction. Next, their alignment within printed cardiac tissue sheets patterned with linear, spiral, and chevron features was programmably controlled. Finally, aligned cardiac macro-tissues were generated, and their contractile force and conduction velocity was shown, which increases over time, exhibit higher values than control tissues formed by spherical organ building blocks. The described invention and supporting data open new avenues to generating functional cardiac tissue with high cell density and complex cellularly alignment.

Described herein is a novel method to fabricate stem-cell derived, tissue or tissue construct at high cellular density with programmable alignment. Although the embodiments described herein refer to methods of preparing a cardiac tissue, the presently described method may be utilized to produce other, highly structured tissue or tissue constructs with complex patterns.

In one aspect of any of the embodiments, described herein is a method comprising applying a bioink material onto a support, the bioink material comprising:1) a carrier fluid, and 2) a plurality of anisotropic organ building blocks (aOBBs), each of the plurality of aOBBs comprising: at least one polymer and at least one cell; wherein the applied bioink material comprises a plurality of aligned aOBBs.

As used herein, “anisotropic” refers to the property of a three-dimensional object having a greater size along a first dimensional axis than in the two dimensional axes that are perpendicular to the first dimensional axis. That is, for a three-dimensional axis in which one dimension is designated as its length, it has a length which is larger than its width and its height. The width and height can be equal or nonequal in size relative to each other. The axis of the largest dimension of an anisotropic object, e.g., the axis defined by its largest cross-sectional measurement is referred to herein as the object's “anisotropic axis.”

As used herein, “anisotropic organ building block” or “aOBB” refers to three-dimensional object comprising at least one cell and at least one polymer, where the object is anisotropic. An aOBB can have any three-dimensional shape, e.g., rectangular, filamentous, ovoid, etc. In some embodiments of any of the aspects, the aOBB has a rectangular shape. In some embodiments of any of the aspects, an aOBB comprises one polymer. In some embodiments of any of the aspects, an aOBB comprises two polymers. In some embodiments of any of the aspects, an aOBB comprises three polymers.

As used herein, the term “polymer” refers to oligomers, co-oligomers, polymers and co-polymers, e.g., random block, multiblock, star, grafted, gradient copolymers and combination thereof. The average molecular weight of the polymer, as determined by gel permeation chromatography, can range from 500 to about 500,000, e.g., from 20,000 to about 500,000. Without limitation, any polymeric material known in the art can be used in the invention. Accordingly, in some embodiments, the polymer is selected from the group consisting of polysaccharides, polypeptides, polynucleotides, copolymers of fumaric/sebacic acid, poloxamers, polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), gelatin, collagen, silk, alginate, cellulose, poly-nucleic acids, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), hyaluron, heparin, agarose, pullulan, and copolymers, terpolymers, and copolymers comprising any combinations thereof.

In some embodiments of any of the aspects, the polymer is a biomaterial. In some embodiments of any of the aspects, the at least one polymer comprises at least one biomaterial. As used herein, the term “biomaterial” refers to a material which is naturally-occurring in an organism. Examples of such biomaterial polymers are known in the art and provided herein, e.g., the extracellular matrix materials described herein. By way of non-limiting example, the at least one polymer can comprise one or more of: collagen, fibrin, MATRIGEL, hyaluronic acid, silk, alginate, chitosan, alginate, gelatin, heparin, chondroitin sulfate, polyethylene glycol (PEG), polyethylene glycol diacrylate (PEGDA), polyglycolic acid (PGA), poly(L)-lactic acid (PLA), poly(DL) glycolate (PLGA), and polyvinyl alcohol and their derivatives. In some embodiments of any of the aspects, the at least one polymer can comprise one or more of: collagen, fibrin, MATRIGEL, hyaluronic acid, silk, alginate, chitosan, alginate, gelatin, heparin, chondroitin sulfate, polyethylene glycol (PEG), polyethylene glycol diacrylate (PEGDA), polyglycolic acid (PGA), poly(L)-lactic acid (PLA), poly(DL) glycolate (PLGA), and polyvinyl alcohol.

In some embodiments of any of the aspects, the at least one polymer can comprise one or more of: fibrin, hyaluronic acid, alginate, heparin, chondroitin sulfate, polyethylene glycol (PEG), and polyethylene glycol diacrylate (PEGDA). In some embodiments of any of the aspects, the at least one polymer can comprise two or more of: fibrin, hyaluronic acid, alginate, heparin, chondroitin sulfate, polyethylene glycol (PEG), and polyethylene glycol diacrylate (PEGDA). In some embodiments of any of the aspects, the at least one polymer can comprise three or more of: fibrin, hyaluronic acid, alginate, heparin, chondroitin sulfate, polyethylene glycol (PEG), and polyethylene glycol diacrylate (PEGDA).

In some embodiments of any of the aspects, the at least one polymer consists of one of: fibrin, hyaluronic acid, alginate, heparin, chondroitin sulfate, polyethylene glycol (PEG), and polyethylene glycol diacrylate (PEGDA). In some embodiments of any of the aspects, the at least one polymer consists of two of: fibrin, hyaluronic acid, alginate, heparin, chondroitin sulfate, polyethylene glycol (PEG), and polyethylene glycol diacrylate (PEGDA). In some embodiments of any of the aspects, the at least one polymer consists of three of: fibrin, hyaluronic acid, alginate, heparin, chondroitin sulfate, polyethylene glycol (PEG), and polyethylene glycol diacrylate (PEGDA).

In some embodiments of any of the aspects, the at least one polymer comprises collagen. In some embodiments of any of the aspects, the at least one polymer consists of collagen. In some embodiments of any of the aspects, the at least one polymer comprises fibrin. In some embodiments of any of the aspects, the at least one polymer consists of fibrin. In some embodiments of any of the aspects, the at least one polymer comprises collagen and fibrin. In some embodiments of any of the aspects, the at least one polymer consists of collagen and fibrin.

In some embodiments of any of the aspects, the at least one polymer comprises alginate. In some embodiments of any of the aspects, the at least one polymer consists of alginate.

In some embodiments of any of the aspects, the at least one polymer comprises alginate and PEG. In some embodiments of any of the aspects, the at least one polymer consists of alginate and PEG.

In some embodiments of any of the aspects, the at least one polymer comprises alginate and PEGDA. In some embodiments of any of the aspects, the at least one polymer consists of alginate and PEGDA.

In some embodiments of any of the aspects, the at least one polymer comprises gelatin and alginate. In some embodiments of any of the aspects, the at least one polymer consists of gelatin and alginate.

In some embodiments of any of the aspects, the at least one polymer comprises gelatin and collagen.In some embodiments of any of the aspects, the at least one polymer consists of gelatin and collagen.

In some embodiments of any of the aspects, the at least one polymer comprises gelatin and fibrin.In some embodiments of any of the aspects, the at least one polymer consists of gelatin and fibrin.

In some embodiments of any of the aspects the at least one polymer can be modified with motifs or moieties. For example, the at least one polymer can further comprise RGD cell binding sequences, e.g., alginate can be modified to comprise one or more RGD peptides.

In some embodiments, the polymer is a biocompatible polymer. As used herein, the term “biocompatible” means exhibition of essentially no cytotoxicity or immunogenicity while in contact with body fluids or tissues. The term “biocompatible polymer” refers to polymers which are non-toxic, chemically inert, and substantially non-immunogenic when used internally in a subject and which are substantially insoluble in blood. The biocompatible polymer can be either non-biodegradable or preferably biodegradable. Preferably, the biocompatible polymer is also non-inflammatory when employed in situ.

Biodegradable polymers are disclosed in the art. Examples of suitable biodegradable polymers include, but are not limited to, linear-chain polymers such as polypeptides, polynucleotides, polysaccharides, polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), fumaric acid, sebacic acid, and copolymers, terpolymers including one or more of the foregoing. Other biodegradable polymers include, for example, gelatin, collagen, silk, chitosan, alginate, cellulose, poly-nucleic acids, etc.

Suitable non-biodegradable biocompatible polymers include, by way of example, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), Poloxamers (e.g., Pluronic such as Poloxamers 407 and 188), hyaluronic acid, heparin, agarose, Pullulan, and copolymers including one or more of the foregoing, such as ethylene/vinyl alcohol copolymers (EVOH).

In some embodiments, the polymer is a homopolymer, a copolymer or a block polymer.

In some embodiments, the polymer comprises side chains selected from the group consisting of amide or ester groups. In some embodiments, the polymer is biodegradable, biocompatible, and non-toxic.

The polymer can be derivatized with a second polymer and the first polymer and the second polymer can be the same or different. For example, the polymer can be derivatized with a polyethylene glycol (PEG).

In some embodiments, polymers or portions of polymers can be connected by linkers. In some embodiments, components of a polymeric particle, e.g., a payload reagent or monocyte-targeting and/or macrophage-targeting ligand can be connected via a linker. As used herein, the term “linker” refers to a moiety that connects two parts of a compound. Linkers typically comprise a direct bond or an atom such as oxygen or sulfur, a unit such as NR₁, C(O), C(O)O, C(O)NR₁, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R₁)₂, C(O), cleavable linking group, substituted or unsubstituted aryl, substituted or unsubstituted heteroaryl, substituted or unsubstituted heterocyclic; where R₁ is hydrogen, acyl, aliphatic or substituted aliphatic.

The linker can be a branched linker. The branch-point of the branched linker can be at least divalent, but can be a trivalent, tetravalent, pentavalent or hexavalent atom, or a group presenting such multiple valencies. In certain embodiments, the branch-point can be, —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branch-point can be an acrylate, cyanoacrylate, or methylacrylate.

In various embodiments, the linker is a cleavable linker. A cleavable linker means that the linker can be cleaved to release the two parts the linker is holding together. A cleavable linker can be susceptible to cleavage agents, such as, but not limited to, enzymes, pH, redox potential or the presence of degradative molecules. Examples of such agents: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

In some embodiments, the cleavable linker is cleavable by an enzyme. In some embodiments, the cleavable linker is selected from a group consisting of small molecules. In some preferred embodiments, the cleavable linker is selected from a group consisting of peptides or polypeptides.

In some embodiments of any of the aspects, the at least one polymer comprises at least one extracellular matrix material. Examples of extracellular matrix materials include but are not limited to collagen, fibronectin, fibrinogen, poly-lysine, vitronectin, laminin, elastin, tenascin, and Matrigel®. Other extracellular matrix formulations and proteins are known in the art. In some embodiments of any of the aspects, the extracellular matrix material comprises a gelatinous protein mixture secreted by Engelbreth-Holm-Swarm (EHS) mouse sarcoma cells. In some embodiments, the ECM composition comprises one or more of laminin, Collagen IV, heparan sulfate proteoglycans, entactin/nidogen, fibronectin, or any combination thereof. In some embodiments of any of the aspects, the gelatinous protein mixture secreted by Engelbreth-Holm-Swarm (EHS) mouse sarcoma cells is MATRIGEL™. MATRIGEL™ is commercially available, for example, from CORNING® [MATRIGEL® Matrix (Catalog #356231)]. In certain embodiments, the ECM may be or may include at least one of Matrigel®, poly L-lysine, Geltrex™, gelatin, nitrogen, fibronectin, collagen 1, collagen IV, fibrinogen, gelatin methacrylate, fibrin, silk, pegylated gels, collagen methacrylate, basement membrane proteins, or any other biomaterial, or a combination thereof. The extracellular matrix material or composition of aOBBs can include a blend of collagen and fibrin. Other ECM components could be included or substituted, such as hyaluronic acid and MATRIGEL.

In some embodiments of any of the aspects, the at least one polymer comprises at least one synthetic polymer. As used herein, the term “synthetic polymer” refers to a material which is not naturally-occurring in an organism.

In some embodiments of any of the aspects, an aOBB has a minimum anisotropy ration of 2:1. In some embodiments of any of the aspects, an aOBB has a minimum anisotropy ration of 1.5:1. In some embodiments of any of the aspects, an aOBB has a minimum anisotropy ration of 2.5:1. In some embodiments of any of the aspects, an aOBB has a minimum anisotropy ration of 3:1. In some embodiments of any of the aspects, an aOBB has a minimum anisotropy ration of 5:1. In some embodiments of any of the aspects, an aOBB has a minimum anisotropy ration of 10:1.

In some embodiments of any of the aspects, an aOBB has a cross-section diameter of from about 10 μm to about 2000 μm. In some embodiments of any of the aspects, an aOBB has a cross-section diameter of from 10 μm to 2000 μm. In some embodiments of any of the aspects, an aOBB has a cross-section diameter of from about 50 μm to about 1000 μm. In some embodiments of any of the aspects, an aOBB has a cross-section diameter of from 50 μm to 1000 μm.

In some embodiments of any of the aspects, an aOBB comprises a single cell type. In some embodiments of any of the aspects, an aOBB comprises two cell types. In some embodiments of any of the aspects, an aOBB comprises three cell types. In some embodiments of any of the aspects, an aOBB comprises four cell types. In some embodiments of any of the aspects, an aOBB comprises five or more cell types.

In some embodiments of any of the aspects, the at least one cell comprises a plurality of cells. In some embodiments of any of the aspects, the at least one cell comprises a plurality of different cell types.

In some embodiments of any of the aspects, the at least one cell comprises at least one functional cell. In some embodiments of any of the aspects, the at least one cell comprises at least one stromal cell. In some embodiments of any of the aspects, the at least one cell comprises at least one functional cell and at least one stromal cell.

As used herein, “stromal cell” refers to non-vascular, non-inflammatory, non-epithelial connective tissue cells of that surround an organ or non-connective tissue. Stromal cells support the function of the parenchymal cells of that organ. Fibroblasts and pericytes are among the most common types of stromal cells. The stromal cells can be derived from numerous body tissue types, including, but not limited to, breast tissue, thymic tissue, bone marrow tissue, bone tissue, dermal tissue, muscle tissue, respiratory tract tissue, gastrointestinal tract tissue, genitourinary tissue, central nervous system tissue, peripheral nervous system tissue, reproductive tract tissue. In an embodiment, stromal cells include mesenchymal stromal cells (MSC).

In some embodiments of any of the aspects, the at least one stromal cell comprises at least one fibroblast. In some embodiments of any of the aspects, the at least one stromal cell consists of at least one fibroblast. In some embodiments, a fibroblast is a human neonatal dermal fibroblast (hNDF).

In some embodiments of any of the aspects, the at least one stromal cell comprises at least one MSC. In some embodiments of any of the aspects, the at least one stromal cell consists of at least one MSC.

In some embodiments, the at least one cell comprises one or more support cells selected from the group consisting of: primary fibroblasts, mesenchymal stem cells, epicardial derived cells, and a combination thereof. In some embodiments, the at least one stromal cell comprises one or more support cells selected from the group consisting of: primary fibroblasts, mesenchymal stem cells, epicardial derived cells, and a combination thereof.

As used herein, “functional cell”or “parenchymal cell” refers to a cell type that contributes to the function (as opposed to the maintenance or structural support) of an organ. Functional cells are well known and include, without limitation, neurons, glial cells, hepatocytes, myocytes, cardiomyoctes, and the like. Myoctes can comprise induced pluripotent stem cell derived myocytes, primary myocytes, and/or or embryonic stem cell derived myocytes. Myocytes can comprise cardiomyoctes and/or smooth muscle cells. In some embodiments at least one myocyte comprises at least one smooth muscle cell. In some embodiments at least one myocyte consists of at least one smooth muscle cell. Cardiomyoctes can comprise contractile induced pluripotent stem cell derived cardiomyocytes (iPSC-CMs), primary cardiomyocytes, and/or or embryonic stem cell derived cardiomyocytes (ESC-CM).

In some embodiments of any of the aspects, the functional cell comprises myocytes. In some embodiments of any of the aspects, the functional cell consists of myocytes. In some embodiments of any of the aspects, the functional cell comprises cardiomyocytes. In some embodiments of any of the aspects, the functional cell consists of cardiomyocytes.

In some embodiments of any of the aspects, the at least one cell comprises cardiomyocytes and stromal cells. In some embodiments of any of the aspects, the at least one cell comprises cardiomyocytes and fibroblasts. In some embodiments of any of the aspects, the at least one cell consists of cardiomyocytes and stromal cells. In some embodiments of any of the aspects, the at least one cell consists of cardiomyocytes and fibroblasts.

In some embodiments of any of the aspects, the at least one cell comprises myocytes and stromal cells. In some embodiments of any of the aspects, the at least one cell comprises myocytes and fibroblasts. In some embodiments of any of the aspects, the at least one cell consists of myocytes and stromal cells. In some embodiments of any of the aspects, the at least one cell consists of myocytes and fibroblasts.

In some embodiments of any of the aspects, the at least one cell comprises one or more of: contractile skeletal muscle cells, skeletal muscle precursor cells (either primary or stem cell derived), skeletal muscle cells (either primary or stem cell derived), mesenchymal stem cells (e.g., stromal cells), neurons, endothelial cells, adipocytes, smooth-muscle cells, and fibroblasts. In some embodiments of any of the aspects, the at least one cell comprises: skeletal muscle cells and adipocytes. In some embodiments of any of the aspects, the at least one cell comprises skeletal muscle cells and fibroblasts. In some embodiments of any of the aspects, the at least one cell comprises skeletal muscle cells and endothelial cells. In some embodiments of any of the aspects, the at least one cell comprises skeletal muscle cells, adipocytes, and endothelial cells.

The cellular composition of aOBBs is arbitrary and could contain 100% functional, 100% stromal, or a mixture of functional and stromal cells. In a cardiac context, aOBBs can contain contractile induced pluripotent stem cell derived cardiomyocytes (iPSC-CMs) and stromal, human neonatal dermal fibroblasts (hNDF). Contractile cells could also be primary cardiomyocytes or embryonic stem cell derived cardiomyocytes (ESC-CM). Other support cells could include primary fibroblasts, mesenchymal stem cells, and/or epicardial derived cells. Within a musculoskeletal context, aOBBS could contain contractile skeletal muscle cells, either primary or stem cell derived, and mesenchymal stem cells (MSC) as stromal cells. Tendon-like tissue constructs could be generated using aOBBs containing 100% stromal cells such as MSCs. Within a neuronal context, aOBBs could contain neurons. Within a vascular context, aOBBs could contain endothelial cells, smooth-muscle cells, and fibroblasts.

In some embodiments of any of the aspects, a plurality of cells of a single aOBB can be arranged in any manner with respect to each other. In some embodiments of any of the aspects, a plurality of cells of a single aOBB can be randomly arranged in any manner with respect to each other. In some embodiments of any of the aspects, a plurality of cells of a single aOBB can be arranged in any manner with respect to the anisotropy of the aOBB. In some embodiments of any of the aspects, a plurality of cells of a single aOBB can be randomly arranged in any manner with respect to the anisotropy of the aOBB.

Alternatively, a plurality of cells of a single aOBB can be cellularly aligned. As used herein, “cellularly aligned” refers to a plurality of cells which are themselves anisotropic and which display some degree of alignment of their individual anisotropic axes, e.g., a degree of alignment which is statistically not random. Methods of preparing such cellularly aligned aOBBs are provided elsewhere herein.

In some embodiments of any of the aspects, at least 90% of the plurality of cells have a cellular anisotropic axis that is within +/−70 degrees of a mean cellular anisotropic axis of the plurality of cells of the aOBB. In some embodiments of any of the aspects, at least 90% of the plurality of cells have a cellular anisotropic axis that is within +/−60 degrees of a mean cellular anisotropic axis of the plurality of cells of the aOBB. In some embodiments of any of the aspects, at least 90% of the plurality of cells have a cellular anisotropic axis that is within +/−50 degrees of a mean cellular anisotropic axis of the plurality of cells of the aOBB. In some embodiments of any of the aspects, at least 90% of the plurality of cells have a cellular anisotropic axis that is within +/−40 degrees of a mean cellular anisotropic axis of the plurality of cells of the aOBB. In some embodiments of any of the aspects, at least 90% of the plurality of cells have a cellular anisotropic axis that is within +/−30 degrees of a mean cellular anisotropic axis of the plurality of cells of the aOBB. In some embodiments of any of the aspects, at least 90% of the plurality of cells have a cellular anisotropic axis that is within +/−20 degrees of a mean cellular anisotropic axis of the plurality of cells of the aOBB. In some embodiments of any of the aspects, at least 90% of the plurality of cells have a cellular anisotropic axis that is within +/−10 degrees of a mean cellular anisotropic axis of the plurality of cells of the aOBB.

In some embodiments of any of the aspects, at least 60% of the plurality of cells have a cellular anisotropic axis that is within +/−40 degrees of a mean cellular anisotropic axis of the plurality of cells of the aOBB. In some embodiments of any of the aspects, at least 60% of the plurality of cells have a cellular anisotropic axis that is within +/−30 degrees of a mean cellular anisotropic axis of the plurality of cells of the aOBB. In some embodiments of any of the aspects, at least 60% of the plurality of cells have a cellular anisotropic axis that is within +/−20 degrees of a mean cellular anisotropic axis of the plurality of cells of the aOBB. In some embodiments of any of the aspects, at least 60% of the plurality of cells have a cellular anisotropic axis that is within +/−10 degrees of a mean cellular anisotropic axis of the plurality of cells of the aOBB.

In some embodiments of any of the aspect, the degrees are as measured in one axis perpendicular to the mean cellular anisotropic axis of the plurality of cells of the aOBB. In some embodiments of any of the aspect, the degrees are as measured in both axes perpendicular to the mean cellular anisotropic axis of the plurality of cells of the aOBB. In some embodiments of any of the aspect, the a cells have a cellular anisotropic axis within a certain number of degrees of a mean cellular anisotropic axis of the plurality of cells of the aOBB in one axis perpendicular to the mean cellular anisotropic axis of the plurality of cells of the aOBB (e.g., if the mean cellular anisotropic axis of the plurality of cells of the aOBB is the x axis, the degrees are in the y or the z axis). In some embodiments of any of the aspect, the cells have a cellular anisotropic axis within a certain number of degrees of a mean cellular anisotropic axis of the plurality of cells of the aOBB in the first axis perpendicular to the mean cellular anisotropic axis of the plurality of cells of the aOBB and within that same number of degrees of the mean cellular anisotropic axis of the plurality of cells of the aOBB in the second axis that is perpendicular to the mean cellular anisotropic axis of the plurality of cells of the aOBB (e.g., if the mean cellular anisotropic axis of the plurality of cells of the aOBB is the x axis, the degrees are in the y axis and also in the z axis).

In some embodiments of any of the aspects, at least 50% of the plurality of cells have a cellular anisotropic axis that is within 1 standard deviation of a mean cellular anisotropic axis of the plurality of cells of the aOBB. In some embodiments of any of the aspects, at least 60% of the plurality of cells have a cellular anisotropic axis that is within 1 standard deviation of a mean cellular anisotropic axis of the plurality of cells of the aOBB. In some embodiments of any of the aspects, at least 70% of the plurality of cells have a cellular anisotropic axis that is within 1 standard deviation of a mean cellular anisotropic axis of the plurality of cells of the aOBB. In some embodiments of any of the aspects, at least 80% of the plurality of cells have a cellular anisotropic axis that is within 1 standard deviation of a mean cellular anisotropic axis of the plurality of cells of the aOBB. In some embodiments of any of the aspects, at least 90% of the plurality of cells have a cellular anisotropic axis that is within 1 standard deviation of a mean cellular anisotropic axis of the plurality of cells of the aOBB.

In some embodiments of any of the aspects, at least 80% of the plurality of cells have a cellular anisotropic axis that is within 2 standard deviations of a mean cellular anisotropic axis of the plurality of cells of the aOBB. In some embodiments of any of the aspects, at least 90% of the plurality of cells have a cellular anisotropic axis that is within 2 standard deviations of a mean cellular anisotropic axis of the plurality of cells of the aOBB. In some embodiments of any of the aspects, at least 95% of the plurality of cells have a cellular anisotropic axis that is within 2 standard deviations of a mean cellular anisotropic axis of the plurality of cells of the aOBB.

In some embodiments of any of the aspect, the deviation is as measured in one axis perpendicular to the mean cellular anisotropic axis of the plurality of cells of the aOBB. In some embodiments of any of the aspect, the deviation is as measured in both axes perpendicular to the mean cellular anisotropic axis of the plurality of cells of the aOBB. In some embodiments of any of the aspect, the cells have a cellular anisotropic axis within a certain number standard deviations of a mean cellular anisotropic axis of the plurality of cells of the aOBB in one axis perpendicular to the mean cellular anisotropic axis of the plurality of cells of the aOBB (e.g., if the mean cellular anisotropic axis of the plurality of cells of the aOBB is the x axis, the deviation is in the y or the z axis). In some embodiments of any of the aspect, the cells have a cellular anisotropic axis within a certain number of standard deviations of a mean cellular anisotropic axis of the plurality of cells of the aOBB in the first axis perpendicular to the mean cellular anisotropic axis of the plurality of cells of the aOBB and within that same number of standard deviations of the mean cellular anisotropic axis of the plurality of cells of the aOBB in the second axis that is perpendicular to the mean cellular anisotropic axis of the plurality of cells of the aOBB (e.g., if the mean cellular anisotropic axis of the plurality of cells of the aOBB is the x axis, the deviation is in the y axis and also in the z axis).

In certain embodiments, the aOBBs may be composed of cellularly aligned, induced pluripotent stem cell-derived cardiomyocytes (iPSC-CMs).

In some embodiments, the cells are aligned in the aOBBs. By aligning the anisotropic aOBBs, the cells are also aligned parallel to the direction of the print path. The printing does not influence cells to change shape from spherical to oriented, it just aligns the aOBBs.

In some embodiments of any of the aspects, the bioink material comprises 10-1000e6 cells/ml. In some embodiments of any of the aspects, the bioink material comprises 10-500e6 cells/ml. In some embodiments of any of the aspects, the bioink material comprises at least 10e6 cells/ml. In some embodiments of any of the aspects, the bioink material comprises at least 100e6 cells/ml. In some embodiments of any of the aspects, the bioink material comprises at least 500e6 cells/ml.

In some embodiments of any of the aspects, wherein the plurality of aOBBs are at least 1% volume of the bioink material. In some embodiments of any of the aspects, wherein the plurality of aOBBs are at least 5% volume of the bioink material. In some embodiments of any of the aspects, wherein the plurality of aOBBs are at least 10% volume of the bioink material.In some embodiments of any of the aspects, wherein the plurality of aOBBs are at least 20% volume of the bioink material. In some embodiments of any of the aspects, wherein the plurality of aOBBs are at least 25% volume of the bioink material.

In some embodiments of any of the aspects, wherein the plurality of aOBBs are no more than 75% volume of the bioink material. In some embodiments of any of the aspects, wherein the plurality of aOBBs are no more than 70% volume of the bioink material. In some embodiments of any of the aspects, wherein the plurality of aOBBs are no more than 65% volume of the bioink material.

In some embodiments of any of the aspects, wherein a cellularly dense bioink material comprises a plurality of aOBBs which are at least 1% volume of the bioink material. In some embodiments of any of the aspects, wherein a cellularly dense bioink material comprises a plurality of aOBBs which are at least 5% volume of the bioink material. In some embodiments of any of the aspects, wherein a cellularly dense bioink material comprises a plurality of aOBBs which are at least 10% volume of the bioink material. In some embodiments of any of the aspects, wherein a cellularly dense bioink material comprises a plurality of aOBBs which are at least 20% volume of the bioink material. In some embodiments of any of the aspects, wherein a cellularly dense bioink material comprises a plurality of aOBBs which are at least 25% volume of the bioink material.

As used herein, “bioink” refers to a material comprising a plurality of aOBBs and at least one carrier fluid. The fluidity, rheology, and density of a bioink may vary with temperature, shear forces, compaction, or polymerization as described herein.

In the methods described herein, the plurality of aOBBs of a bioink material are aligned when the bioink material has been applied to a support. The plurality of aOBBs can be aligned prior to application or during the application process. As described herein, the inventors have found that shear forces will cause alignment of the aOBBs in a bioink. The aOBBs in a bioink can therefore be aligned by flowing the bioink material.

As used herein to refer to a plurality of aOBBs, “aligned” refers to the plurality of aOBBs displaying some degree of alignment of their individual anisotropic axes, e.g., a degree of alignment which is statistically not random. Methods of preparing such aligned aOBBs are provided elsewhere herein.

In some embodiments of any of the aspects, at least 90% of the plurality of aligned aOBBs have an aOBB anisotropic axis that is within +/−60 degrees of a mean aOBB anisotropic axis of the plurality of aligned aOBBs. In some embodiments of any of the aspects, at least 90% of the plurality of aligned aOBBs have an aOBB anisotropic axis that is within +/−50 degrees of a mean aOBB anisotropic axis of the plurality of aligned aOBBs. In some embodiments of any of the aspects, at least 90% of the plurality of aligned aOBBs have an aOBB anisotropic axis that is within +/−40 degrees of a mean aOBB anisotropic axis of the plurality of aligned aOBBs. In some embodiments of any of the aspects, at least 90% of the plurality of aligned aOBBs have an aOBB anisotropic axis that is within +/−30 degrees of a mean aOBB anisotropic axis of the plurality of aligned aOBBs. In some embodiments of any of the aspects, at least 90% of the plurality of aligned aOBBs have an aOBB anisotropic axis that is within +/−20 degrees of a mean aOBB anisotropic axis of the plurality of aligned aOBBs. In some embodiments of any of the aspects, at least 90% of the plurality of aligned aOBBs have an aOBB anisotropic axis that is within +/−10 degrees of a mean aOBB anisotropic axis of the plurality of aligned aOBBs.

In some embodiments of any of the aspects, at least 60% of the plurality of aligned aOBBs have an aOBB anisotropic axis that is within +/−30 degrees of a mean aOBB anisotropic axis of the plurality of aligned aOBBs. In some embodiments of any of the aspects, at least 60% of the plurality of aligned aOBBs have an aOBB anisotropic axis that is within +/−20 degrees of a mean aOBB anisotropic axis of the plurality of aligned aOBBs. In some embodiments of any of the aspects, at least 60% of the plurality of aligned aOBBs have an aOBB anisotropic axis that is within +/−10 degrees of a mean aOBB anisotropic axis of the plurality of aligned aOBBs.

In some embodiments of any of the aspect, the degrees are as measured in one axis perpendicular to the mean anisotropic axis. In some embodiments of any of the aspect, the degrees are as measured in both axes perpendicular to the mean anisotropic axis. In some embodiments of any of the aspect, the aOBBs have an aOBB anisotropic axis within a certain number of degrees of a mean aOBB anisotropic axis in one axis perpendicular to the mean aOBB anisotropic axis (e.g., if the mean aOBB anistropic axis is the x axis, the degrees are in the y or the z axis). In some embodiments of any of the aspect, the aOBBs have an aOBB anisotropic axis within a certain number of degrees of a mean aOBB anisotropic axis in the first axis perpendicular to the mean aOBB anisotropic axis and within that same number of degrees of the mean aOBB anisotropic axis in the second axis that is perpendicular to the mean aOBB anisotropic axis (e.g., if the mean aOBB anistropic axis is the x axis, the degrees are in the y axis and also in the z axis).

In some embodiments of any of the aspects, at least 50% of the plurality of aligned aOBBs have an aOBB anisotropic axis that is within 2 standard deviations of a mean aOBB anisotropic axis of the plurality of aligned aOBBs. In some embodiments of any of the aspects, at least 60% of the plurality of aligned aOBBs have an aOBB anisotropic axis that is within 2 standard deviations of a mean aOBB anisotropic axis of the plurality of aligned aOBBs. In some embodiments of any of the aspects, at least 70% of the plurality of aligned aOBBs have an aOBB anisotropic axis that is within 2 standard deviations of a mean aoBB anisotropic axis of the plurality of aligned aOBBs. In some embodiments of any of the aspects, at least 80% of the plurality of aligned aOBBs have an aOBB anisotropic axis that is within 2 standard deviations of a mean aOBB anisotropic axis of the plurality of aligned aOBBs. In some embodiments of any of the aspects, at least 90% of the plurality of aligned aOBBs have an aOBB anisotropic axis that is within 2 standard deviations of a mean aOBB anisotropic axis of the plurality of aligned aOBBs. In some embodiments of any of the aspects, at least 95% of the plurality of aligned aOBBs have an aOBB anisotropic axis that is within 2 standard deviations of a mean aOBB anisotropic axis of the plurality of aligned aOBBs.

In some embodiments of any of the aspects, at least 50% of the plurality of aligned aOBBs have an aOBB anisotropic axis that is within 1 standard deviation of a mean aOBB anisotropic axis of the plurality of aligned aOBBs. In some embodiments of any of the aspects, at least 60% of the plurality of aligned aOBBs have an aOBB anisotropic axis that is within 1 standard deviation of a mean aOBB anisotropic axis of the plurality of aligned aOBBs. In some embodiments of any of the aspects, at least 70% of the plurality of aligned aOBBs have an aOBB anisotropic axis that is within 1 standard deviation of a mean aoBB anisotropic axis of the plurality of aligned aOBBs. In some embodiments of any of the aspects, at least 80% of the plurality of aligned aOBBs have an aOBB anisotropic axis that is within 1 standard deviation of a mean aOBB anisotropic axis of the plurality of aligned aOBBs. In some embodiments of any of the aspects, at least 90% of the plurality of aligned aOBBs have an aOBB anisotropic axis that is within 1 standard deviation of a mean aOBB anisotropic axis of the plurality of aligned aOBBs.

In some embodiments of any of the aspect, the deviation is as measured in one axis perpendicular to the mean anisotropic axis. In some embodiments of any of the aspect, the deviation is as measured in both axes perpendicular to the mean anisotropic axis. In some embodiments of any of the aspect, the aOBBs have an aOBB anisotropic axis within a certain number standard deviations of a mean aOBB anisotropic axis in one axis perpendicular to the mean aOBB anisotropic axis (e.g., if the mean aOBB anistropic axis is the x axis, the deviation is in the y or the z axis). In some embodiments of any of the aspect, the aOBBs have an aOBB anisotropic axis within a certain number of standard deviations of a mean aOBB anisotropic axis in the first axis perpendicular to the mean aOBB anisotropic axis and within that same number of standard deviations of the mean aOBB anisotropic axis in the second axis that is perpendicular to the mean aOBB anisotropic axis (e.g., if the mean aOBB anistropic axis is the x axis, the deviation is in the y axis and also in the z axis).

The alignment of the aOBBs can also be considered with respect to the flow a bioink material is subjected to or the structures formed by the bioink material when applied to a support.

In some embodiments of any of the aspects, at least 90% of the plurality of aligned aOBBs have an aOBB anisotropic axis that is within +/−70 degrees of an anisotropic axis of the applied bioink material. In some embodiments of any of the aspects, at least 90% of the plurality of aligned aOBBs have an aOBB anisotropic axis that is within +/−60 degrees of an anisotropic axis of the applied bioink material. In some embodiments of any of the aspects, at least 90% of the plurality of aligned aOBBs have an aOBB anisotropic axis that is within +/−50 degrees of an anisotropic axis of the applied bioink material. In some embodiments of any of the aspects, at least 90% of the plurality of aligned aOBBs have an aOBB anisotropic axis that is within +/−40 degrees of an anisotropic axis of the applied bioink material. In some embodiments of any of the aspects, at least 90% of the plurality of aligned aOBBs have an aOBB anisotropic axis that is within +/−30 degrees of an anisotropic axis of the applied bioink material. In some embodiments of any of the aspects, at least 90% of the plurality of aligned aOBBs have an aOBB anisotropic axis that is within +/−20 degrees of an anisotropic axis of the applied bioink material. In some embodiments of any of the aspects, at least 90% of the plurality of aligned aOBBs have an aOBB anisotropic axis that is within +/−10 degrees of an anisotropic axis of the applied bioink material.

In some embodiments of any of the aspects, at least 60% of the plurality of aligned aOBBs have an aOBB anisotropic axis that is within +/−40 degrees of an anisotropic axis of the applied bioink material. In some embodiments of any of the aspects, at least 60% of the plurality of aligned aOBBs have an aOBB anisotropic axis that is within +/−30 degrees of an anisotropic axis of the applied bioink material. In some embodiments of any of the aspects, at least 60% of the plurality of aligned aOBBs have an aOBB anisotropic axis that is within +/−20 degrees of an anisotropic axis of the applied bioink material. In some embodiments of any of the aspects, at least 60% of the plurality of aligned aOBBs have an aOBB anisotropic axis that is within +/−10 degrees of an anisotropic axis of the applied bioink material.

In some embodiments of any of the aspects, the anisotropic axis of the applied bioink material is formed along the direction of flow the bioink material is subjected to.

In some embodiments of any of the aspects, the degrees are as measured in one axis perpendicular to the anisotropic axis of the applied bioink material. In some embodiments of any of the aspects, the degrees are as measured in both axes perpendicular to the anisotropic axis of the applied bioink material. In some embodiments of any of the aspects, the aOBBs have an aOBB anisotropic axis within a certain number of degrees of anisotropic axis of the applied bioink material in one axis perpendicular to the anisotropic axis of the applied bioink material (e.g., is the anisotropic axis of the applied bioink material is the x axis, the degrees are in the y or the z axis). In some embodiments of any of the aspects, the aOBBs have an aOBB anisotropic axis within a certain number of degrees of a anisotropic axis of the applied bioink material in the first axis perpendicular to the anisotropic axis of the applied bioink material and within that same number of degrees of the anisotropic axis of the applied bioink material in the second axis that is perpendicular to the anisotropic axis of the applied bioink material (e.g., is the anisotropic axis of the applied bioink material is the x axis, the degrees are in the y axis and also in the z axis).

As used herein, “carrier fluid” refers to a biocompatible fluid. Exemplary carrier fluids include water, cellular media, saline solutions, and the like. In some embodiments, the carrier fluid can further comprise polymers, binding polypeptides, and the like. In some embodiments of any of the aspects, the carrier fluid can comprise one or more of gelatin, collagen, fibrinogen, alginate, RGD modified alginate, or mixtures of any of the foregoing.

The term “cell culture medium” (also referred to herein as a “culture medium” or “medium” or “media”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. The appropriate cell culture media, for a particular cell type, is known to those skilled in the art. By way of non-limiting example, suitable cell culture media are available commercially from Millipore Sigma (St. Louis, MO) (Cat. No. C-22070) and 3H Biomedical (Uppsala. Sweden) (Cat. No. SC6201-2) and myocyte culture requirements are well known in the art (see, e.g., Louch et al. J Mol Cell Cardiol 201151:288-298); which is incorporated by reference herein in its entirety).

In some embodiments, the carrier fluid comprises one or more of gelatin, fibrinogen, collagen, alginate, RGD-modified alginate, fibrin, and cell media.

In some embodiments, the carrier fluid comprises one or more of processed gelatin, gelatin, fibrinogen, and cell media. In some embodiments, the carrier fluid comprises gelatin, fibrinogen, and cell media. In some embodiments, the carrier fluid comprises gelatin and fibrinogen. In some embodiments, the carrier fluid comprises gelatin and cell media. In some embodiments, the carrier fluid comprises fibrinogen and cell media. In some embodiments, the carrier fluid comprises gelatin. In some embodiments, the carrier fluid comprises fibrinogen. In some embodiments, the carrier fluid comprises cell media.

The methods described herein relate to applying a bioink material to a support. As used herein, “applying” refers to the act of causing the bioink material to come into contact with the support. Applying can include extruding, flowing, injecting, printing, and the like.

In some embodiments of any of the aspects, applying comprises extruding, flowing, or injecting the bioink material onto or into the support.

In some embodiments of any of the aspects, applying comprises extruding the bioink material onto or into the support. As used herein, “extrude” refers to a process of forcing a material through a constricted opening, outlet, or die.

In some embodiments of any of the aspects, applying comprises flowing the bioink material onto or into the support. As used herein, “flowing” refers to causing a fluid material to move in a desired direction. Flowing can relate to continuous movement of the fluid through a mechanism or flowing means. Flowing can relate to continuous movement of the fluid at a steady rate through a mechanism or flowing means. Flowing can be controlled by pressure, gravity, capillary forces, movement of a flowing mechanism or means, temperature and the like. Flowing can comprise extrusion.

When a fluid is subjected to flow, shear forces may be present. In some embodiments of any of the aspects, flowing a bioink material comprises exposing the bioink material to shear forces. In some embodiments of any of the aspects, flowing a bioink material comprises exposing the bioink material to an aligning flow. The strength and presence of the shear forces will be dependent on the density and viscosity of the fluid as well as the rate of flow, the size and arrangement of the flowing mechanism, as well as the temperature (both ambient and of the fluid and flowing mechanism). As described herein, application of shear forces to a bioink can cause the aOBBs to align within the bioink. The necessary shear forces will vary depending on the size and concentration of the aOBBs, the density and viscosity of the bioink the rate of flow, the size and arrangement of the flowing mechanism, as well as the temperature (both ambient and of the bioink and flowing mechanism). Methods for detecting and quantifying the alignment of flowed aOBBs are provided and demonstrated herein and one of skill in the art can modulate the foregoing flowing parameters to provide an “aligning” flow that will provide a desired degree of alignment.

In some embodiments of any of the aspects, the methods described herein can comprise flowing the bioink material prior to the applying step. In some embodiments of any of the aspects, the methods described herein can comprise flowing the bioink material concurrently with the applying step.

In some embodiments of any of the aspects, applying the bioink material comprises moving the bioink material through a nozzle, die, channel, or tube. In some embodiments of any of the aspects, applying the bioink material comprises moving the bioink material through a nozzle. As used herein, the term “channel” refers to any capillary, channel, tube, or groove that is deposed within or upon a substrate. A channel can be a microchannel; i.e. a channel that is sized for passing through microvolumes of liquid. As used herein, the term “nozzle” refers to an outlet in a channel, tube, or reservoir which is constricted as compared to the cross-section of the channel, tube, or reservoir.

In some embodiments of any of the aspects, bioink material is applied in the form of one or more filaments. As used herein, “filament” refers to a structure in which the overall length of the structure is more than 10× the width or height of the structure at any point along the length. Multiple filaments can be applied to a support in contact or proximity to each other, forming superstructures. For example, a plurality of filaments applied in parallel in a single layer can form a superstructure sheet. As another example, a plurality of filaments applied vertically can form a cuboidal superstructure.

In some embodiments of any of the aspects, the one or more filaments have a cross-sectional diameter no more than 20× the diameter of an application terminus of the nozzle, die, channel, or tube (e.g., the diameter of the point or outlet at which the bioink material leaves the nozzle, die, channel, or tube). In some embodiments of any of the aspects, the one or more filaments have a cross-sectional diameter no more than 10× the diameter of an application terminus of the nozzle, die, channel, or tube (e.g., the diameter of the point or outlet at which the bioink material leaves the nozzle, die, channel, or tube).In some embodiments of any of the aspects, the one or more filaments have a cross-sectional diameter no more than 200% the diameter of an application terminus of the nozzle, die, channel, or tube (e.g., the diameter of the point or outlet at which the bioink material leaves the nozzle, die, channel, or tube). In some embodiments of any of the aspects, the one or more filaments have a cross-sectional diameter no more than 150% the diameter of an application terminus of the nozzle, die, channel, or tube (e.g., the diameter of the point or outlet at which the bioink material leaves the nozzle, die, channel, or tube). In some embodiments of any of the aspects, the one or more filaments have a cross-sectional diameter no more than 110% the diameter of an application terminus of the nozzle, die, channel, or tube (e.g., the diameter of the point or outlet at which the bioink material leaves the nozzle, die, channel, or tube). In some embodiments of any of the aspects, the one or more filaments have a cross-sectional diameter which is 0.2 to 5× the inner diameter of an application terminus of the nozzle, die, channel, or tube (e.g., the diameter of the point or outlet at which the bioink material leaves the nozzle, die, channel, or tube). In some embodiments of any of the aspects, the one or more filaments have a cross-sectional diameter which is 1 to 3× the inner diameter of an application terminus of the nozzle, die, channel, or tube (e.g., the diameter of the point or outlet at which the bioink material leaves the nozzle, die, channel, or tube)

In some embodiments of any of the aspects, bioink material is applied in the form of one or more filaments by moving the bioink material through a nozzle. In some embodiments of any of the aspects, the one or more filaments have a cross-sectional diameter no more than 200% the diameter of an application terminus of the nozzle. In some embodiments of any of the aspects, the one or more filaments have a cross-sectional diameter no more than 1500% the diameter of an application terminus of the nozzle. In some embodiments of any of the aspects, the one or more filaments have a cross-sectional diameter no more than 110% the diameter of an application terminus of the nozzle. In some embodiments of any of the aspects, the one or more filaments have a cross-sectional diameter which is 0.2 to 5× the inner diameter of an application terminus of the nozzle. In some embodiments of any of the aspects, the one or more filaments have a cross-sectional diameter which is 1 to 3× the inner diameter of an application terminus of the nozzle.

In some embodiments, due to the same forces that aligned a-cellular filaments printed by Kim et al., which is incorporated herein by reference in its entirety, aOBBs aligned parallel to the print path when extruded through a tapered nozzle. Using this method, complex tissue structures were generated that aligned at the aOBB, cellular, and sarcomeric length scales. Such embodiments are described in more detail below herein.

As used herein, “support” refers to a material having a biocompatible surface. In some embodiments of any of the aspects, the support can comprise a particle (including, but not limited to an agarose or latex bead or particle or a magnetic particle), a bead, a nanoparticle, a polymer, a substrate, a slide, a coverslip, a plate, a dish, a well, a membrane, and/or a grating. The support can include many different materials including, but not limited to, polymers, plastics, resins, polysaccharides, silicon or silica based materials, carbon, metals, inorganic glasses, and membranes. In some embodiments of any of the aspects, the support is a glass support. In some embodiments of any of the aspects, the support is a plastic support. In some embodiments of any of the aspects, the support is a biological hydrogel or matrix. In some embodiments of any of the aspects, the support is a hydrogel or matrix. The use of “support” does not imply a particular singular physical arrangement of the support and the bioink. For example, bioink can be printed bottom up onto the top of a support, but bioink can also be flowed into a support which is in the form of an enclosure or mold.

In some embodiments of any of the aspects, the support does not comprise an anisotropic scaffold. In some embodiments of any of the aspects, the support does not comprise an scaffold with anisotropic channels, voids, or recesses. In some embodiments of any of the aspects, the support does not comprise a surface with anisotropic channels, voids, or recesses.

The underlying printing substrate, e.g., support, may be any suitable known substrate, such as plastic (e.g., acrylic), quartz, or glass, or any biological hydrogel. The underlying substrate may be plasma-treated or coated with a layer of at least one of Matrigel®, poly L-lysine, Geltrex™, gelatin, nitrogen, fibronectin, collagen 1, collagen IV, fibrinogen, gelatin methacrylate, fibrin, silk, pegylated gels, collagen methacrylate, basement membrane proteins, or any other biomaterial. The substrate may be any combination of gelatin, fibrin, or collagen I, or any other basement membrane proteins.

In certain embodiments, the matrix may be a granular matrix composed of synthetic (pluronic) or biological polymer, as well as a living matrix composed of building blocks.

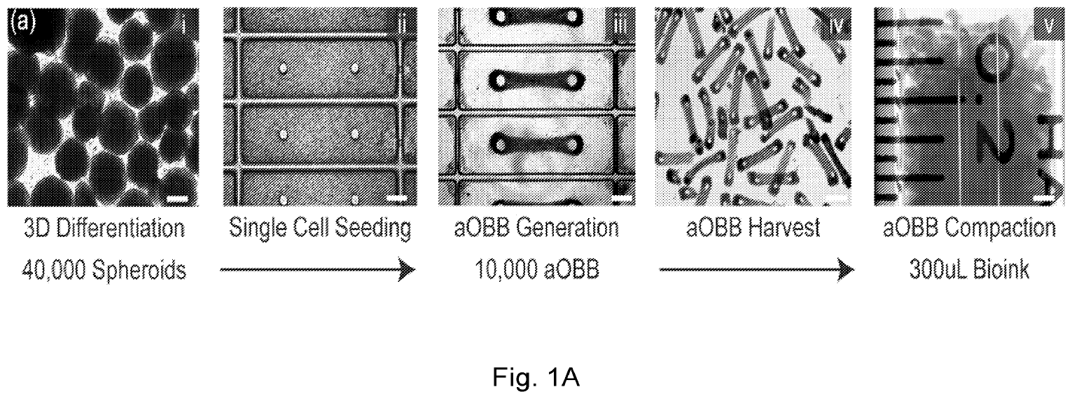

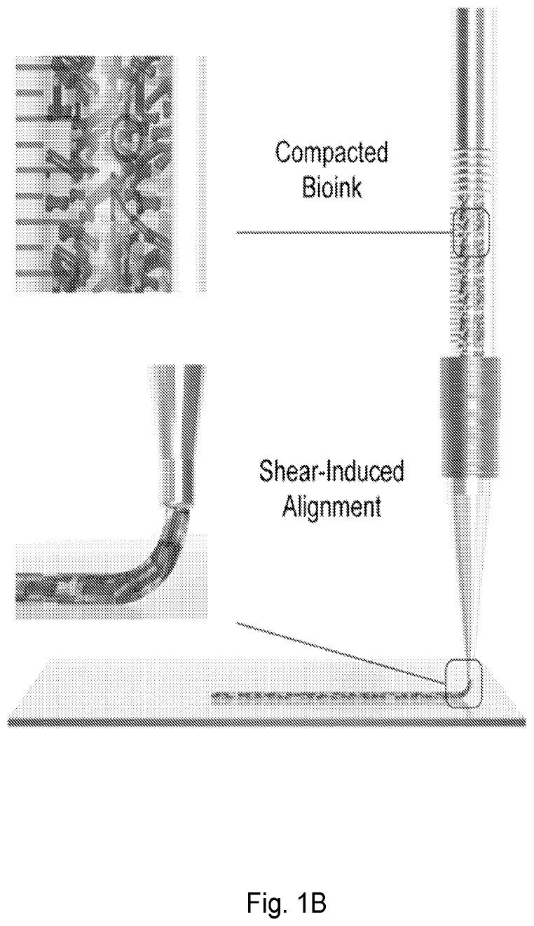

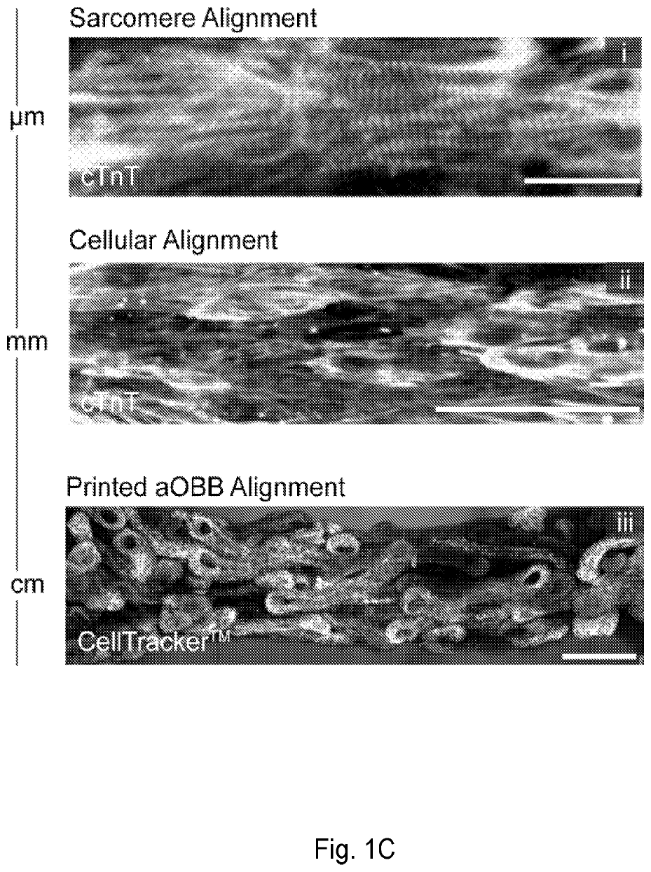

In exemplary embodiments, described herein is the fabrication of engineered cardiac tissue with programmable alignment via bioprinting of anisotropic organ building blocks (aOBBs) (FIG. 1). In some embodiments, the anisotropic organ building block (aOBB) can refer to a rod-like tissue including extracellular matrix and cells aligned parallel to the long axis. In such embodiments, these aOBBs are elongated micro-tissues composed of cellularly aligned, human induced pluripotent stem cell-derived cardiomyocytes (hiPSC-CMs) that can be modularly assembled into a printable bioink. Individual aOBBs within this bioink align along the print path due to the same shear and extensional forces that orient acellular fibers upon extrusion through a tapered nozzle. Using this method, cardiac tissues with high cellular density and programmed alignment across multiple length scales may be fabricated; ranging from individual aOBBs to the sarcomeric machinery that drives their contractile function.

Within this three-dimensional context, more complex tissues could be constructed that mimic physiological structures. For example, within cardiovascular context, in certain embodiments, the method described herein can produce tissues that resemble the left ventricular helical alignment. This could be a patch or a chambered structure where alignment changes from the inside/interior (e.g., endocardial-like inside chamber) to the outside/exterior (e.g., epicardial-like exterior chamber). In certain other embodiments, radial aligned tissue could be generated using the described method, to mimic the radial alignment in the atria.

In some embodiments of any of the aspects, the methods described herein further comprise polymerizing the bioink material. In some embodiments of any of the aspects, the methods described herein further comprise polymerizing the bioink material after the applying step. In some embodiments of any of the aspects, the methods described herein further comprise polymerizing a bioink material that has been applied to or is in contact with a support.

As used herein, “polymerize” refers to the process of making a polymeric material that can be a homopolymer, copolymer, terpolymer, or the like and includes chain formation, chain growth, network formation, network growth, and polymer modification. Methods of polymerization are known in the art and include step-growth polymerization, chain polymerization, oxidation, cross-linking, and end-capping. Methods of polymerization ben be light-based, enzyme-based, temperature-based, etc. One of skill in the art can readily select an appropriate polymerization protocol on the basis of the polymer and type of polymerization desired.

In some embodiments of the described method, after printing, the tissue may be submerged in 4° C. chilled media loaded with thrombin. Upon incubation at elevated temperatures (37° C.), the thermally reversible gelatin melts, while simultaneously the thrombin diffuses into the tissue and polymerizes the fibrinogen into fibrin.

In some embodiments, the method includes extruding a bioink material through a nozzle onto a support to form a structure of the bioink material, the bioink material comprising a carrier fluid and anisotropic organ building blocks (aOBBs), the aOBBs comprising extracellular matrix material (ECM) and cellularly aligned cells, wherein at least some of the aOBBs align along a direction of extrusion in the structure of the bioink material; and polymerizing the structure of the bioink material, thereby forming the tissue or tissue construct having arbitrarily programmed alignment.

In some embodiments of any of the aspects, the methods described herein further comprise compacting the bioink material. In some embodiments of any of the aspects, the methods described herein further comprise compacting the bioink material before the applying step. In some embodiments of any of the aspects, the methods described herein further comprise compacting the bioink material before is has been applied to or is contact with a support. In some embodiments of any of the aspects, the methods described herein further comprise compacting the bioink material before it is subjected to flow. In some embodiments of any of the aspects, the methods described herein further comprise compacting the bioink material before it is subjected to an aligning flow.

As used herein, “compacting” refers to a process of increasing aOBB density or concentration within at least a portion of a solution or bioink material. Methods of compacting are known to one of ordinary skill in the art and include, without limitation, centrifugation, capillary flow to increase the aOBB density in the bioink material, or evaporation. In some embodiments of any of the aspects, compacting comprises centrifugation of the bioink material. Exemplary, but non-limiting centrifugation parameters include centrifugation for 30 seconds to 5 minutes at 10 to 300 g.

In some embodiments of any of the aspects, the methods described herein can comprise a first step (e.g., prior to applying the bioink material), of providing the bioink material comprising:
culturing the aOBBs;
contacting the aOBBs with the carrier fluid; and
compacting the aOBBs.

aOBBs can be prepared by any method of providing a combination of cells and polymer known in the art, e.g., by culturing cells in the presence of a polymer, or admixing cells and polymers.

In some embodiments, particularly those relating to aOBBs comprising cellularly aligned cells, the aOBBs can be prepared utilizing the pillar culture methods described herein. For example, in some embodiments of any of the aspects, a method described herein can further comprising a first step of providing the bioink material, comprising: culturing the aOBBs within a pillar array; contacting the aOBBs with at least one ROCK inhibitor; harvesting the aOBBs from the pillar array by manual pipetting or by degradation of the pillar array; resuspending the aOBBs in the carrier fluid; and compacting the aOBBs. In some embodiments of any of the aspects, a method described herein can further comprise a first step of providing the bioink material, comprising: culturing the aOBBs within a pillar array; harvesting the aOBBs from the pillar array by manual pipetting or by degradation of the pillar array; resuspending the aOBBs in the carrier fluid; compacting the aOBBs; and adjusting rheology of the bioink material by modulating temperature.

As used herein, “pillar array” refers to a device comprising multiple pillars. The device is preferably made of a biocompatible material. Exemplary pillar arrays for the provision of aOBBs comprising cardiomyocites are described in detail below herein.

ROCK inhibition is demonstrated herein to relax aOBBs, e.g., for removal from pillar arrays and/or flowing. Rho-associated coiled Kinases or Rho kinases (ROCKs) are a family of serine/threonine kinases involved in the regulation of the cellular cytoskeleton. The structure and function of ROCK polypeptides are further described, e.g., in Gervaise Loirand “Rho Kinases in Health and Disease” Pharmacological Reviews (2015), 67 (4) 1074-1095; DOI: https://doi.org/10.1124/pr.115.010595, Mackay and Hall J Biol Chem 1998, 273, 20685; Aspenstrom Curr Opin Cell Biol 1999, 11, 95; Amano, et al. Exp Cell Res 2000, 261, 44, each of which is incorporated herein by reference in their entireties. Nucleic acid and amino acid sequences for ROCK1 and ROCK2 are known in the art and can include, e.g., the sequences associated with NCBI Gene IDs: 6093, 9475, 19877, 19878, or an ortholog thereof. Inhibitors of ROCK1 and/or ROCK 2 can include but are not limited to those described, e.g., in Feng Yet al. “Rho Kinase (ROCK) Inhibitors and Their Therapeutic Potential.” J Med Chem. 2016 Mar. 24; 59(6):2269-300. doi: 10.1021/acs.jmedchem.5b00683, and WO2002076977A2, each of which is incorporated herein by reference their entireties.

Exemplary ROCK inhibitors include but are not limited to AT-13148, BA-210, beta-alemene, belumosudil, choman 1, DJ4, fasudil, GSK-576371, GSK429286A. H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983. In some embodiments of any of the aspects, the ROCK inhibitor is Y-27632. As used herein, “Y-27632” refers to a compound having the following structure or a pharmaceutically acceptable salt thereof.

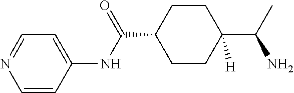 [SMILES: C[C@@H](N)[C@H]1CC[C@H](C(=O)Nc2ccncc2)CC1]

The synthesis of Y-27632 as well as the structure and synthesis of related compounds, e.g., derivatives, including those with similar biological activity are known in the art, see, e.g., WO2002076977A2; US-2004028716-AT; EP-2628482-A1; and Paleček J, et al. “A practical synthesis of Rho-Kinase Inhibitor Y-27632 and fluoro derivatives and their evaluation in human pluripotent stem cells.” Org Biomol Chem. 2011 Aug. 7; 9(15):5503-10. doi: 10.1039/clob05332a, the contents of each of which are incorporated herein by reference in their entireties.

To develop a printable volume of the bioink for use in the described method, a micropillar array platform may be manufactured to generate tens of thousands of aOBBs. Within these pillar arrays, the aOBBs could be cultured, characterized, and harvested on-demand.

The aOBBS may be cultured for at least 1 day and can be cultured indefinitely, and until the culturing is no longer desired. In some embodiments, aOBBS can be grown for 30 days or longer, e.g., for 2 months, 3 months, 6 months, 9 months, 12 months, 24 months, 30 months, 36 months, 42 months, etc. Any time periods in between the mentioned time periods for culturing the cells are also contemplated

In certain embodiments, the aOBBs may be cultured and characterized within the described pillar arrays.

Traditionally, micropillar arrays are fabricated using photolithography, which can be a laborious, technical and expensive process to iterate. In contrast, the inventors employed stereolithography (SLA) 3D printing to quickly prototype design criteria such as well dimensions, pillar roughness, and aOBB aspect ratio. To scale the final design, a series of polymer molding processes was used to manufacture a single contiguous elastomeric plate with n=1050 micropillar wells. This plate was designed to fit inside Nunc™ OmniTray™ 1-well plates that are compatible with conventional imaging equipment. To the best of the inventors' knowledge, this plate provides the largest replicate value (n) of any pillar array published in literature, with potential parallel applications for disease modeling and drug discovery. Within three days of seeding, the self-assembled aOBBs were synchronously contractile and the iPSC-CMs demonstrated aligned sarcomeres as visualized by immunofluorescent staining and Fourier analysis.

In certain embodiments, the aOBBs may be harvested from the pillars by manual pipetting.

In order to prevent aOBB compaction upon removal from pillars, the aOBBs may be pre-incubated in 50 uM rock inhibitor (Y-27632) for a period of time, such as, e.g., 1 hour.

In certain other embodiments, the aOBBs could also be harvested by on-demand degradation of pillar arrays. For example, if the pillar arrays are manufactured using thermally reversible or enzymatically degradable polymer, such as hyaluronic acid, gelatin, pluronic, alginate, agarose, or other polysaccharide, the on-demand degradation releases the aOBBs and enables their harvest for downstream bioink development.

Other known methods of the aOBBs removal may also be utilized in the described method.

In certain embodiments, upon aOBB harvest, the aOBBs may be resuspended in a carrier fluid containing ECM to produce the bioink. The carrier fluid may include, e.g., processed gelatin, fibrinogen and cell media. The processed gelatin acts as a rheological modifier, while the fibrinogen acts as long-term structural support upon downstream polymerization.

To obtain high cellular density (about 10-500e6 cell/mL) within the bioink, the aOBBs may be centrifuged (30-300 g) within the syringe.

Prior to printing, the bioink rheology is tuned, e.g., if carrier fluid ECM contains gelatin then the syringe temperature is lowered.

During the printing process, shear and extensional flow fields that develop during extrusion through a tapered nozzle, align the aOBBs parallel to the print path. In other words, the aOBBs anisotropically align along a direction of extrusion in the structure of the bioink material. The phrase “the aOBBs align along a direction of extrusion in the structure of the bioink material” means that within the 3D printed filaments, the long axis' of the aOBBs are parallel to the direction of print. Taking advantage of this alignment phenomena, cellular alignment in arbitrarily complex patterns can be demonstrated.

In certain embodiments, in a two-dimensional context, printing on a substrate can generate filaments and sheets.

The application of the bioink material to the support can be performed in an order or path such that the bioink material forms a pattern or combination of patterns on the support. Exemplary patterns include, but are not limited to, linear, chevron, and spiral. In some embodiments of any of the aspects, a combination of patterns can be formed.

Patterns include, but are not limited to, unidirectional linear, convergent, bipennate, radial and spiral. In certain other embodiments, within a three-dimensional context, aOBBS could be embed printed within a supportive matrix to generate free-form structures.

In some embodiments of any of the aspects, a method described herein can further comprise culturing the applied bioink material to form a tissue or tissue construct. In some embodiments of any of the aspects, a method described herein can further comprise culturing the applied bioink material in or on the support to form a tissue or tissue construct.

The terms “tissue” or “tissue construct” can be used interchangeably and refer to a two-dimensional or a three-dimensional tissue culture created or synthesized by culturing one or several types of cells, e.g., human pluripotent or multipotent stem cells on, e.g., a substrate that have undergone a degree of differentiation.

In the context of the tissue or tissue construct produced by the described method, the tissue construct may have any desired 2D or 3D shape. For example, the tissue construct may have a planar geometry constructed from a single layer or multiple layers of anisotropic organ building blocks (aOBBs). Such structures may have any desired height (thickness). Typically, the tissue construct has a height of about 100 cm or less, about 10 cm or less, about 1 cm or less, about 1 mm or less, about 500 microns or less, or about 100 microns or less, and typically at least about 10 microns, at least about 100 microns, at least about 200 microns, or at least about 1 mm, with applications ranging from tissue cultures and drug screening to skin constructs and corneal replacements.

Alternatively, the tissue construct may have an arbitrary or application-dependent 3D size and shape. The tissue construct may have a solid structure, a porous structure, and/or a hollow structure (e.g., tubular or nontubular) and may be fabricated to mimic the morphology and function of particular organ. For example, the tissue or tissue construct may have the size, shape, morphology and/or function of a heart, kidney, pancreas, liver, bladder, urethra, trachea, esophagus, skin or other bodily organ. The 3D size and shape may in some cases be determined by a mold.

Certain embodiments relate to a method of forming a tissue or tissue construct having arbitrarily programmed alignment. The phrase “a tissue or tissue construct having arbitrarily programmed alignment” refers to the omnidirectional control over three-dimensional cellular alignment as directed by the print path.

As used herein, the term “culturing” refers to maintaining cells described herein in conditions suitable for cell viability, proliferation, and/or differentiation. For example, the cells present in the aOBBs of a bioink material described herein can be maintained in a culture medium on the support they are applied to, whether it be on plates, a tissue culture dish, a flask, a hydrogel or a polymer.

The tissue or tissue construct can be vascularized. In certain embodiments, the described method can be paired with previously developed vascularization strategies to generate multi-layered tissue structures. For example, U.S. Pat. No. 10,117,968, and U.S. Pat. Pub. Nos. US 2018/0030409, US 2020/0248147, and US 2020/0164109, are incorporated herein in their entirety.

Shown herein is a functional effect of alignment that results in statistically higher overall force, force/CM, and stress (force normalized by cross-sectional area). Also, shown herein is a higher conduction velocity. The inventors saw high and roughly equal alignment across order of magnitude of print speeds (1-10 mm/s) so they could not get a cutoff where % of aOBBs aligned=functional difference.

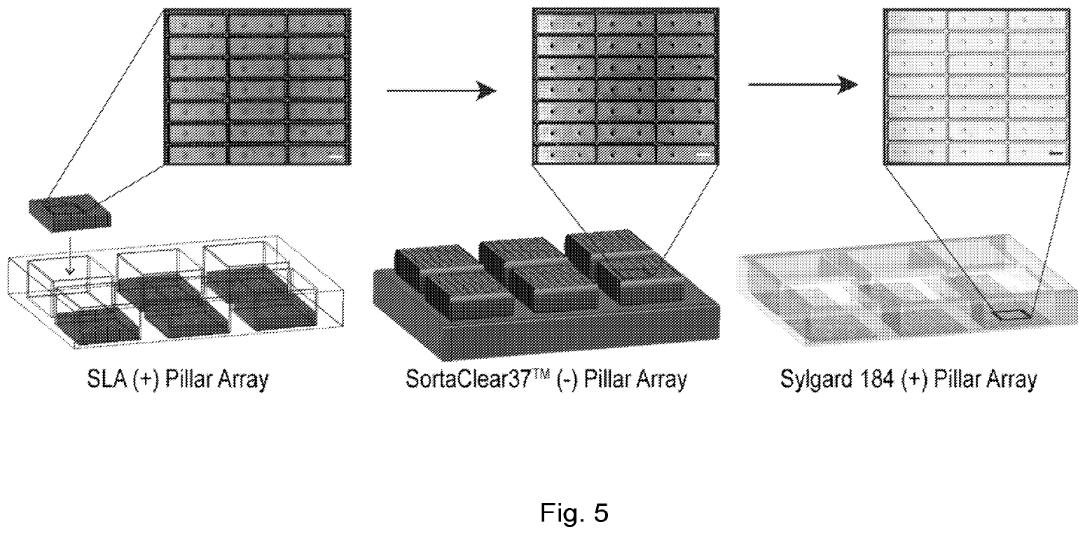

In certain embodiments, the first step in creating the described cardiac bioinks may be to fabricate scalable micro-pillar arrays by stereolithography (SLA). These micro-pillar arrays may be used to generate tens of thousands of aOBBs with controlled aspect ratio and cellular composition. After optimizing these parameters, a sequential transfer micro-molding process can be employed to create a single contiguous elastomeric plate with n=1050 micro-pillar wells (FIG. 5). To inventors' knowledge, this pillar platform provides the largest replicate value (n) reported to date.

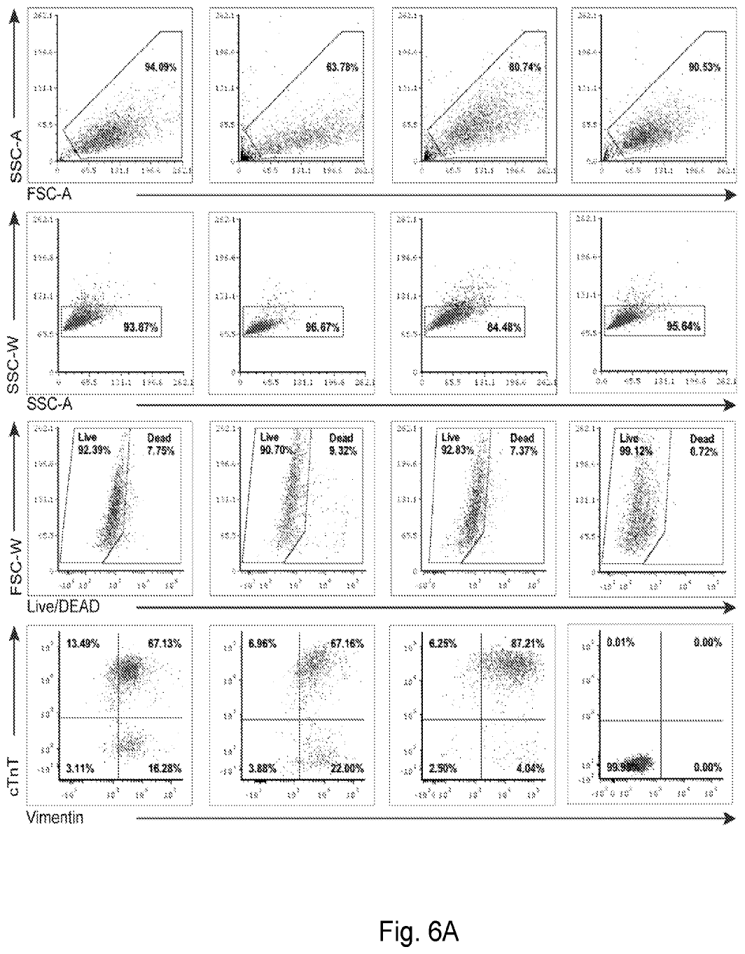

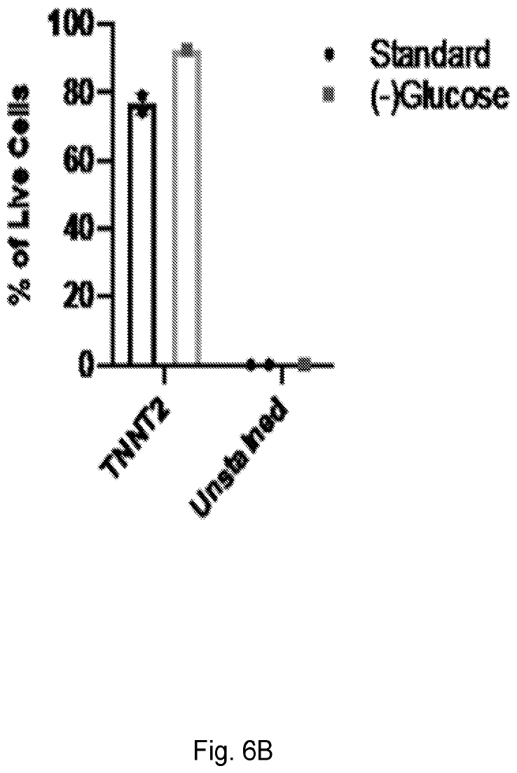

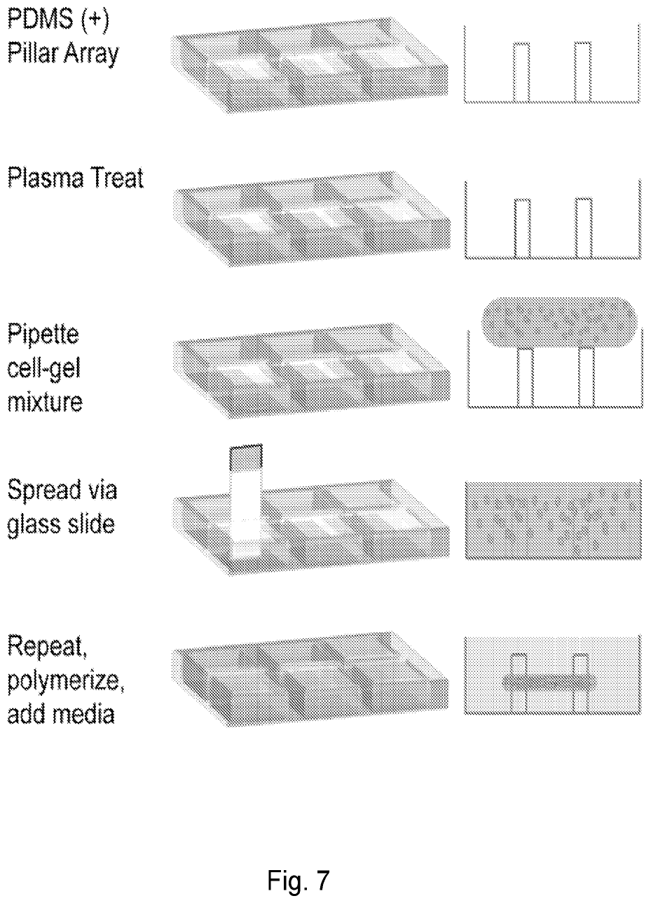

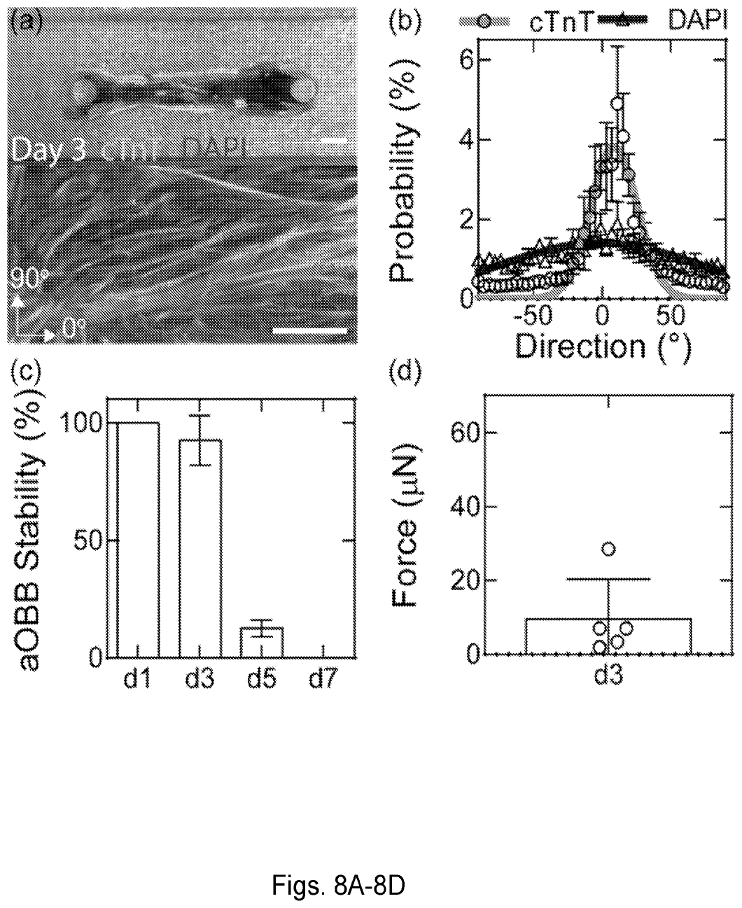

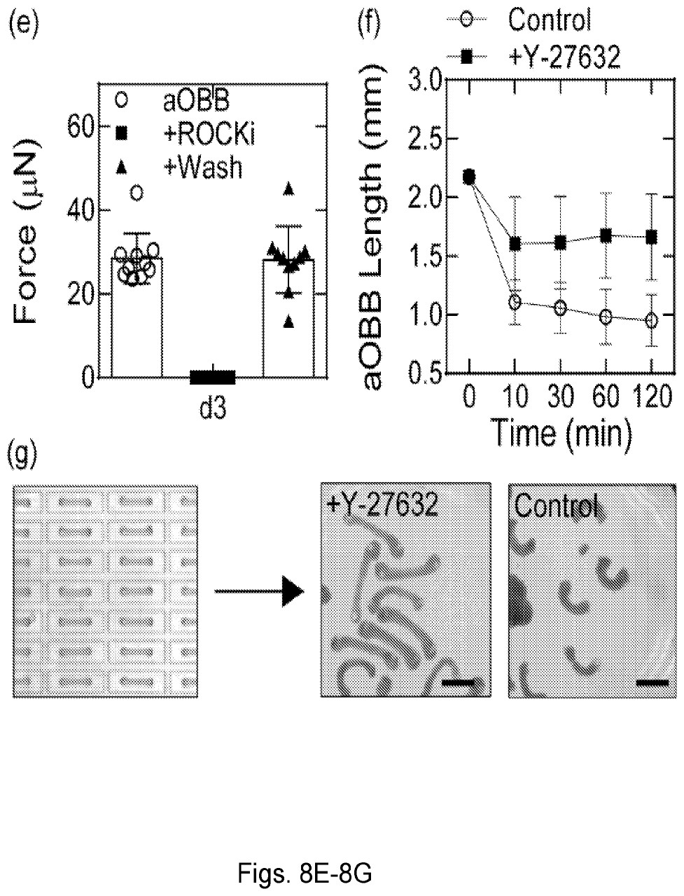

Next, to generate embryoid bodies from hiPSCs, which are subsequently differentiated into cardiac spheroids and dissociated into cardiomyocytes (FIGS. 6A-6B) a previously reported protocol can be followed. These hiPSC-CMs may then be combined with an appropriate number of human neonatal dermal fibroblasts (hNDFs) to achieve either a 0:1 (model) or 9:1 ratio. These cells may then be resuspended in a collagen I solution at a concentration of 10⁶ cells/mL and seeded into the micro-pillar arrays to generate cellularly dense, cardiac micro-tissues that serve as aOBBs (FIG. 7). Within three days of seeding (9:1 iPSC-CM:hNDF ratio), the self-assembled aOBBs are synchronously contractile and their iPSC-CMs exhibit aligned sarcomeres, as visualized by immunofluorescent staining, and quantified via Fourier analysis (FIGS. 8A-8G). After harvesting the aOBBs from the micro-pillar arrays, they may be resuspended in a mixture of processed gelatin and fibrinogen in which gelatin serves as a rheological modifier during printing, while fibrinogen provides structural support after polymerization. Finally, the aOBB-laden suspension may be compacted to create a cellularly-dense bioink.

To demonstrate the ability to direct aOBB alignment, macro-tissues may be bioprinted in both cylindrical and sheet-like geometries from bioinks composed of either fibroblast (0:1 iPSC-CM:hNDF ratio) or cardiac (9:1 iPSC-CM:hNDF ratio) aOBBs.

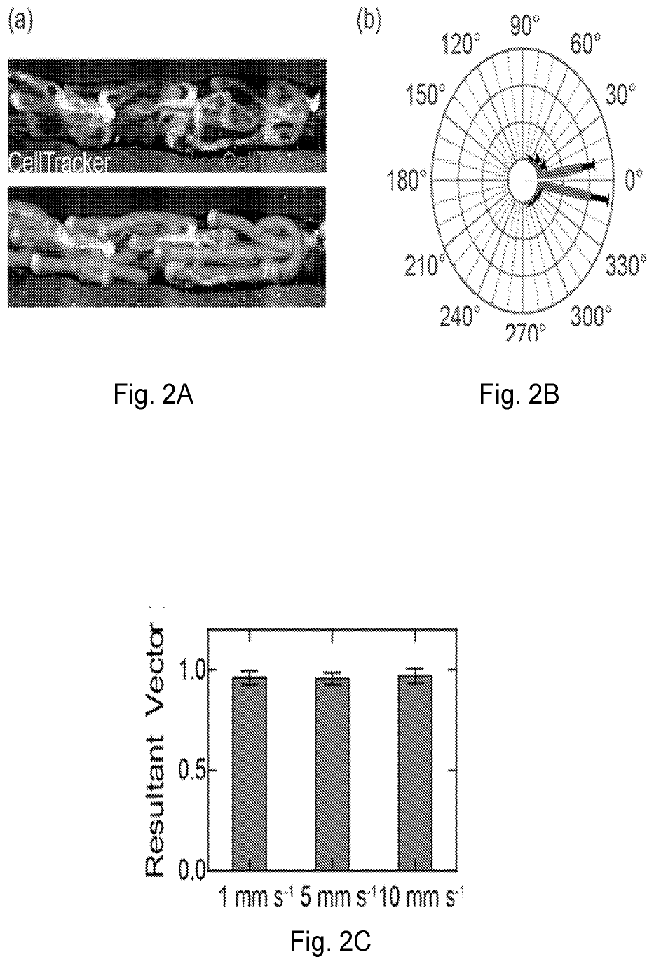

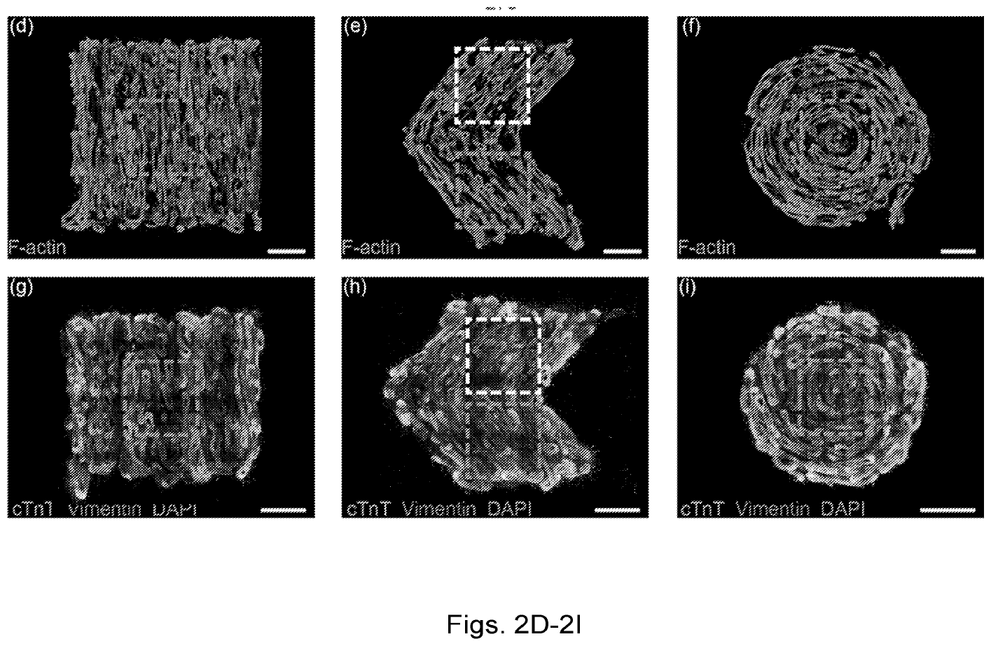

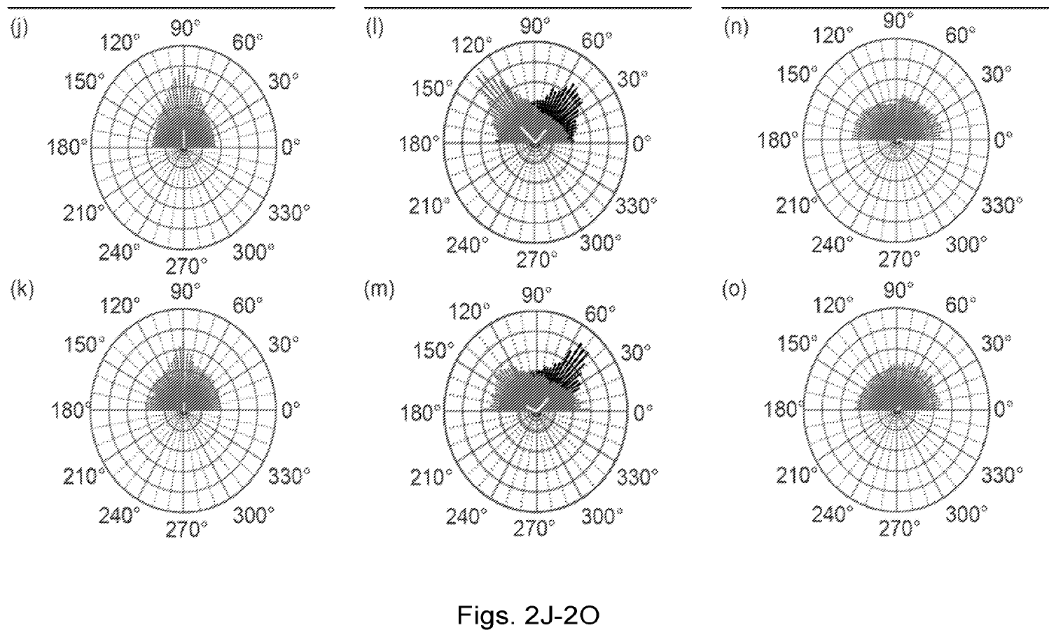

In certain embodiments, the aOBBs may be pre-labeled with fluorescent cell trackers and confocal microscopy may be used to visualize their alignment within the dense cylindrical tissues (FIGS. 2A-2O).

In certain embodiments, the alignment may be quantified by overlaying each aOBB with a volumetric 3D model and extracting their primary orientation relative to the print direction (FIG. 2A).

For example, for fibroblast-based cylindrical tissues bioprinted at 5 mm s⁻¹, it was found that the mean orientation angle of these aOBBs plotted in a polar histogram is centered around 0°, which corresponds to the printing direction (FIG. 2B). Importantly, a high degree of aOBB alignment across a broad range of print speeds (1-10 mm s⁻¹) was observed (FIG. 2C), indicating that these soft, cellularly dense aOBBs are capable of shear-induced alignment during bioprinting.

In certain embodiments, harnessing this capability, both fibroblast (FIGS. 2D-2F) and cardiac (FIGS. 2G-2I) macro-tissue sheets composed of linear, chevron, and spiral patterned architectures with programmed aOBB alignment may be created.

In certain embodiments, Fourier analysis may be applied to confocal images of immunofluorescently stained macro-tissue sheets to determine the primary orientation of aOBBs.

In certain embodiments, using polar histograms, their axial distribution, mean orientation angles, and resultant vector lengths may be characterized. The vertical patterned tissue sheets exhibited a mean orientation angle near 90° (FIGS. 2J, 2K), while the chevron patterned tissue sheets exhibited two primary orientation angles near 45°/135° (FIGS. 2L, 2M). Moreover, the distribution of aOBB orientations for the vertical and chevron patterned sheets demonstrated narrow peaks with correspondingly high resultant vector lengths, visualized as the length of the yellow bars overlaying the polar histograms. By contrast, the circular patterned sheets exhibited a much broader and more uniform distribution of orientation angles, as indicated by the associated low resultant vector lengths (FIGS. 2N, 20).

In certain embodiments, to facilitate long-term culture and functional characterization, cardiac macro-tissues composed of aOBBs may be printed onto custom-designed, elastomeric macro-pillar platforms (Movie S2). In addition, spheroidal OBBs may be printed that are assembled in pillarless arrays seeded with the same cellular and extracellular matrix composition as their anisotropic counterparts.

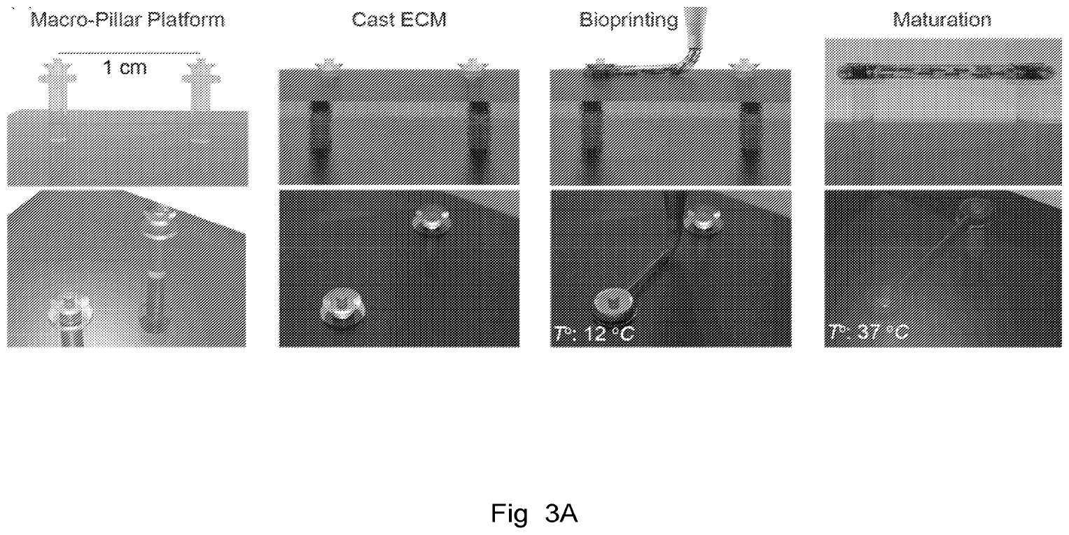

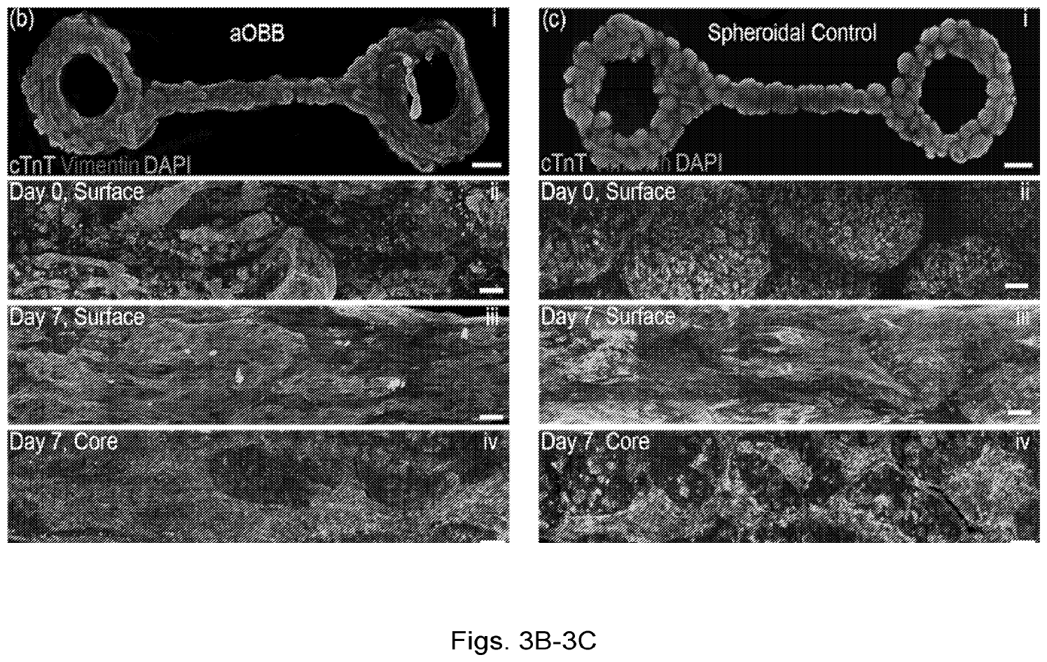

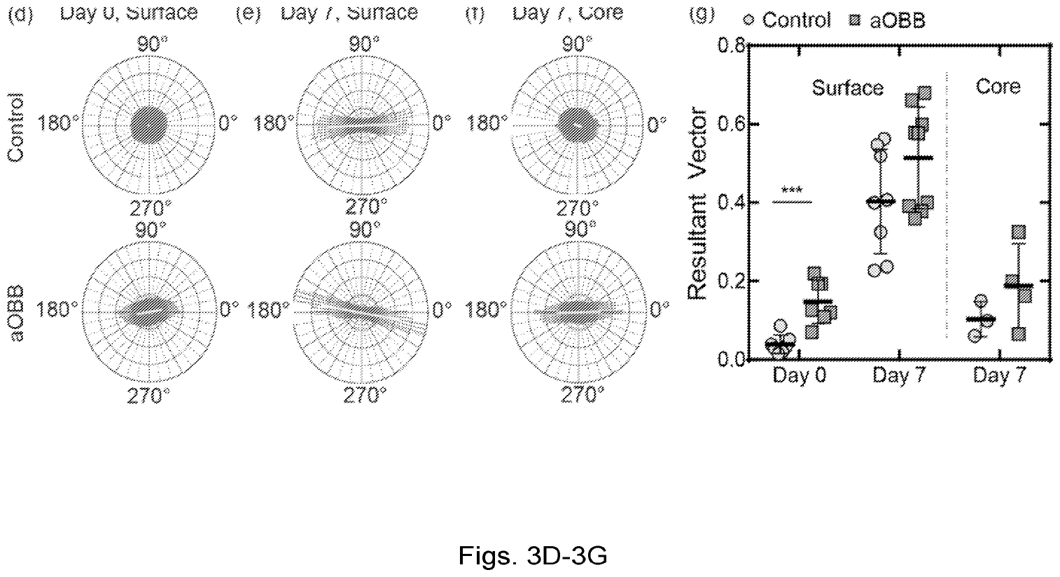

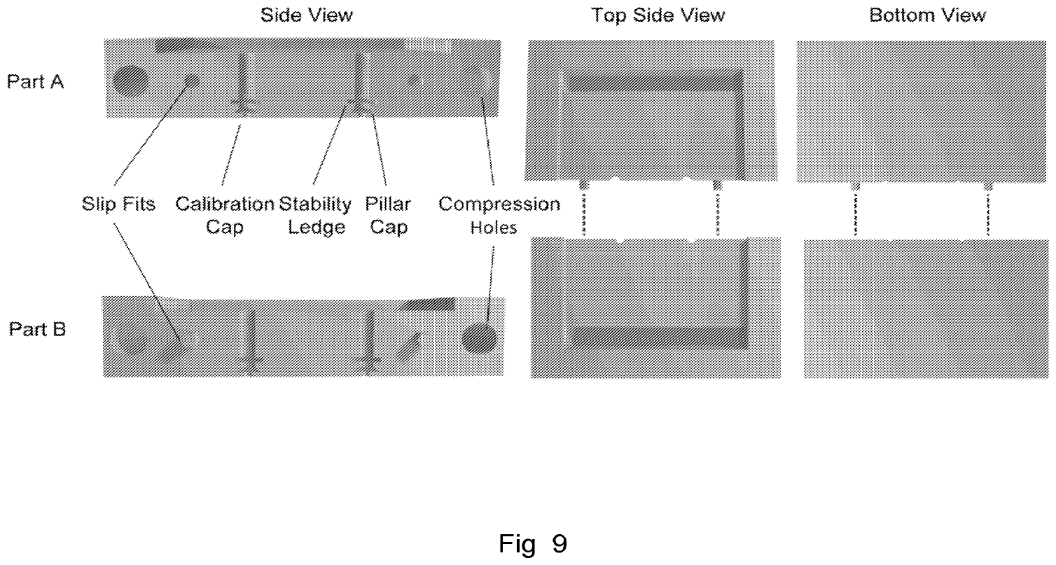

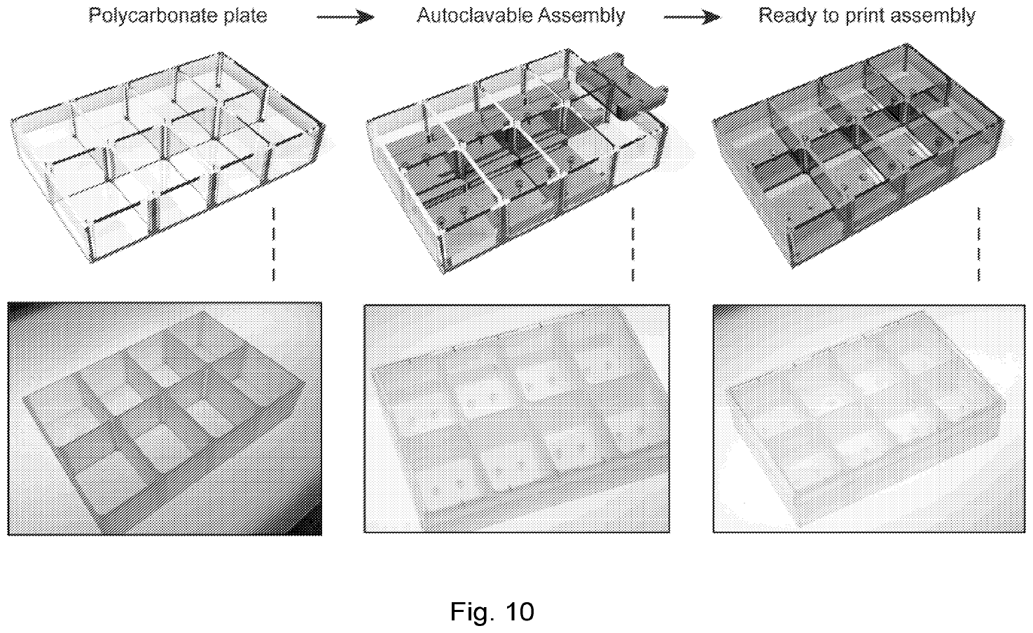

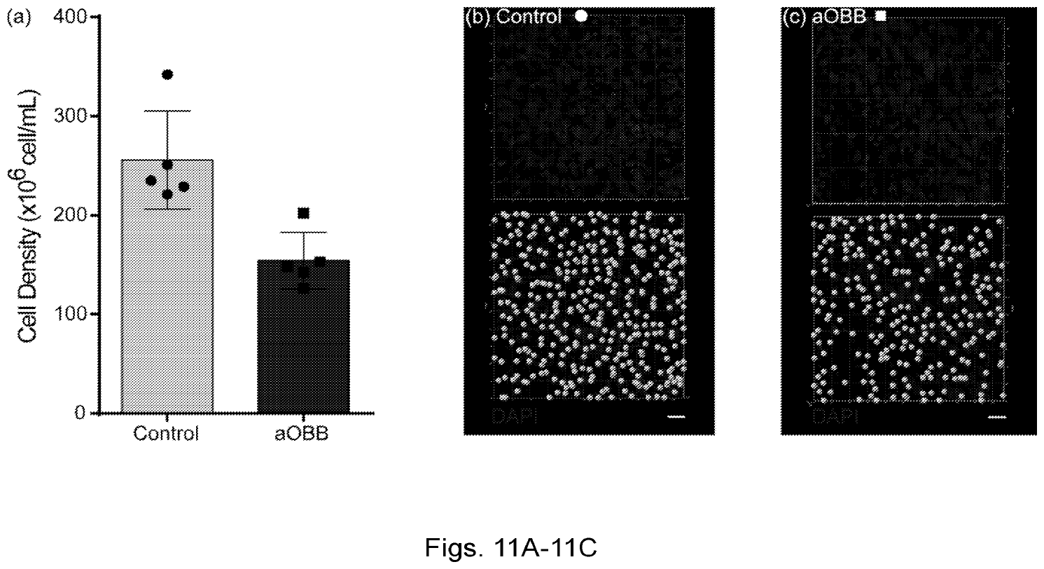

Upon compaction, the spheroids demonstrated isotropic cellular alignment and served as a control for downstream functional assays. The platforms are designed such that bioprinted cardiac macro-tissues are stably positioned at the top of elastomeric pillars, which enabled measurement of pillar deflection and, hence, contractile force over time (FIG. 9,10). After platform assembly, a sacrificial gelatin layer is first added to support the filament during bioprinting. After bioprinting is completed, fibrinogen present within cardiac filaments is subsequently polymerized by adding 4° C., thrombin-rich media followed by incubation at 37° C. As the temperature increases, the gelatin within the ECM of the bioprinted cardiac tissue melts, facilitating thrombin diffusion and fibrin polymerization. During polymerization, the sacrificial gelatin layer also melts and is subsequently removed, resulting in an aligned, cellularly dense (>200⁶ cell/mL), contractile cardiac filament suspended between silicone pillars (FIG. 3A, FIGS. 11A-11C).

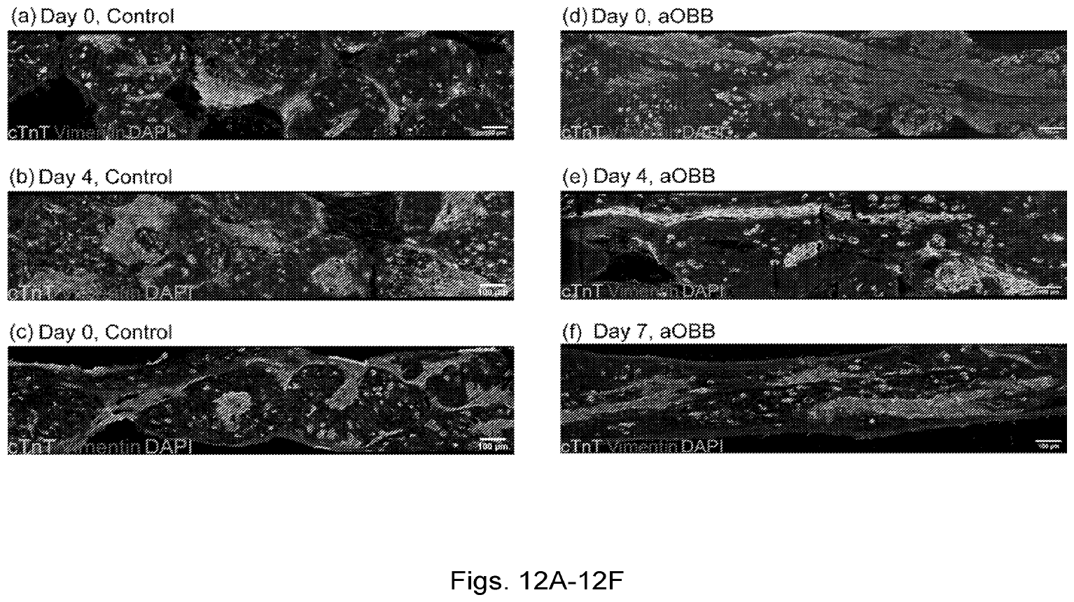

Immediately after bioprinting, the cardiac macro-tissues composed of aOBBs exhibited a preferred cellular alignment along the printing direction that was roughly three-fold higher than the spheroid-based controls (FIGS. 3B-3D). The inventors attributed this preferential alignment solely to the shear-induced orientation of cellularly-aligned aOBBs. However, after 7 days in culture, dramatic cellular reorientation in both types of bioprinted cardiac macro-tissues was observed (FIG. 3E). This analysis was conducted using confocal images of whole mount, stained cardiac macro-tissues, that largely depict the cells on the surface or periphery. However, cryo-sectioned slices, which are representative of the tissue mid-plane, revealed far less cellular reorientation (FIGS. 3F, 3G, 12A-12F). The inventors hypothesized that cells on the periphery of these cardiac macro-tissues can re-orient parallel to passive stress lines that arise due to cellular compaction, while reorientation in the tissue core is hindered by surrounding cells and extracellular matrix.

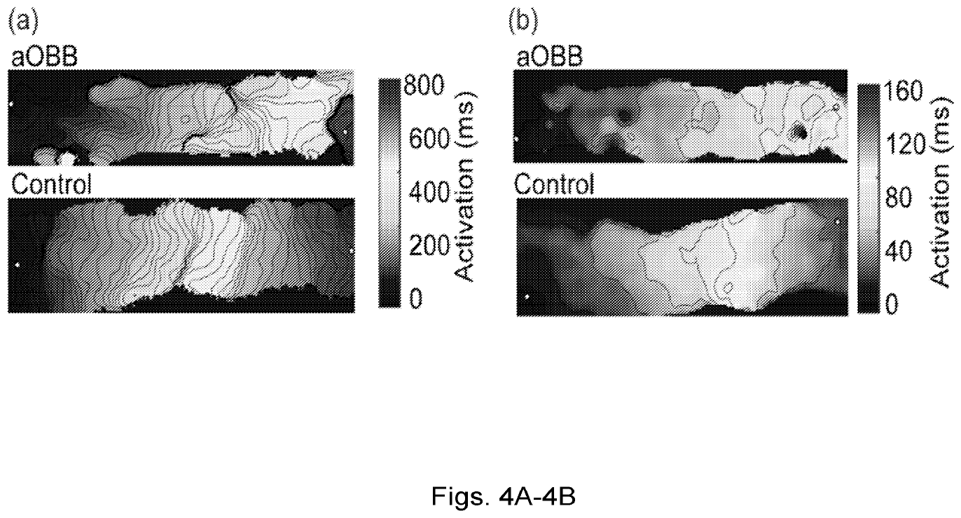

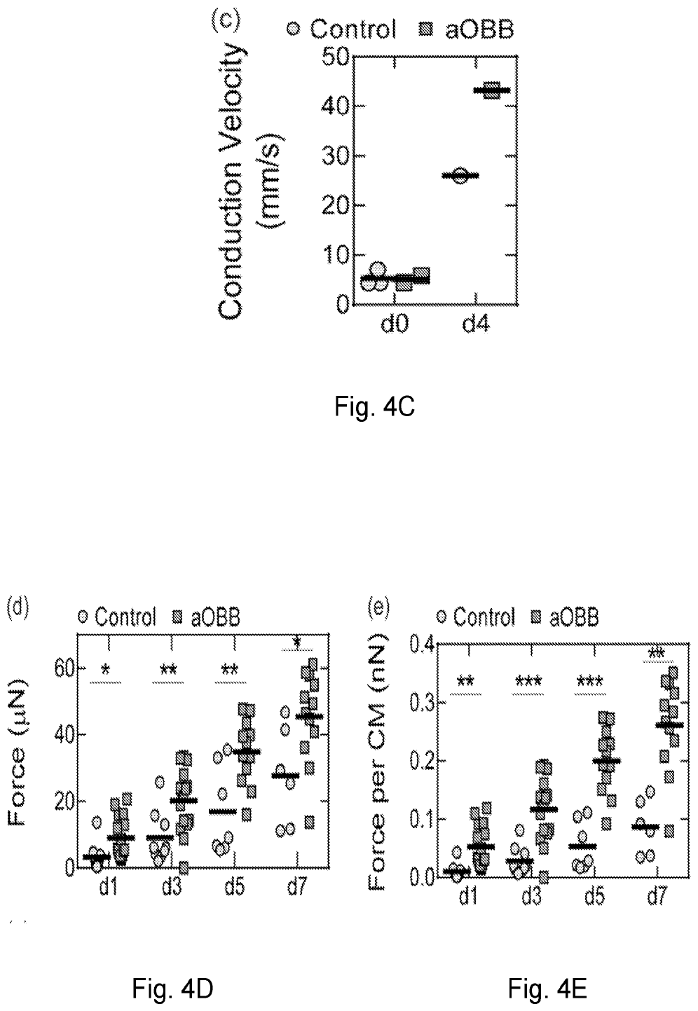

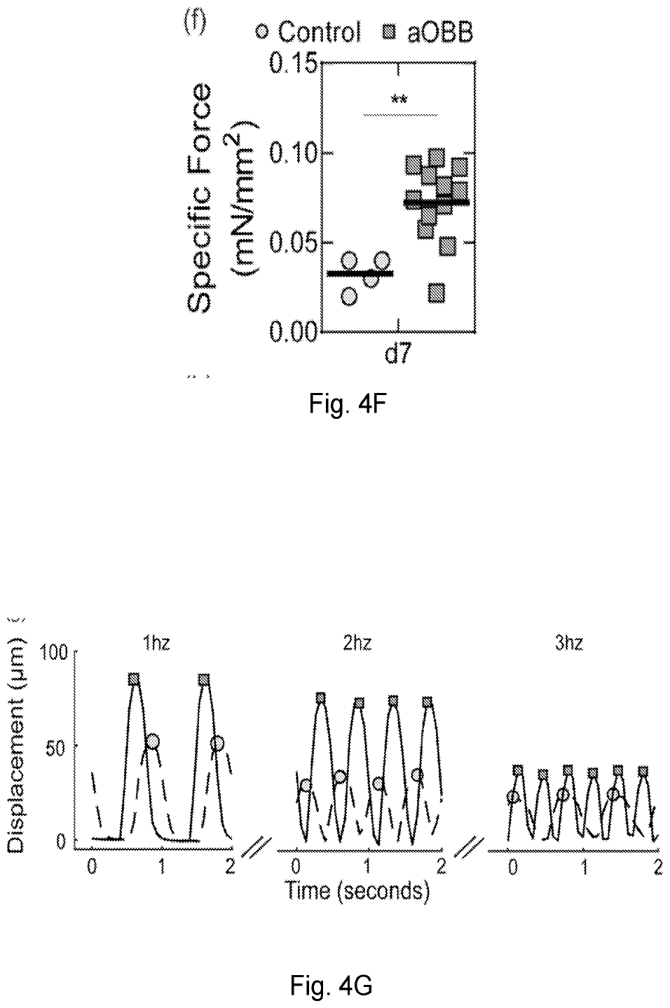

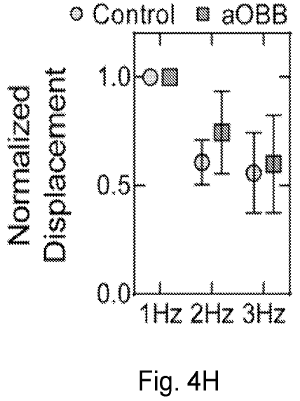

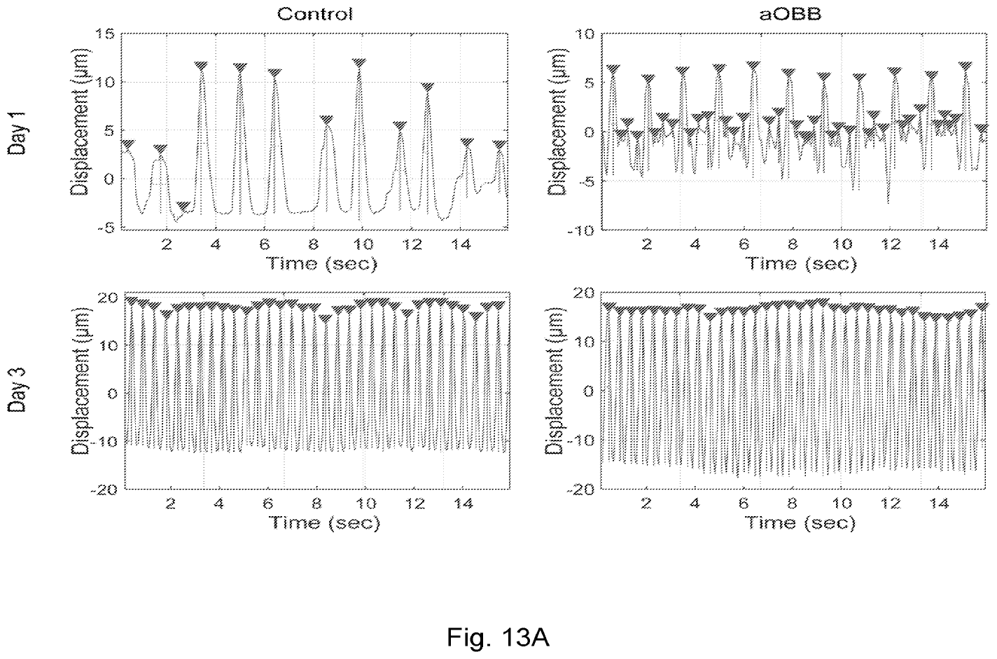

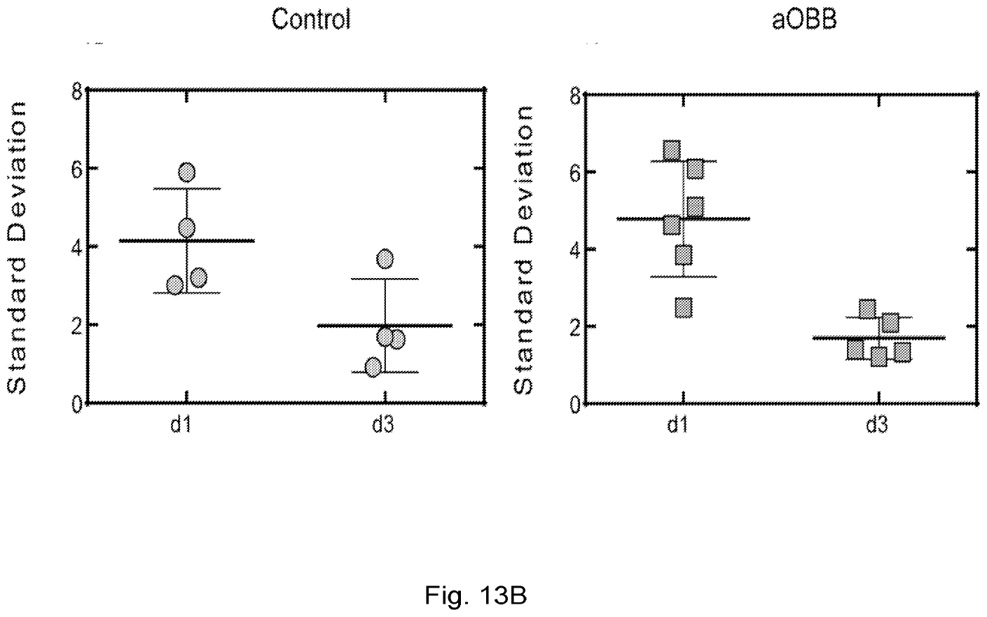

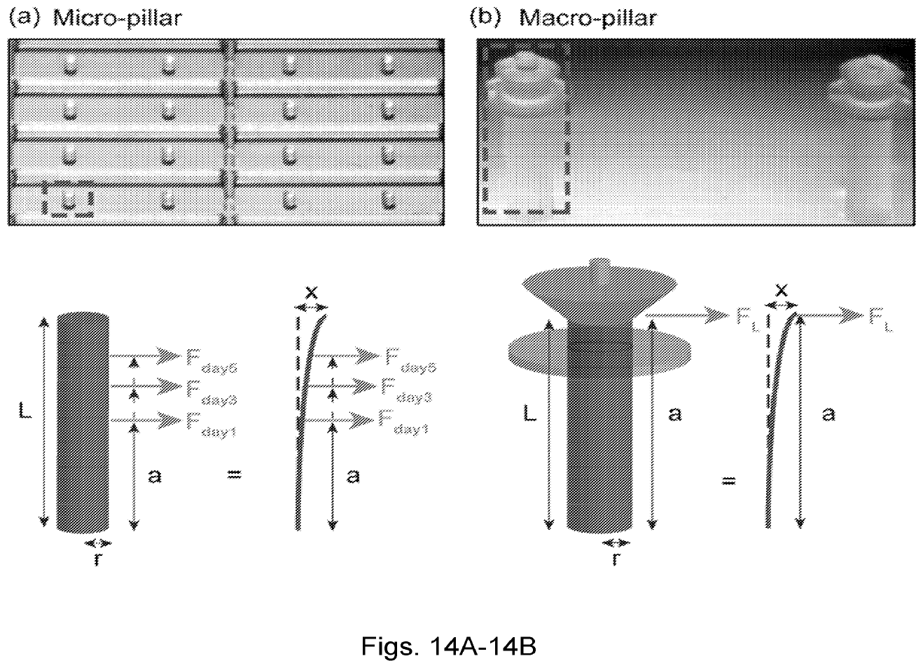

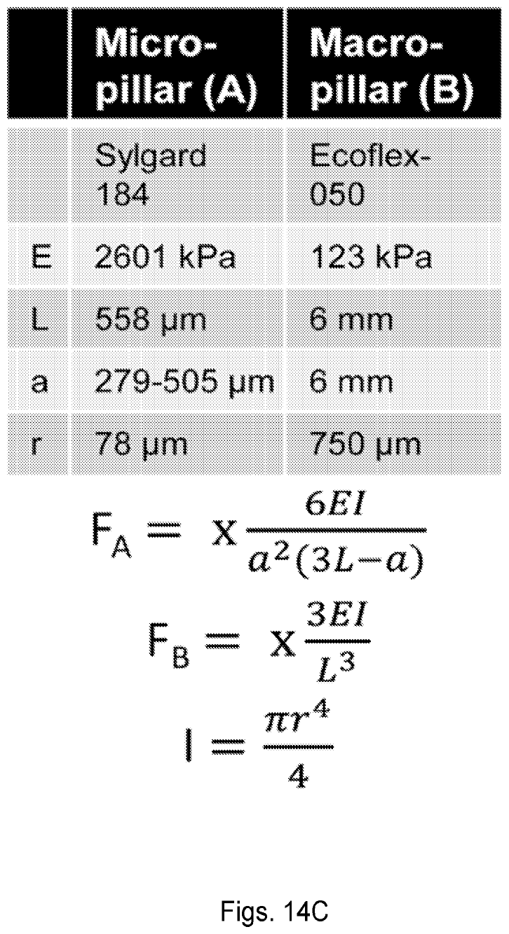

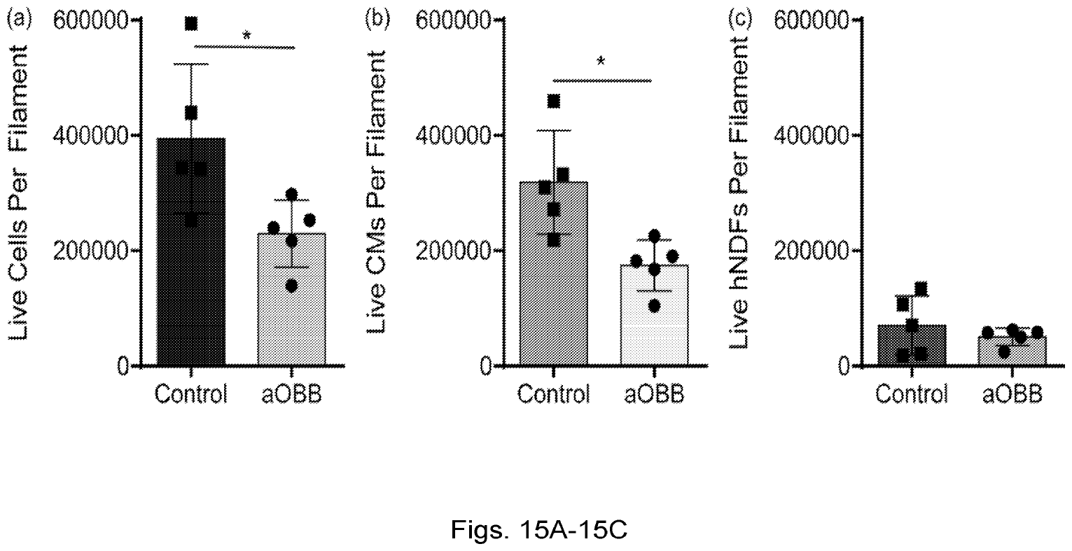

Next, in certain embodiments, the fusion of anisotropic and spheroidal OBBs into electromechanically synchronous cardiac tissue may be characterized. Fluorescent-based calcium imaging is used to visualize action potential propagation throughout bioprinted cardiac macro-tissues (Movies S6-S7). Day 1 after printing, the contractile wave of all cardiac macro-tissues exhibited a smooth propagation front (FIG. 4A). While the observed propagation in all macro-tissues demonstrated a distinct directionality, the conduction velocity (CV) was very slow (˜5 mm s⁻¹). Since the bioprinted macro-tissues are 1 cm in length, the slow CV resulted in asynchronous contraction of the discrete anisotropic and spheroidal OBBs. The incomplete recruitment of OBBs during each contractile cycle is represented by a wide range of recorded pillar displacement values on Day 1 (FIG. 13A-13B). As the macro-tissues matured, we observed fully synchronous contraction by Day 3 along with more uniform macro-pillar displacement values. As revealed by isochrone activation maps, CV increased dramatically in both the anisotropic and spheroid-based cardiac macro-tissues by Day 4 (FIGS. 4B, 4C). By applying Euler-Bernoulli beam theory, we extracted contractile force values from the macro-pillar deflections (FIGS. 14A-14C). Importantly, we find that cardiac macro-tissues composed of aOBBs generate a higher contractile force compared to spheroid-based controls (FIG. 4D). To further highlight the functional effect of directing cellular alignment, we sought to normalize macro-tissue forces per cardiomyocyte. As anisotropic particles are unable to compact as densely compared to spheroids,³⁸ we quantified the number of cardiomyocytes within each type of macro-tissue by flow cytometry (FIGS. 15A-15C). Immediately after printing, aOBB-based macro-tissues contain roughly 50% fewer cardiomyocytes compared to spheroid-based controls. Since iPSC-CMs are known to have low proliferation rates,³⁹ we normalized the measured force across all time points by the number of cardiomyocytes within each type of bioprinted tissue on Day 0. Based on this normalization, we find that the aligned cardiomyocytes within the aOBB-based macro-tissues exhibit a three-fold higher contractile force compared to the less aligned cardiomyocytes in spheroid-based controls (FIG. 4E). We also normalized force by measuring tissue stress. On Day 7 after bioprinting, we observed a two-fold higher stress value in the aligned cardiac macro-tissues composed of aOBBs compared to spheroid-based controls (FIG. 4F). To further assess their functional capacity, we used electrical field stimulation to pace these aligned cardiac macro-tissues at frequencies up to 3 Hz (Movie S5). While both types of cardiac macro-tissues could maintain electrical pacing at 1-2 Hz, only the aligned cardiac macro-tissues composed of aOBBs could maintain electrical pacing at 3 Hz (FIG. 4G). In both cases, a negative force-frequency relationship is observed (FIG. 4H), which is consistent with observations from other stem cell-derived cardiac tissues at similar time points.⁴⁰

In summary, described herein is a scalable biofabrication method to generate stem cell-derived, engineered cardiac tissue with high cellular density and programmed alignment. The bioinks composed of large quantities of aOBBs that were pre-assembled on micro-pillar arrays were created. Then it was demonstrated that these aOBBs could be programmably aligned along the printing direction. Finally, the inventors showed that by guiding aOBB orientation during print, they could also direct cellular alignment. Importantly and surprisingly, this programmed cellular alignment resulted in enhanced contractile function when compared to spheroid-based controls. This advance represents an important step towards our ultimate goal of fabricating aligned, vascularized cardiac tissues for therapeutic use.

In certain embodiments, potentially matching engineered tissue architecture with patient-specific defects as well as studying increasingly important metrics of cardiac function such as torsion and twist mechanics may be contemplated.

In one aspect of any of the embodiments, described herein is a tissue or tissue construct prepared by the method described herein, aligned at the aOBB scale.In one aspect of any of the embodiments, described herein is a tissue or tissue construct prepared by the method described herein, aligned at the aOBB, cellular, and sarcomeric length scales.

In some embodiments of any of the aspects, the aOBBS have isotropic cellular alignment. In some embodiments of any of the aspects, the aOBBs have anisotropic cellular alignment.

In one aspect of any of the embodiments, described herein is the use of a tissue or tissue construct as described herein, or produced by the methods described herein, in food production. In one aspect of any of the embodiments, described herein is a method of food production, comprising culturing a tissue or tissue construct as described herein, or according to the methods described herein. In embodiments relating to food production, the cells can comprise myocytes, e.g., cardiomyocytes or smooth muscle cells.

In one aspect of any of the embodiments, described herein is the use of a tissue or tissue construct as described herein, or produced by the methods described herein, in disease modeling. In one aspect of any of the embodiments, described herein is a method of disease modeling, comprising culturing a tissue or tissue construct as described herein, or according to the methods described herein. In embodiments relating to disease modeling, the cells can comprise diseased cells, e.g., cells comprising a mutation associated with a disease (e.g., a oncogene mutation or the like). In some embodiments, the disease is a cardiac disease. In embodiments relating to cardiac disease modeling, the cells can comprise myocytes, e.g., cardiomyocytes or smooth muscle cells.

In one aspect of any of the embodiments, described herein is the use of a tissue or tissue construct as described herein, or produced by the methods described herein, in drug toxicity studies or drug screening. In one aspect of any of the embodiments, described herein is a method of drug toxicity studies or drug screening production, comprising culturing a tissue or tissue construct as described herein, or according to the methods described herein. In some embodiments, the tissue or tissue construct is contacted with a candidate drug. In some embodiments, a marker or activity of the tissue or tissue construct is measured after being contacted with the candidate drug. In some embodiments, the cells can comprise diseased cells, e.g., cells comprising a mutation associated with a disease (e.g., a oncogene mutation or the like).

In one aspect of any of the embodiments, described herein is the use of a tissue or tissue construct as described herein, or produced by the methods described herein, in cardiac tissue replacement. In one aspect of any of the embodiments, described herein is a method of cardiac tissue replacement, comprising administering to a subject or implanting in a subject a tissue or tissue construct as described herein, or produced according to the methods described herein.

In one aspect of any of the embodiments, described herein is a bioink material comprising:
a carrier fluid, and
a plurality of anisotropic organ building blocks (aOBBs), each of the plurality of aOBBs comprising:
   at least one polymer; and
   at least one cell; and
wherein at the bioink material comprising a plurality of aligned aOBBs.

In one aspect of any of the embodiments, described herein is a bioink material comprising:
a carrier fluid, and
a plurality of anisotropic organ building blocks (aOBBs), each of the plurality of aOBBs comprising:
   at least one polymer; and
   at least one cell;
wherein the aOBBs are capable of anisotropically aligning along a flow direction when the bioink is flowed or is extruded from a three-dimensional printer or additive manufacturing system.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a condition. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a tissue or tissue structure as disclosed herein into a subject by a method or route which results in at least partial delivery of the tissue or tissue structure at a desired site. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, one element with another element. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Thus, for example, reference to “a protein” includes a plurality of such proteins and reference to “the progenitor cell” includes reference to one or more progenitor cells known to those skilled in the art, and so forth.Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Although methods and materials similar or equivalent to those described herein can be used in the practice of the disclosed methods and compositions, the exemplary methods, compositions, devices and materials are described herein. Definitions of common terms in molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:

1. A method of forming a tissue or tissue construct having arbitrarily programmed alignment, comprising:
extruding a bioink material through a nozzle onto a support to form a structure of the bioink material, the bioink material comprising:
a carrier fluid, and
anisotropic organ building blocks (aOBBs), the aOBBs comprising extracellular matrix material (ECM) and cellularly aligned cells, wherein at least some of the aOBBs align along a direction of extrusion in the structure of the bioink material; and
polymerizing the structure of the bioink material,
thereby forming the tissue or tissue construct having arbitrarily programmed alignment.

2. The method of paragraph 1, wherein the aOBBs comprise functional cells, stromal cells, or a mixture of functional and stromal cells.

3. The method of any of paragraphs 1 to 2, wherein the aOBBs comprise contractile induced pluripotent stem cell derived cardiomyocytes (iPSC-CMs) and stromal, human neonatal dermal fibroblasts (hNDF).

4. The method of paragraph 3, wherein the aOBBs comprise primary cardiomyocytes or embryonic stem cell derived cardiomyocytes (ESC-CM).

5. The method of any of paragraph 1 to 4, wherein the aOBBs further comprise support cells selected from the group consisting of primary fibroblasts, mesenchymal stem cells, epicardial derived cells, and a combination thereof.

6. The method of paragraph 1, wherein the aOBBs comprise contractile skeletal muscle cells (either primary or stem cell derived), mesenchymal stem cells (as stromal cells), neurons, endothelial cells, smooth-muscle cells, and fibroblasts.

7. The method of any of paragraphs 1 to 6, wherein the ECM comprises a blend of collagen and fibrin.

8. The method of any of paragraphs 1 to 7, wherein the ECM comprises any combination of biological polymers (collagen, fibrin, matrigel, hyaluornic acid, silk, alginate, chitosan) or synthetic materials (polyglycolic acid (PGA), poly(L)-lactic acid (PLA), poly(DL) glycolate (PLGA), and polyvinyl alcohol and their derivatives).

9. The method of any of paragraphs 1 to 8, further comprising providing the bioink material.

10. The method of paragraph 9, wherein the step of providing the bioink material comprises:
culturing the aOBBs within a pillar array;
harvesting the aOBBs from the pillar array by manual pipetting or by degradation of the pillar array;
resuspending the aOBBs in the carrier fluid;
compacting the aOBBs to achieve high volume fractions (i.e., cellular densities) to form the bioink material; and
adjusting rheology of the bioink material by modulating temperature.

11. The method of paragraph 10, wherein the carrier fluid comprises processed gelatin, fibrinogen and cell media.

12. The method of any of paragraphs 1 to 11, wherein the cellular density within the bioink is 10-500e6 cell/mL.

13. The method of any of paragraphs 1 to 12, wherein the support is a glass, plastic, or a biological hydrogel or matrix.

14. The method of any of paragraphs 1 to 13, wherein the cellular alignment forms a tissue pattern(s) selected from the group consisting of: linear, chevron, spiral pattern, and a combination of patterns.

15. The method of any of paragraphs 1 to 14, further comprising vascularizing the tissue or tissue construct.

16. A tissue or tissue construct prepared by the method of any of paragraphs 1 to 15 aligned at the aOBB, cellular, and sarcomeric length scales.

17. The tissue or tissue construct of paragraph 16, wherein the aOBBS have isotropic cellular alignment.

18. The tissue or tissue construct of paragraph 16, wherein the aOBBs have anisotropic cellular alignment.

19. Use of a tissue or tissue construct produced by the methods of any of paragraphs 1 to 15 in disease modeling.

20. Use of a tissue or tissue construct produced by the methods of any of paragraphs 1 to 15 in cardiac disease modeling.

21. Use of a tissue or tissue construct produced by the methods of any of paragraphs 1 to 15 in drug toxicity studies.

22. Use of a tissue or tissue construct produced by the methods of any of paragraphs 1 to 15 in drug screening applications.

23. Use of a tissue or tissue construct produced by the methods of any of paragraphs 1 to 15 as cardiac tissue for replacement of heart in regenerative medicine.

24. A bioink material for use with a three-dimensional printer or an additive manufacturing system, the bioink comprising:
a carrier fluid, and
anisotropic organ building blocks (aOBBs) comprising extracellular matrix material (ECM) and cellularly aligned cells, wherein at least some of the aOBBs are capable of anisotropically align along an extrusion direction when the bioink is extruded from a three-dimensional printer or additive manufacturing system.

25. The bioink of paragraph 25, wherein the cellular density within the bioink is 10-500e6 cell/mL.

In some embodiments, the present technology may be defined in any of the following numbered paragraphs:

1. A method comprising applying a bioink material onto a support, the bioink material comprising:
a carrier fluid, and
a plurality of anisotropic organ building blocks (aOBBs), each of the plurality of aOBBs comprising:
   at least one polymer; and
   at least one cell;
wherein the applied bioink material comprises a plurality of aligned aOBBs.

2. The method of any one of the preceding paragraphs, wherein at least 90% of the plurality of aligned aOBBs have an aOBB anisotropic axis that is within +/−60 degrees of a mean aoBB anisotropic axis of the plurality of aligned aoBBs.

3. The method of any one of the preceding paragraphs, wherein at least 60% of the plurality of aligned aOBBs have an aOBB anisotropic axis that is within +/−30 degrees of a mean aoBB anisotropic axis of the plurality of aligned aoBBs.

4. The method of any one of the preceding paragraphs, wherein at least 60% of the plurality of aligned aOBBs have an aOBB anisotropic axis that is within +/−10 degrees of a mean aoBB anisotropic axis of the plurality of aligned aoBBs.

5. The method of any one of the preceding paragraphs, wherein at least 60% of the plurality of aligned aOBBs have an aOBB anisotropic axis that is within 1 standard deviation of a mean aoBB anisotropic axis of the plurality of aligned aoBBs.

6. The method of any one of the preceding paragraphs, wherein at least 90% of the plurality of aligned aOBBs have an aOBB anisotropic axis that is within 2 standard deviations of a mean aoBB anisotropic axis of the plurality of aligned aoBBs.

7. The method of any one of the preceding paragraphs, wherein at least 90% of the plurality of aligned aOBBs have an aOBB anisotropic axis that is within +/−60 degrees of an anisotropic axis of the applied bioink material.

8. The method of any one of the preceding paragraphs, wherein at least 60% of the plurality of aligned aOBBs have an aOBB anisotropic axis that is within +/−30 degrees of an anisotropic axis of the applied bioink material.

9. The method of any one of the preceding paragraphs, wherein at least 60% of the plurality of aligned aOBBs have an aOBB anisotropic axis that is within +/−10 degrees of an anisotropic axis of the applied bioink material.

10. The method of any one of the preceding paragraphs, further comprising flowing the bioink material prior to or concurrently with the applying step.

11. The method of paragraph 10, wherein the flowing comprises exposing the bioink material to shear stress.

12. The method of any one of the preceding paragraphs, wherein the applying comprises extruding, flowing, injecting the bioink material onto the support.

13. The method of any one of the preceding paragraphs, wherein the applying comprises flowing the bioink material onto the support.

14. The method of any one of the preceding paragraphs, wherein the applying comprises extruding the bioink material onto the support.

15. The method of any one of the preceding paragraphs, wherein the applying comprises moving the bioink material through a nozzle, die, channel, or tube.

16. The method of any one of the preceding paragraphs, wherein the applying comprises moving the bioink material through a nozzle.

17. The method of any one of the preceding paragraphs, wherein the applied bioink material is in the form of one or more filaments.

18. The method of any one of the preceding paragraphs, further comprising polymerizing the bioink material after the applying step.

19. The method of any one of the preceding paragraphs, wherein the at least one polymer comprises at least one extracellular matrix material.

20. The method of any one of the preceding paragraphs, wherein the at least one polymer comprises at least one synthetic polymer.

21. The method of any one of the preceding paragraphs, wherein the at least one polymer comprises at least one biomaterial.

22. The method of any one of the preceding paragraphs, wherein the at least one polymer comprises at least one material selected from the group consisting of:
collagen, fibrin, MATRIGEL, hyaluronic acid, silk, alginate, chitosan, alginate, gelatin, heparin, chondroitin sulfate, polyethylene glycol (PEG), polyethylene glycol diacrylate (PEGDA), polyglycolic acid PGA), poly(l)-lactic acid (PLA), poly(L) glycolate PLGA),and polyvinyl alcohol and their derivatives.

23. The method of any one of the preceding paragraphs, wherein the at least one polymer comprises at least one material selected from the group consisting of:
fibrin, alginate, chitosan, alginate, gelatin, heparin, chondroitin sulfate, polyethylene glycol (PEG), and polyethylene glycol diacrylate (PEGDA).

24. The method of any one of the preceding paragraphs, wherein the at least one polymer comprises or consists of collagen and fibrin.

25. The method of any one of the preceding paragraphs, wherein the at least one polymer comprises or consists of alginate.

26. The method of any one of the preceding paragraphs, wherein the at least one polymer comprises or consists of alginate and PEGDA; gelatin and alginate; gelatin and collagen; or gelatin and fibrin.

27. The method of any one of the preceding paragraphs, wherein the at least one cell comprises functional cells, stromal cells, or a mixture of functional and stromal cells.

28. The method of any one of the preceding paragraphs, wherein the at least one cell comprises cardiomyocytes and stromal cells.

29. The method of paragraph 28, wherein the cardiomyoctes comprise contractile induced pluripotent stem cell derived cardiomyocytes (iPSC-CMs), primary cardiomyocytes, and/or or embryonic stem cell derived cardiomyocytes (ESC-CM).

30. The method of any one of paragraphs 27-29, wherein the stromal cells comprise fibroblasts.

31. The method of paragraph 30, wherein the fibroblasts comprise human neonatal dermal fibroblasts (hNDF).

32. The method of any one of the preceding paragraphs, wherein the at least one cell further comprises one or more support cells selected from the group consisting of:
primary fibroblasts, mesenchymal stem cells, epicardial derived cells, and a combination thereof.

33. The method of any one of the preceding paragraphs, wherein the at least one cell comprises one or more of:
contractile skeletal muscle cells (either primary or stem cell derived), mesenchymal stem cells (e.g., stromal cells), neurons, endothelial cells, smooth-muscle cells, and fibroblasts.

34. The method of any one of the preceding paragraphs, wherein the at least one cell is a plurality of cells.

35. The method of paragraph 34, wherein the plurality of cells of an aOBB are cellularly aligned.

36. The method of paragraph 35, wherein at least 90% of the plurality of cells have a cellular anisotropic axis that is within +/−60 degrees of a mean cellular anisotropic axis of the plurality of cells of the aOBB.

37. The method of any one of paragraphs 34-36, wherein at least 60% of the plurality of cells have a cellular anisotropic axis that is within +/−30 degrees of a mean cellular anisotropic axis of the plurality of cells of the aOBB.

38. The method of any one of paragraphs 34-37, wherein at least 60% of the plurality of cells have a cellular anisotropic axis that is within +/−10 degrees of a mean cellular anisotropic axis of the plurality of cells of the aOBB.

39. The method of any one of paragraphs 34-38, wherein at least 60% of the plurality of cells have a cellular anisotropic axis that is within 1 standard deviation of a mean cellular anisotropic axis of the plurality of cells of the aOBB.

40. The method of any one of paragraphs 34-39, wherein at least 90% of the plurality of cells have a cellular anisotropic axis that is within 2 standard deviations of a mean cellular anisotropic axis of the plurality of cells of the aOBB.

41. The method of any one of the preceding paragraphs, wherein the bioink material comprises 10-1000e6 cells/mL.

42. The method of any one of the preceding paragraphs, wherein the plurality of aOBBs are at least 5% volume of the bioink material.

43. The method of any one of the preceding paragraphs, wherein the carrier fluid comprises one or more of processed gelatin, fibrinogen, collagen, alginate, RGD-modified alginate, fibrin, and cell media.

44. The method of any one of the preceding paragraphs, wherein the carrier fluid comprises one or more of processed gelatin, fibrinogen, and cell media.

45. The method of any one of the preceding paragraphs, further comprising compacting the bioink material before the applying step.

46. The method of paragraph 45, wherein the compacting comprises centrifugation, capillary flow to increase the aOBB density in the bioink material, or evaporation.

47. The method of any one of the preceding paragraphs, wherein the support is a glass support, a plastic support, or a biological hydrogel or matrix.

48. The method of any one of the preceding paragraphs, wherein the support does not comprise an anisotropic scaffold.

49. The method of any one of the preceding paragraphs, further comprising a first step of providing the bioink material, comprising:
culturing the aOBBs;
contacting the aOBBs with the carrier fluid; and
compacting the aOBBs.

50. The method of any one of the preceding paragraphs, further comprising a first step of providing the bioink material, comprising:
culturing the aOBBs within a pillar array;
contacting the aOBBs with at least one ROCK inhibitor;
harvesting the aOBBs from the pillar array by manual pipetting or by degradation of the pillar array;
resuspending the aOBBs in the carrier fluid; and
compacting the aOBBs.

51. The method of any one of the preceding paragraphs, further comprising a first step of providing the bioink material, comprising:
culturing the aOBBs within a pillar array;
harvesting the aOBBs from the pillar array by manual pipetting or by degradation of the pillar array;
resuspending the aOBBs in the carrier fluid;
compacting the aOBBs; and
adjusting rheology of the bioink material by modulating temperature.

52. The method of any one of the preceding paragraphs, wherein the applied bioink forms a pattern(s) selected from the group consisting of: linear, chevron, spiral pattern, and a combination of patterns.

53. The method of any one of the preceding paragraphs, further comprising culturing the applied bioink to form a tissue or tissue construct.

54. The method of paragraph 53, further comprising vascularizing the tissue or tissue construct.

55. A tissue or tissue construct prepared by the method of any of paragraphs 1 to 54 aligned at the aOBB, cellular, and sarcomeric length scales.

56. The tissue or tissue construct of paragraph 55, wherein the aOBBS have isotropic cellular alignment.

57. The tissue or tissue construct of paragraph 55, wherein the aOBBs have anisotropic cellular alignment.

58. Use of a tissue or tissue construct produced by the methods of any of paragraphs 1 to 54 in food production.

59. Use of a tissue or tissue construct produced by the methods of any of paragraphs 1 to 54 in disease modeling.

60. Use of a tissue or tissue construct produced by the methods of any of paragraphs 1 to 54 in cardiac disease modeling.

61. Use of a tissue or tissue construct produced by the methods of any of paragraphs 1 to 54 in drug toxicity studies.

62. Use of a tissue or tissue construct produced by the methods of any of paragraphs 1 to 54 in drug screening applications.

63. Use of a tissue or tissue construct produced by the methods of any of paragraphs 1 to 54 as cardiac tissue for replacement of heart in regenerative medicine.

64. A bioink material comprising:
a carrier fluid, and
a plurality of anisotropic organ building blocks (aOBBs), each of the plurality of aOBBs comprising:
   at least one polymer; and
   at least one cell;
wherein at least some of the aOBBs are capable of anisotropically aligning along a flow direction when the bioink is flowed or is extruded from a three-dimensional printer or additive manufacturing system.

65. The bioink of paragraph 64, wherein the cellular density within the bioink is 10-500e6 cell/mL.

66. The bioink of any one of paragraphs 64-65, wherein at least 90% of the plurality of aligned aOBBs have an aOBB anisotropic axis that is within +/−60 degrees of a mean aoBB anisotropic axis of the plurality of aligned aoBBs.

67. The bioink of any one of paragraphs 64-66, wherein at least 60% of the plurality of aligned aOBBs have an aOBB anisotropic axis that is within +/−30 degrees of a mean aoBB anisotropic axis of the plurality of aligned aoBBs.

68. The bioink of any one of paragraphs 64-67, wherein at least 60% of the plurality of aligned aOBBs have an aOBB anisotropic axis that is within +/−10 degrees of a mean aoBB anisotropic axis of the plurality of aligned aoBBs.

69. The bioink of any one of paragraphs 64-68, wherein at least 60% of the plurality of aligned aOBBs have an aOBB anisotropic axis that is within 1 standard deviation of a mean aoBB anisotropic axis of the plurality of aligned aoBBs.

70. The bioink of any one of paragraphs 64-69, wherein at least 90% of the plurality of aligned aOBBs have an aOBB anisotropic axis that is within 2 standard deviations of a mean aoBB anisotropic axis of the plurality of aligned aoBBs.

71. The bioink of any one of paragraphs 64-70, wherein at least 90% of the plurality of aligned aOBBs have an aOBB anisotropic axis that is within +/−60 degrees of an anisotropic axis of the applied bioink material.

72. The bioink of any one of paragraphs 64-71, wherein at least 60% of the plurality of aligned aOBBs have an aOBB anisotropic axis that is within +/−30 degrees of an anisotropic axis of the applied bioink material.

73. The bioink of any one of paragraphs 64-72, wherein at least 60% of the plurality of aligned aOBBs have an aOBB anisotropic axis that is within +/−10 degrees of an anisotropic axis of the applied bioink material.

74. The bioink of any one of paragraphs 64-73, wherein the at least one polymer comprises at least one extracellular matrix material.

75. The bioink of any one of paragraphs 64-74, wherein the at least one polymer comprises at least one synthetic polymer.

76. The bioink of any one of paragraphs 64-75, wherein the at least one polymer comprises at least one biomaterial.

77. The bioink of any one of paragraphs 64-76, wherein the at least one polymer comprises at least one material selected from the group consisting of:
collagen, fibrin, MATRIGEL, hyaluronic acid, silk, alginate, chitosan, alginate, gelatin, heparin, chondroitin sulfate, polyethylene glycol (PEG), polyethylene glycol diacrylate (PEGDA), polyglycolic acid PGA), poly(l)-lactic acid (PLA), poly(L) glycolate PLGA),and polyvinyl alcohol and their derivatives.

78. The bioink of any one of paragraphs 64-77, wherein the at least one polymer comprises at least one material selected from the group consisting of:
fibrin, alginate, chitosan, alginate, gelatin, heparin, chondroitin sulfate, polyethylene glycol (PEG), and polyethylene glycol diacrylate (PEGDA).

79. The bioink of any one of paragraphs 64-78, wherein the at least one polymer comprises or consists of collagen and fibrin.

80. The bioink of any one of paragraphs 64-79, wherein the at least one polymer comprises or consists of alginate.

81. The bioink of any one of paragraphs 64-80, wherein the at least one polymer comprises or consists of alginate and PEGDA; gelatin and alginate; gelatin and collagen; or gelatin and fibrin.

82. The bioink of any one of paragraphs 64-81, wherein the at least one cell comprises functional cells, stromal cells, or a mixture of functional and stromal cells.

83. The bioink of any one of paragraphs 64-82, wherein the at least one cell comprises cardiomyocytes and stromal cells.

84. The bioink of any paragraph 83, wherein the cardiomyoctes comprise contractile induced pluripotent stem cell derived cardiomyocytes (iPSC-CMs), primary cardiomyocytes, and/or or embryonic stem cell derived cardiomyocytes (ESC-CM).

85. The bioink of any one of paragraphs 64-84, wherein the stromal cells comprise fibroblasts.

86. The bioink of paragraph 85, wherein the fibroblasts comprise human neonatal dermal fibroblasts (hNDF).

87. The bioink of any one of paragraphs 64-86, wherein the at least one cell further comprises one or more support cells selected from the group consisting of:
primary fibroblasts, mesenchymal stem cells, epicardial derived cells, and a combination thereof.

88. The bioink of any one of paragraphs 64-87, wherein the at least one cell comprises one or more of:
contractile skeletal muscle cells (either primary or stem cell derived), mesenchymal stem cells (e.g., stromal cells), neurons, endothelial cells, smooth-muscle cells, and fibroblasts.

89. The bioink of any one of paragraphs 64-88, wherein the at least one cell is a plurality of cells.

90. The bioink of paragraph 89, wherein the plurality of cells of an aOBB are cellularly aligned.

91. The bioink of paragraph 90, wherein at least 90% of the plurality of cells have a cellular anisotropic axis that is within +/−60 degrees of a mean cellular anisotropic axis of the plurality of cells of the aOBB.

92. The bioink of any one of paragraphs 90-91, wherein at least 60% of the plurality of cells have a cellular anisotropic axis that is within +/−30 degrees of a mean cellular anisotropic axis of the plurality of cells of the aOBB.

93. The bioink of any one of paragraphs 90-92, wherein at least 60% of the plurality of cells have a cellular anisotropic axis that is within +/−10 degrees of a mean cellular anisotropic axis of the plurality of cells of the aOBB.

94. The bioink of any one of paragraphs 90-93, wherein at least 60% of the plurality of cells have a cellular anisotropic axis that is within 1 standard deviation of a mean cellular anisotropic axis of the plurality of cells of the aOBB.

95. The bioink of any one of paragraphs 90-94, wherein at least 90% of the plurality of cells have a cellular anisotropic axis that is within 2 standard deviations of a mean cellular anisotropic axis of the plurality of cells of the aOBB.

96. The bioink of any one of paragraphs 64-95, wherein the plurality of aOBBs are at least 50% volume of the bioink material.

97. The bioink of any one of paragraphs 64-96, wherein the carrier fluid comprises one or more of processed gelatin, fibrinogen, collagen, alginate, RGD-modified alginate, fibrin, and cell media.

98. The bioink of any one of paragraphs 64-97, wherein the carrier fluid comprises one or more of processed gelatin, fibrinogen, and cell media.

The technology described herein is further illustrated by the following examples which in no way should be construed as being further limiting.

### EXAMPLES

### Example 1

### Cells, Embryoid Bodies, and Spheroids:

Inventors obtained BJFF hiPS cell line from Dr. Jain at Washington University. This cell line was maintained and cells used to create hiPSC-derived cardiomyocytes following the protocols described in prior work.⁴¹ Briefly, hiPSCs were cultured daily in supplemented mTeSR media (STEMCELL Technologies) at 37° C./5.5% CO₂. hiPSCs were passaged every 4 days (when 80% confluency was reached) or used for embryoid body (EB) formation and cardiomyocyte differentiation. On passage days, the cells were rinsed in PBS without Ca²⁺ or Mg²⁺ (PBS −/−) and then rinsed with enzyme-free dissociation reagent ReleSR (STEMCELL Technologies). After immediate aspiration of ReleSR, hiPSCs were incubated at 37° C./5.5% CO₂ for 8 min. Following this short incubation, the cells were gently resuspended in mTeSR media to dislodge hiPSCs as cell clusters and transferred to T225 culture flasks freshly coated with Matrigel® (Corning) at a split ratio of 1:8. We also cultured hNDFs (Cascade Biologics) in Dulbecco's Modified Eagle Medium (DMEM, Gibco) supplemented with 1% penicillin-streptomycin and 10% fetal bovine serum (Gibco) at 37° C./5.5% CO₂. Culture media was replaced every 2 days. For passage days, hNDFs were rinsed in PBS (−/−), then incubated for 5 min with TrypLE cell disassociation reagent (ThermoFisher Scientific). Cells were then washed in supplemented DMEM and split at 1:10 culture ratio in T225 culture flasks (Falcon) once weekly (at 80% confluency) or were otherwise used for experiments. Cell viability prior to each experiment was quantified by AO/PI using an automated cell counter (Auto2000, Nexcelom).

Next, embryoid bodies (EBs) were generated following prior protocol as described below. Briefly, on Day −2 of differentiation, hiPSCs were transferred into aggrewell arrays to induce aggregation. The aggrewell arrays were first rinsed in isopropanol followed by autoclave-sterilization. Next, 2 mL of 0.2% Pluronic (Sigma) was added, followed by two rinses with DMEM/F12 (+HEPES and L-glutamine, GIBCO). 1 mL of PVA medium (mTeSR+ 4 mg/mL polyvinyl alcohol) and 10 μM of rho-associated coiled-coil forming kinase inhibitor (Y-27635, Biogems) was then added to each well. To prepare hiPSCs, the cells were rinsed with PBS (−/−) followed by a 12 min incubation with Gentle Cell dissociation reagent (STEMCELL Technologies) to dislodge growing culture colonies from T225 culture flasks. The cells were then resuspended in DMEM/F12 medium, transferred into a 50 mL conical tube, and centrifuged at 240 g for 5 min. The hiPSC pellet was resuspended in 12 mL PVA medium+10 μM Y-27635. hiPSCs were evenly distributed into 2 aggrewell arrays per each T225 culture flask. Finally, aggrewell plates with hiPSCs were centrifuged at 100 g for 3 min and then placed in the incubator to aggregate at 37° C./5.5% CO₂.

Finally, these hiPSC EBs were used to generate cardiac spheroids following a protocol that modulates Wnt/β-Catenin pathways (FIG. 5).⁴² Briefly, on Day 0 of differentiation, hiPSC media was removed from each T25 flask by rinsing and resuspending the EBs in 5 μM of WNT activator CHIR (CHIR 99021, Biogems) diluted in cardiac differentiation media (CDM) that consisted of basal RPMI-1640 (Gibco) supplemented with 2% B27/-insulin supplement (ThermoFischer Scientific). Fresh supplemented CDM media was added on Day 1. CHIR was removed on Day 2 by rinsing and resuspending the spheroids in CDM only. On Days 3 and 4, spheroids were cultured with 24M of WNT inhibitor iWR1(Biogems) in CDM. On Day 5 iWR1 was removed by rinsing and the spheroids were cultured in CDM only through Day 6. From Day 7 to Day 9, the spheroids were cultured in cardiac maturation medium [CMM, 2% B27 (ThermoFisher Scientific) in RPMI-1640]. Lastly, from Day 9 to 11, to metabolically purify the hiPSC-CMs, spheroids were cultured in RPMI without glucose (Gibco) supplemented with 2% B27 and 4 mM lactate (Sigma). All steps highlighted from Day 0 to Day 11 were performed with spheroids cultured in T25 flaks in an orbital shaker at 53 rpm and at 37° C./5.5% CO₂.

### Micro-Pillar Array Fabrication:

Micro-pillar arrays were used to generate cardiac or fibroblast aOBBs. To generate a scalable number of aOBBs (˜10,000 per experiment), polydimethylsiloxane (PDMS, Sylgard 184, Dow Corning) plates were created that contained 1050 micro-pillar arrays. Plates were designed to fit inside Nunc™ OmniTray™-well plates that are compatible with conventional imaging equipment. The first step in this fabrication process was to print a single rectangular mold that contained 175 micro-pillar arrays using a stereolithographic (SLA) 3D printer (Perfactory Aureues Plus, EnvisionTec) using their photo-curable resin HTM140 V2. To increase their mechanical strength, the SLA-printed rectangular molds were UV post-cured for 5 min (OmniCure, Excelitas). Prior to the UV exposure, the molds were heated on a hot-plate to 80° C., beyond the glass-transition temperature (T_{g}) of the cured resin. Next, the mold surface was covalently modified with fluorosilane (1H,1H,2H,2H-Perfluorooctyltriethoxysilane 97%, Oakwood Chemical) to ensure proper PDMS curing during the transfer molding process. Briefly, to prepare the device for salinization, it was first plasma treated with the following settings: 5 min plasma, 100% O₂, 100% power (Femto, Diener). After plasma treatment, the rectangular mold was immediately transferred to a vacuum chamber containing fluorosilane, where it remained under vacuum for a minimum of 2 h. The next step in the fabrication process was to generate reusable elastomeric negatives of these micro-pillar array molds. First, the six SLA-printed molds were placed in laser-cut acrylic holders designed to fit snugly into a Nunc™ 1-well plate (ThermoFischer Scientific). To generate a negative mold, a flexible and strong silicone (SortaClear37™, Smooth-On) was formulated at 1:1 (PartA:PartB) mass ratio with 2% (Part A+Part B) SloJo (SmoothOn) to increase pot-life and 10% (Part A) SilioneThinner (SmoothOn) to reduce viscosity. These components were mixed via centrifugation in a SpeedMixer (FlackTek Inc.) for 3 min at 2000 g. The silicone was then poured over the six (contiguous) SLA-printed molds and acrylic holder. The plate was then degassed in a vacuum chamber for 1 min and left overnight at room temperature to cure. The negative micro-pillar array plate was removed manually from the mold. To enable the subsequent silicone-on-silicone transfer molding process, the same fluorosilane surface modification was repeated for the negative micro-pillar array plate. In addition, after the plate was taken out of the vacuum, it was post-cured at 80° C. for 2 h. After the post-curing process, the negative plate was placed back into the Nunc™ 1-well plate. Finally. PDMS mixed at 10:1 (base:catalyst) mass ratio was poured into this negative mold, degassed for 5 min in a vacuum, and then polymerized for 3 h at 80° C. The final PDMS micro-pillar array plate was removed manually and autoclaved prior to use.

### Anisotropic OBBs:

To generate aOBBs, first, the PDMS micro-pillar array plates were prepared for cell seeding. Each PDMS plate was plasma treated for 1 min in 100% O₂ gas at full power (Femto, Diener) to increase hydrophilicity and wetting. Immediately after plasma treatment, anti-adhesion rinsing solution (STEMCELL Technologies) is added to each well, where it remained for >30 min at room temperature. After 30 min, we aspirated the rinsing solution and washed the plates twice with DMEM/F12 (+HEPES and L-glutamine, GIBCO).

After preparation of the PDMS micro-pillar array plates, the cardiac spheroids were dissociated into single cells. It is critical to dissociate cardiac spheroids using as soft an enzymatic solution as possible to increase viability of iPSC-CMs. In addition, all serological pipette tips should be treated with 3% bovine serum albumin (BSA, Sigma) to prevent adhesion and loss of cardiac spheroids. The first step in cardiac spheroid dissociation is to incubate spheroids in 1 mg/mL solution of CollagenaseB (Sigma) diluted in Earl's Balanced Salt Solution (EBSS, ThermoFischer). 10 mL of 1 mg/mL CollagenaseB solution is made per an estimated 20×10⁶ cells. The calcium concentration of CollagenaseB is adjusted to 5 mM for enhanced enzymatic activity. First, cardiac spheroids are aggregated via gravity. The supernatant is aspirated and the cardiac spheroids are resuspended in CollagenaseB solution. The suspension is transferred to ultra-low attachment 6 well plates (Corning) and incubated for 1 h at 37° C./5.5% CO₂, on an orbital shaker set to 53 revolutions per minute (RPM). After 1 h of incubation, the spheroids are aggregated in 50 mL tubes and diluted 1:1 in PBS without Ca²⁺ and Mg²⁺ (PBS −/−), to inactivate the calcium-dependent CollagenaseB. In addition, calcium is a co-factor for cell-cell binding and its sequestration enhances single cell dissociation. Each 50 mL tube is centrifuged at 240 g for 5 min and then washed in 50 mL PBS −/−.

The second step in dissociation is to enzymatically digest the cardiac spheroids using TrypLE™ Express (ThermoFischer). TrypLE™ Express solution is supplemented with 40 U/mL DNAseI (Worthington, Inc). Cardiac spheroids are aggregated and incubated in 1 mL TrypLE™ Expression solution at 37° C. for 15 min. Prior to enzymatic inactivation, the spheroids are mechanically dissociated by pipetting ˜20× with P1000 pipette. After mechanical dissociation, the enzymatic solution is inactivated by diluting the sample in DMEM/F12. After centrifuging at 240 g for 5 min, the supernatant is aspirated, and the pellet resuspended in DMEM/F12. To ensure accurate cell counting, cellular aggregates are filtered by pipetting the resuspension through a 70 μm filter. Viability and cellular density are quantified using Cellometer Auto 2000 Automated Cell Counter (Nexcelom). The single cell suspension is kept on ice. Next, a monolayer of hNDFs are enzymatically dissociated into single cells and counted. Briefly, the monolayer is washed in PBS without Ca²⁺ and Mg²⁺ (PBS −/−). These cells are incubated in TrypLE™ solution for 10 min at 37° C. After mechanical dissociation, the enzymatic solution is diluted in DMEM/F12 and centrifuged at 240 g for 5 min. After aspiration of the supernatant, the pellet is resuspended in DMEM/F12, and counted using a Cellometer Auto 2000 Automated Cell Counter. The single cell suspensions of iPSC-CMs and hDNFs are then combined in a 9:1 ratio iPSC-CMs:hNDFs. The corresponding volumes of each suspension are mixed and centrifuged at 240 g for 5 min and kept on ice.

Based upon the total concentration of iPSC-CMs, an appropriate volume of 1× rat tail collagen I (Corning) solution was prepared on ice. An appropriate volume of collagen I is calculated based upon a desired final concentration of 1.25 mg/mL. Then y/100 mL of 250 mM CaCl₂, ˜x/40 mL of 1 N NaOH, and y mL of collagen I are added, in order, to the basal DMEM high glucose media. After aspiration of the supernatant, the cellular pellet is resuspended in a volume of collagen gel that yielded a final concentration of 10⁶ cell/mL. Next, 250 μL of the cell-gel suspension is pipetted into the middle of each PDMS well that contains 175 micro-pillar arrays. A sterilized microscope slide is then used to evenly distribute the cell-gel solution throughout the micro-pillar array. Due to the prior plasma treatment of the PDMS plate, the cell-gel solution wicked into the micro-pillar arrays easily (Figure S3). After repeating this process for all micro-pillar arrays, the PDMS plate is incubated at 37° C./5.5% CO₂ for 25 min. Finally, each well is filled with supplemented DMEM high glucose media and a final concentration of 10 mM Y-27635.

### Bioinks:

The extracellular matrix (ECM) and media solutions were prepared.

First, a 15% (w/v) gelatin stock solution (solubilized at 90° C. for 12.5 h) is diluted to 5% gelatin (w/v) and combined with 15% fibrinogen (w/v).

Second, 1 mL of 10% gelbrin (w/v) solution is prepared in DMEM/F12 (Gibco). Third, DMEM High Glucose+10% FBS is supplemented with 50 μM Y-27635 (Biogems). Fourth, 100 U/mL thrombin solution is prepared in DMEM High Glucose+10% FBS. All ECM and media solutions were held at 37° C. using a hotplate. To chemically inhibit active compaction of the anisotropic OBBs and maintain their elongated structure, all aOBBs were incubated in the 50 μM solution of Y-27653 for 1 h at 37° C./5.5% CO₂. After 1 h, the aOBBs were harvested manually using a P1000 pipette with wide-bore tips. The wide-bore tips were treated with 3% bovine serum albumin (BSA) to prevent adsorption of the aOBBs. aOBBs were collected three wells at a time and transferred into a single 50 mL tube. Upon collection, the aOBBs were allowed to settle under gravity for 5 min. After the supernatant was aspirated, the volume (x mL) of compacted aOBBs was estimated. We subsequently wash compacted aOBBs in 3× volume of gelbrin bioink. After mixing with the wide-bore pipette, the 50 mL tube was centrifuged in 50 mL at 100 g for 3 min. Next, the gelbrin supernatant was aspirated and resuspended in another 1× volume of the gelbrin solution.

The aOBB laden bioink was transferred into a modified 1.0-cm³ glass syringe (Hamilton), which was pre-filled with ˜350 μL of the warmed 10% gelatin (w/v) and placed on ice for 1 min. Under ambient conditions, the bioink was loaded into the glass syringe through the drilled-out top followed by centrifugation for 1 min at 100 g. While the aOBBs were centrifuging, we attached a 1.5″ metal nozzle (Nordson EFD) to the aspirating line. After centrifugation, the supernatant was aspired and the remaining aOBB-laden bioink was added. This process was repeated until the bioink was fully loaded into the 1.0-cm³ glass syringe. The ink was then centrifuged for 5 min at 200 g and the residual supernatant was removed. The gelatin-fibrinogen ECM within the compacted aOBB-laden bioink was physically crosslinked by cooling in a 4° C. fridge for 15 min.

### Bioprinting of Aligned Cardiac Tissues:

A custom-designed two-part mold for a 3D printing platform was designed and fabricated, which facilitated the 3D printing process and enabled long-term culture of tissue. The two-part mold was printed using a stereolithography 3D printer (Perfactory Aureus, Envisiontec. Inc). The same photocurable resin (HTM140 V2, EnvisionTec) was used as was for the micro-pillar array fabrication. Design considerations included: (1) pillar caps and pillar ledges that maintain a consistent and reproducible z-height for the 3D printed filament, (2) internal shelfs in line with stability ledges for addition of sacrificial gelatin substrate, (3) reference caps that serve as zeroing location for the nozzle to initiate 3D printing, and (4) complementary pins and holes designed for a slip fit connection of the two-part molds. To prevent inhibition of platinum-based curing of silicones, the SLA-printed molds are plasma treated and silanized (see above, Micro-pillar Array Fabrication). To form a single mold, the two SLA-printed parts are tightly sealed by inserting an M4 nut through the side compression channels and tightening using wing-nuts. Upon assembly, soft silicone is poured into the mold (EcoFlex-050, SmoothOn) at a 1:1 mass ratio PartA:PartB. The silicone is polymerized at room temperature for >3 h. The platform is de-molded by hand by unscrewing the M4 threads and disassembling the two-part mold. This device is autoclaved prior to use.

All bioinks were dispensed using a custom-built syringe pump, which was controlled via an Arduino microcontroller and a stepper-motor driver. The syringe barrel was housed in a water-cooled, custom-built temperature controller. Before printing, the syringe was incubated in the temperature-controlled housing for >15 min. Immediately prior to printing, a pre-chilled, tapered plastic nozzle with an inner diameter of 0.61 mm (Nordson EFD) was fitted to the bottom of the syringe. The bioink was held at approximate 12° C., where it exhibits optimal printing behavior. The extrusion rate of the syringe pump was set to define a filament diameter below 1 mm at a print speed of 5 mm s⁻¹. After printing, the tissues were incubated in a 4° C. fridge for 5 min to ensure that the ECM within the bioink solidified. Next, cold DMEM high glucose supplemented with 10% FBS and 20 U/mL thrombin was added to the tissue and the entire printing platform was incubated at 37° C./5.5% CO₂. As the media increased in temperature, the thermally reversible gelatin in the bioink melted, which enabled the thrombin to simultaneously diffuse in and polymerize the fibrinogen into fibrin. After 1 h, the melted gelatin substrate was aspirated. Fresh DMEM high glucose+10% FBS is added and the tissue was returned to 37° C./5.5% CO₂.

### Cell Viability Assays:

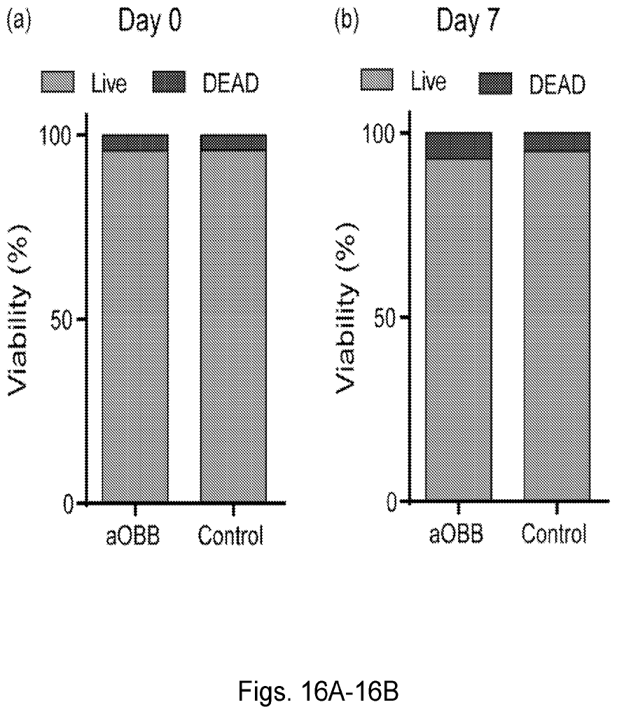

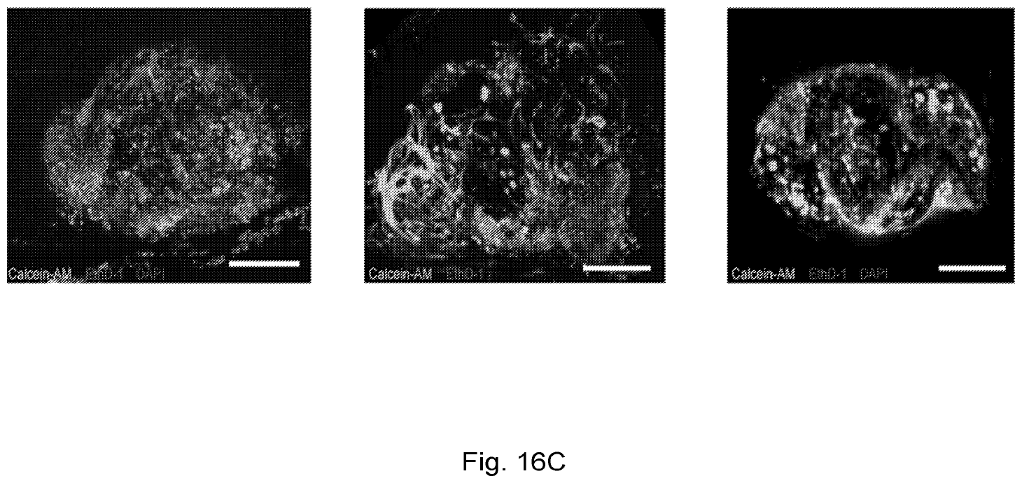

Bioprinted macro-tissues were sectioned and transferred via pipette into a 35-mm petri dish. The sectioned tissue was washed twice in standard PBS. Next, the tissue was incubated with Live/DEAD™ (ThermoFischer) solution (calcein-AM [2 μM], ethidium-homodimer-1 [4 μM]) and incubated at 37° C./5.5% CO₂ for 30 min. After incubation, Live/DEAD images were acquired on a confocal microscope (ZEISS). (FIGS. 16A-16C).

### Immunostaining and Flow Cytometry:

To prepare cardiac tissue for immunostaining, they were rinsed twice with standard PBS and fixed in a 4% paraformaldehyde (PFA) solution for 45 min at room temperature. After fixation, they were washed three times with PBS and blocked for 3 h in blocking solution (PBS, +5% (w/v) goat serum (GS), +0.25% (w/v) Triton-X). Following this incubation, blocking solution was removed and aOBBs were rinsed twice with standard PBS. Next, the aOBBs were incubated for 48 h at 4° C. in TNNT2/cTnT primary antibody (ab45932, Abeam) at 1:400 dilution in staining solution (PBS, +1% GS, +0.25% Triton-X). The aOBBs were then washed three times with PBST (0.1% Tween-20 in PBS) and left overnight at 4° C. The following day they were incubated with AlexaFlour-555 conjugated secondary antibody for 2 h. In the last 30 min of this incubation, Actin Green fluorescent probe (Invitrogen) was added according to manufacturer's instructions and 4′,6-diamidino-2-phenylindole (DAPI) nuclear stain was added at 1:3000 dilution. aOBBs were rinsed twice with PBST and remained in PBST at 4° C. before imaging.

Cardiac spheroids were prepared for flow cytometry analysis by first dissociating them into single cells, as described in prior work.⁴¹ Briefly, the spheroids were harvested on Day 11, washed in PBS, and incubated with CollagenaseB (1 mg/mL) for 1 h at 37° C./5.5% CO₂. Next, they were rinsed twice in cold DMEM/F12 and incubated with 25 U/mL of papain (Worthington, Inc.) in Earl's Balance Salt Solution (EBSS, +EDTA and L-cystein, Gibco) for 30 min at 37° C./5.5% CO₂. Papain was inhibited with DMEM/F12+10% FBS. Next, cells were rinsed with DMEM/F12 and pipetted several times with a P1000 pipette. The dissociated cells were rinsed twice in standard PBS. 1×10⁶ cells per condition were then incubated with fixable viability dye LIVE/DEAD Aqua (Thermofisher Scientific) at 1:2000 dilution for 15 min at room temperature (RT). All subsequent steps were performed in the dark. Next, cells were washed once with PBS and incubated for 15 min with BD Cytofix (BD Biosciences) at RT. After fixing, cells were washed in PBS (+3% BSA) three times and incubated in perm/wash buffer (Biolegend) for 30 min at RT. Following this incubation, cells were washed in perm/wash and incubated with conjugated antibodies: PE-Vimentin (562337, BD Biosciences) and Alexa Flour-647 cTnT (565744, BD Biosciences) for 30 min at 1:10 dilution in perm/wash at RT. Cells were next washed in perm/wash and then in cell staining buffer (Biolegend) and finally resuspended in PBS. Lastly, cell events were collected in a LSRII flow cytometer analyzer (BD Bioscience). Analysis is performed using FCS Express (De Novo Software,)

### Quantifying Tissue Alignment:

To quantify the alignment of aOBBs with the bioinks during printing, one must be able to identify, select, and measure their orientation. To identify individual aOBBs, all tissues were pre-incubated in three distinct fluorescent dyes with no or minimal overlapping emission spectra (CellTracker™ Green CMFDA [14.34 μM], CellTracker™ Orange CMRA [12.1 μM], CellTracker™ Deep Red [7.16 μM],). Fluorescent dyes were diluted in DMEM High Glucose (Corning) supplemented with 50 μM Y-27632 and incubated for 1 h at 37° C./5.5% CO₂. After incubation, three-dimensional images were acquired on a confocal microscope (ZEISS). The distribution of primary orientations angles (where 0° defines the direction of the print path) measured for each aOBB within the printed tissues were represented by radial histograms.

### Calcium Imaging:

A 5 μM Fluo-4 (Thermo Fischer Scientific) solution was prepared in Tyrode's solution (Alfa Aesar). The solution was supplemented to include a final concentration of 1 M Blebbistatin (Sigma-Aldrich) and 1 mM Probenicid (Thermo Fischer Scientific). Bioprinted cardiac tissues were incubated with the calcium indicator for 30 min at 37° C. Next, the tissues were rinsed 2× in Tyrodes supplemented with Blebbistatin. The samples were illuminated by a 488-nm light. Data was acquiring using Micro-Manager open-source software at 100 frames per second. In certain cases, these printed cardiac tissues were also electrically stimulated using platinum wires set 8 mm apart, inserted on either side of macro-filament. A mono-phase signal with a pulse of 2 ms was generated by custom-made Arduino-based controller at a frequency of 1-3 Hz and an amplitude of 10 V/cm.

### REFERENCES

1. Beladan, C. C., Călin, A., Roşca, M., Ginghină, C. & Popescu, B. A. Left ventricular twist dynamics: principles and applications. Heart 100, 731-740 (2014).
2. Triposkiadis, F. et al. Left ventricular geometry as a major determinant of left ventricular ejection fraction: physiological considerations and clinical implications. European Journal of Heart Failure 20, 436-444 (2018).
3. Sosnovik, D. E. & Tawakol, A. Noninvasive Tissue Characterization of Post-Infarction Myocardium. JACC: Cardiovascular Imaging (2018) doi:10.1016/j.jcmg.2017.11.040.
4. Sosnovik, D. E. et al. Microstructural impact of ischemia and bone marrow-derived cell therapy revealed with diffusion tensor magnetic resonance imaging tractography of the heart in vivo. Circulation (2014) doi:10.1161/CIRCULATIONAHA.113.005841.
5. Sosnovik, D. E. et al. Diffusion spectrum MRI tractography reveals the presence of a complex network of residual myofibers in infarcted myocardium. Circulation: Cardiovascular Imaging 2, 206-212 (2009).
6. Garcia-Canadilla, P. et al. Complex Congenital Heart Disease Associated With Disordered Myocardial Architecture in a Midtrimester Human Fetus. Circulation: Cardiovascular Imaging 11, (2018).
7. Weinberger, F., Mannhardt, I. & Eschenhagen. T. Engineering Cardiac Muscle Tissue. Circulation Research 120, 1487-1500 (2017).
8. Ogle, B. M. et al. Distilling complexity to advance cardiac tissue engineering. Science Translational Medicine 8, 342ps13-342ps13 (2016).
9. Tzatzalos, E., Abilez, 0. J., Shukla, P. & Wu, J. C. Engineered heart tissues and induced pluripotent stem cells: Macro- and microstructures for disease modeling, drug screening, and translational studies. Advanced Drug Delivery Reviews 96, 234-244 (2016).
10. Zhang, J., Zhu, W., Radisic, M. & Vunjak-Novakovic, G. Can We Engineer a Human Cardiac Patch for Therapy?Circulation Research 123, 244-265 (2018).
11. Bose, P., Eyckmans, J., Nguven, T. D., Chen, C. S. & Reich, D. H. Effects of Geometry on the Mechanics and Alignment of Three-Dimensional Engineered Microtissues. ACS Biomaterials Science and Engineering 5, 3843-3855 (2019).
12. Bursac, N., Parker, K. K., Iravanian, S. & Tung, L. Cardiomyocyte cultures with controlled macroscopic anisotropy: a model for functional electrophysiological studies of cardiac muscle. Circulation research 91, e45-e54 (2002).
13. Smith, A. S. T. et al. Micro- and nano-patterned conductive graphene-PEG hybrid scaffolds for cardiac tissue engineering. Chemical Communications 53, 7412-7415 (2017).
14. Wu, Y., Wang, L., Guo, B. & Ma, P. X. Interwoven Aligned Conductive Nanofiber Yarn/Hydrogel Composite Scaffolds for Engineered 3D Cardiac Anisotropy. ACS Nano (2017) doi:10.1021/acsnano.7b01062.
15. Wade, R. J., Bassin, E. J., Rodell, C. B. & Burdick, J. A. Protease-degradable electrospun fibrous hydrogels. Nature communications 6, 1-10 (2015).
16. Macqueen, L. A. et al. A tissue-engineered scale model of the heart ventricle. Nature Biomedical Engineering (2018) doi:10.1038/s41551-018-0271-5.
17. Schwan, J. et al. Anisotropic engineered heart tissue made from laser-cut decellularized myocardium. Scientific reports 6, 1-12 (2016).
18. Legant, W. R. et al. Microfabricated tissue gauges to measure and manipulate forces from 3D microtissues. Proceedings of the National Academy of Sciences of the United States of America (2009) doi:10.1073/pnas.0900174106.
19. Vandenburgh, H. et al. Drug-screening platform based on the contractility of tissue-engineered muscle. Muscle and Nerve 37, 438-447 (2008).
20. Grosberg, A., Alford, P. W., McCain, M. L. & Parker, K. K. Ensembles of engineered cardiac tissues for physiological and pharmacological study: Heart on a chip. Lab on a Chip 11, 4165 (2011).
21. Zimmermann, W. H. et al. Tissue engineering of a differentiated cardiac muscle construct. Circulation Research (2002) doi:10.1161/hh0202.103644.
22. Bian, W., Jackman, C. P. & Bursac, N. Controlling the structural and functional anisotropy of engineered cardiac tissues. Biofabrication 6, 024109 (2014).
23. Shadrin, I. Y. et al. Cardiopatch platform enables maturation and scale-up of human pluripotent stem cell-derived engineered heart tissues. Nature communications 8, 1-15 (2017).
24. Weinberger, F. et al. Cardiac repair in guinea pigs with human engineered heart tissue from induced pluripotent stem cells. Science translational medicine 8, 363ra148-363ra148 (2016).
25. Sydney Gladman, A., Matsumoto, E. A., Nuzzo, R. G., Mahadevan, L. & Lewis, J. A. Biomimetic 4D printing. Nature Materials 15, 413-418 (2016).
26. Compton, B. G. & Lewis, J. A. 3D-printing of lightweight cellular composites. Advanced Materials 26, 5930-5935 (2014).
27. Kotikian, A., Truby, R. L., Boley, J. W., White, T. J. & Lewis, J. A. 3D Printing of Liquid Crystal Elastomeric Actuators with Spatially Programed Nematic Order. Advanced Materials (2018) doi:10.1002/adma.201706164.
28. Mahajan, C. & Cormier, D. 3D printing of carbon fiber composites with preferentially aligned fibers. in IIE annual conference. Proceedings 2953 (Institute of Industrial and Systems Engineers (IISE), 2015).
29. Shofner, M. L., Lozano, K., Rodriguez-Macias, F. J. & Barrera, E. v. Nanofiber-reinforced polymers prepared by fused deposition modeling. Journal of applied polymer science 89, 3081-3090 (2003).
30. Spackman, C. C., Frank, C. R., Picha, K. C. & Samuel, J. 3D printing of fiber-reinforced soft composites: Process study and material characterization. Journal of Manufacturing Processes 23, 296-305 (2016).
31. Prendergast, M. E., Davidson, M. & Burdick, J. A. A biofabrication method to align cells within bioprinted photocrosslinkable and cell-degradable hydrogel constructs via embedded fibers. Biofabrication (2021).
32. Nerger, B. A., Brun, P.-T. & Nelson, C. M. Microextrusion printing cell-laden networks of type I collagen with patterned fiber alignment and geometry. Soft matter 15, 5728-5738 (2019).
33. Kim, H. et al. Shear-induced alignment of collagen fibrils using 3D cell printing for corneal stroma tissue engineering. Biofabrication 11, 35017 (2019).
34. Schwab, A. et al. Tissue mimetic hyaluronan bioink containing collagen fibers with controlled orientation modulating cell migration and alignment. Materials Today Bio 7, 100058 (2020).
35. Farkash, M. & Brandon, D. G. Whisker alignment by slip extrusion. Materials Science and Engineering: A 177, 269-275 (1994).
36. Costa, K. D., Lee, E. J. & Holmes, J. W. Creating alignment and anisotropy in engineered heart tissue: Role of boundary conditions in a model three-dimensional culture system. Tissue Engineering 9, 567-577 (2003).
37. Lee, E. J., Holmes, J. W. & Costa, K. D. Remodeling of engineered tissue anisotropy in response to altered loading conditions. Annals of biomedical engineering 36, 1322-1334 (2008).
38. Mukhija, D. & Solomon, M. J. Nematic order in suspensions of colloidal rods by application of a centrifugal field. Soft Matter 7, 540-545 (2011).
39. Zhao, M.-T., Ye, S., Su, J. & Garg, V. Cardiomyocyte proliferation and maturation: two sides of the same coin for heart regeneration. Frontiers in Cell and Developmental Biology 8, (2020).
40. Huebsch, N. et al. Miniaturized iPS-Cell-Derived Cardiac Muscles for Physiologically Relevant Drug Response Analyses. Scientific Reports 6, (2016).
41. Skylar-Scott, M. A. et al. Biomanufacturing of organ-specific tissues with high cellular density and embedded vascular channels. Science Advances 5, (2019).
42. Lian, X. et al. Directed cardiomyocyte differentiation from human pluripotent stem cells by modulating Wnt/β-catenin signaling under fully defined conditions. Nature protocols 8, 162-75(2012).

What is claimed herein is:

1. A method comprising applying a bioink material onto a support, the bioink material comprising:
a carrier fluid, and
a plurality of anisotropic organ building blocks (aOBBs), each of the plurality of aOBBs comprising:
at least one polymer; and
at least one cell;
wherein the applied bioink material comprises a plurality of aligned aOBBs.

2. The method of any one of the preceding claims, wherein at least 90% of the plurality of aligned aOBBs have an aOBB anisotropic axis that is within +/−60 degrees of a mean aoBB anisotropic axis of the plurality of aligned aoBBs.

3. The method of any one of the preceding claims, wherein at least 60% of the plurality of aligned aOBBs have an aOBB anisotropic axis that is within +/−30 degrees of a mean aoBB anisotropic axis of the plurality of aligned aoBBs.

4. The method of any one of the preceding claims, wherein at least 60% of the plurality of aligned aOBBs have an aOBB anisotropic axis that is within +/−10 degrees of a mean aoBB anisotropic axis of the plurality of aligned aoBBs.

5. The method of any one of the preceding claims, wherein at least 60% of the plurality of aligned aOBBs have an aOBB anisotropic axis that is within 1 standard deviation of a mean aoBB anisotropic axis of the plurality of aligned aoBBs.

6. The method of any one of the preceding claims, wherein at least 90% of the plurality of aligned aOBBs have an aOBB anisotropic axis that is within 2 standard deviations of a mean aoBB anisotropic axis of the plurality of aligned aoBBs.

7. The method of any one of the preceding claims, wherein at least 90% of the plurality of aligned aOBBs have an aOBB anisotropic axis that is within +/−60 degrees of an anisotropic axis of the applied bioink material.

8. The method of any one of the preceding claims, wherein at least 60% of the plurality of aligned aOBBs have an aOBB anisotropic axis that is within +/−30 degrees of an anisotropic axis of the applied bioink material.

9. The method of any one of the preceding claims, wherein at least 60% of the plurality of aligned aOBBs have an aOBB anisotropic axis that is within +/−10 degrees of an anisotropic axis of the applied bioink material.

10. The method of any one of the preceding claims, further comprising flowing the bioink material prior to or concurrently with the applying step.

11. The method of claim 10, wherein the flowing comprises exposing the bioink material to shear stress.

12. The method of any one of the preceding claims, wherein the applying comprises extruding, flowing, injecting the bioink material onto the support.

13. The method of any one of the preceding claims, wherein the applying comprises flowing the bioink material onto the support.

14. The method of any one of the preceding claims, wherein the applying comprises extruding the bioink material onto the support.

15. The method of any one of the preceding claims, wherein the applying comprises moving the bioink material through a nozzle, die, channel, or tube.

16. The method of any one of the preceding claims, wherein the applying comprises moving the bioink material through a nozzle.

17. The method of any one of the preceding claims, wherein the applied bioink material is in the form of one or more filaments.

18. The method of any one of the preceding claims, further comprising polymerizing the bioink material after the applying step.

19. The method of any one of the preceding claims, wherein the at least one polymer comprises at least one extracellular matrix material.

20. The method of any one of the preceding claims, wherein the at least one polymer comprises at least one synthetic polymer.

21. The method of any one of the preceding claims, wherein the at least one polymer comprises at least one biomaterial.

22. The method of any one of the preceding claims, wherein the at least one polymer comprises at least one material selected from the group consisting of:
collagen, fibrin, MATRIGEL, hyaluronic acid, silk, alginate, chitosan, alginate, gelatin, heparin, chondroitin sulfate, polyethylene glycol (PEG), polyethylene glycol diacrylate (PEGDA), polyglycolic acid PGA), poly(l)-lactic acid (PLA), poly(L) glycolate PLGA),and polyvinyl alcohol and their derivatives.

23. The method of any one of the preceding claims, wherein the at least one polymer comprises at least one material selected from the group consisting of:
fibrin, alginate, chitosan, alginate, gelatin, heparin, chondroitin sulfate, polyethylene glycol (PEG), and polyethylene glycol diacrylate (PEGDA).

24. The method of any one of the preceding claims, wherein the at least one polymer comprises or consists of collagen and fibrin.

25. The method of any one of the preceding claims, wherein the at least one polymer comprises or consists of alginate.

26. The method of any one of the preceding claims, wherein the at least one polymer comprises or consists of alginate and PEGDA; gelatin and alginate; gelatin and collagen; or gelatin and fibrin.

27. The method of any one of the preceding claims, wherein the at least one cell comprises functional cells, stromal cells, or a mixture of functional and stromal cells.

28. The method of any one of the preceding claims, wherein the at least one cell comprises cardiomyocytes and stromal cells.

29. The method of claim 28, wherein the cardiomyoctes comprise contractile induced pluripotent stem cell derived cardiomyocytes (iPSC-CMs), primary cardiomyocytes, and/or or embryonic stem cell derived cardiomyocytes (ESC-CM).

30. The method of any one of claims 27-29, wherein the stromal cells comprise fibroblasts.

31. The method of claim 30, wherein the fibroblasts comprise human neonatal dermal fibroblasts (hNDF).

32. The method of any one of the preceding claims, wherein the at least one cell further comprises one or more support cells selected from the group consisting of:
primary fibroblasts, mesenchymal stem cells, epicardial derived cells, and a combination thereof.

33. The method of any one of the preceding claims, wherein the at least one cell comprises one or more of:
contractile skeletal muscle cells (either primary or stem cell derived), mesenchymal stem cells (e.g., stromal cells), neurons, endothelial cells, smooth-muscle cells, and fibroblasts.

34. The method of any one of the preceding claims, wherein the at least one cell is a plurality of cells.

35. The method of claim 34, wherein the plurality of cells of an aOBB are cellularly aligned.

36. The method of claim 35, wherein at least 90% of the plurality of cells have a cellular anisotropic axis that is within +/−60 degrees of a mean cellular anisotropic axis of the plurality of cells of the aOBB.

37. The method of any one of claims 34-36, wherein at least 60% of the plurality of cells have a cellular anisotropic axis that is within +/−30 degrees of a mean cellular anisotropic axis of the plurality of cells of the aOBB.

38. The method of any one of claims 34-37, wherein at least 60% of the plurality of cells have a cellular anisotropic axis that is within +/−10 degrees of a mean cellular anisotropic axis of the plurality of cells of the aOBB.

39. The method of any one of claims 34-38, wherein at least 60% of the plurality of cells have a cellular anisotropic axis that is within 1 standard deviation of a mean cellular anisotropic axis of the plurality of cells of the aOBB.

40. The method of any one of claims 34-39, wherein at least 90% of the plurality of cells have a cellular anisotropic axis that is within 2 standard deviations of a mean cellular anisotropic axis of the plurality of cells of the aOBB.

41. The method of any one of the preceding claims, wherein the bioink material comprises 10-1000e6 cells/mL.

42. The method of any one of the preceding claims, wherein the plurality of aOBBs are at least 5% volume of the bioink material.

43. The method of any one of the preceding claims, wherein the carrier fluid comprises one or more of processed gelatin, fibrinogen, collagen, alginate, RGD-modified alginate, fibrin, and cell media.

44. The method of any one of the preceding claims, wherein the carrier fluid comprises one or more of processed gelatin, fibrinogen, and cell media.

45. The method of any one of the preceding claims, further comprising compacting the bioink material before the applying step.

46. The method of claim 45, wherein the compacting comprises centrifugation, capillary flow to increase the aOBB density in the bioink material, or evaporation.

47. The method of any one of the preceding claims, wherein the support is a glass support, a plastic support, or a biological hydrogel or matrix.

48. The method of any one of the preceding claims, wherein the support does not comprise an anisotropic scaffold.

49. The method of any one of the preceding claims, further comprising a first step of providing the bioink material, comprising:
culturing the aOBBs;
contacting the aOBBs with the carrier fluid; and
compacting the aOBBs.

50. The method of any one of the preceding claims, further comprising a first step of providing the bioink material, comprising:
culturing the aOBBs within a pillar array;
contacting the aOBBs with at least one ROCK inhibitor;
harvesting the aOBBs from the pillar array by manual pipetting or by degradation of the pillar array;
resuspending the aOBBs in the carrier fluid; and
compacting the aOBBs.

51. The method of any one of the preceding claims, further comprising a first step of providing the bioink material, comprising:
culturing the aOBBs within a pillar array;
harvesting the aOBBs from the pillar array by manual pipetting or by degradation of the pillar array;
resuspending the aOBBs in the carrier fluid;
compacting the aOBBs; and
adjusting rheology of the bioink material by modulating temperature.

52. The method of any one of the preceding claims, wherein the applied bioink forms a pattern(s) selected from the group consisting of linear, chevron, spiral pattern, and a combination of patterns.

53. The method of any one of the preceding claims, further comprising culturing the applied bioink to form a tissue or tissue construct.

54. The method of claim 53, further comprising vascularizing the tissue or tissue construct.

55. A tissue or tissue construct prepared by the method of any of claims 1 to 54 aligned at the aOBB, cellular, and sarcomeric length scales.

56. The tissue or tissue construct of claim 55, wherein the aOBBS have isotropic cellular alignment.

57. The tissue or tissue construct of claim 55, wherein the aOBBs have anisotropic cellular alignment.

58. Use of a tissue or tissue construct produced by the methods of any of claims 1 to 54 in food production.

59. Use of a tissue or tissue construct produced by the methods of any of claims 1 to 54 in disease modeling.

60. Use of a tissue or tissue construct produced by the methods of any of claims 1 to 54 in cardiac disease modeling.

61. Use of a tissue or tissue construct produced by the methods of any of claims 1 to 54 in drug toxicity studies.

62. Use of a tissue or tissue construct produced by the methods of any of claims 1 to 54 in drug screening applications.

63. Use of a tissue or tissue construct produced by the methods of any of claims 1 to 54 as cardiac tissue for replacement of heart in regenerative medicine.

64. A bioink material comprising:
a carrier fluid, and
a plurality of anisotropic organ building blocks (aOBBs), each of the plurality of aOBBs comprising:
at least one polymer; and
at least one cell;
wherein at least some of the aOBBs are capable of anisotropically aligning along a flow direction when the bioink is flowed or is extruded from a three-dimensional printer or additive manufacturing system.

65. The bioink of claim 64, wherein the cellular density within the bioink is 10-500e6 cell/mL.

66. The bioink of any one of claims 64-65, wherein at least 90% of the plurality of aligned aOBBs have an aOBB anisotropic axis that is within +/−60 degrees of a mean aoBB anisotropic axis of the plurality of aligned aoBBs.

67. The bioink of any one of claims 64-66, wherein at least 60% of the plurality of aligned aOBBs have an aOBB anisotropic axis that is within +/−30 degrees of a mean aoBB anisotropic axis of the plurality of aligned aoBBs.

68. The bioink of any one of claims 64-67, wherein at least 60% of the plurality of aligned aOBBs have an aOBB anisotropic axis that is within +/−10 degrees of a mean aoBB anisotropic axis of the plurality of aligned aoBBs.

69. The bioink of any one of claims 64-68, wherein at least 60% of the plurality of aligned aOBBs have an aOBB anisotropic axis that is within 1 standard deviation of a mean aoBB anisotropic axis of the plurality of aligned aoBBs.

70. The bioink of any one of claims 64-69, wherein at least 90% of the plurality of aligned aOBBs have an aOBB anisotropic axis that is within 2 standard deviations of a mean aoBB anisotropic axis of the plurality of aligned aoBBs.

71. The bioink of any one of claims 64-70, wherein at least 90% of the plurality of aligned aOBBs have an aOBB anisotropic axis that is within +/−60 degrees of an anisotropic axis of the applied bioink material.

72. The bioink of any one of claims 64-71, wherein at least 60% of the plurality of aligned aOBBs have an aOBB anisotropic axis that is within +/−30 degrees of an anisotropic axis of the applied bioink material.

73. The bioink of any one of claims 64-72, wherein at least 60% of the plurality of aligned aOBBs have an aOBB anisotropic axis that is within +/−10 degrees of an anisotropic axis of the applied bioink material.

74. The bioink of any one of claims 64-73, wherein the at least one polymer comprises at least one extracellular matrix material.

75. The bioink of any one of claims 64-74, wherein the at least one polymer comprises at least one synthetic polymer.

76. The bioink of any one of claims 64-75, wherein the at least one polymer comprises at least one biomaterial.

77. The bioink of any one of claims 64-76, wherein the at least one polymer comprises at least one material selected from the group consisting of:
collagen, fibrin, MATRIGEL, hyaluronic acid, silk, alginate, chitosan, alginate, gelatin, heparin, chondroitin sulfate, polyethylene glycol (PEG), polyethylene glycol diacrylate (PEGDA), polyglycolic acid PGA), poly(l)-lactic acid (PLA), poly(L) glycolate PLGA),and polyvinyl alcohol and their derivatives.

78. The bioink of any one of claims 64-77, wherein the at least one polymer comprises at least one material selected from the group consisting of:
fibrin, alginate, chitosan, alginate, gelatin, heparin, chondroitin sulfate, polyethylene glycol (PEG), and polyethylene glycol diacrylate (PEGDA).

79. The bioink of any one of claims 64-78, wherein the at least one polymer comprises or consists of collagen and fibrin.

80. The bioink of any one of claims 64-79, wherein the at least one polymer comprises or consists of alginate.

81. The bioink of any one of claims 64-80, wherein the at least one polymer comprises or consists of alginate and PEGDA; gelatin and alginate; gelatin and collagen; or gelatin and fibrin.

82. The bioink of any one of claims 64-81, wherein the at least one cell comprises functional cells, stromal cells, or a mixture of functional and stromal cells.

83. The bioink of any one of claims 64-82, wherein the at least one cell comprises cardiomyocytes and stromal cells.

84. The bioink of any claim 83, wherein the cardiomyoctes comprise contractile induced pluripotent stem cell derived cardiomyocytes (iPSC-CMs), primary cardiomyocytes, and/or or embryonic stem cell derived cardiomyocytes (ESC-CM).

85. The bioink of any one of claims 64-84, wherein the stromal cells comprise fibroblasts.

86. The bioink of claim 85, wherein the fibroblasts comprise human neonatal dermal fibroblasts (hNDF).

87. The bioink of any one of claims 64-86, wherein the at least one cell further comprises one or more support cells selected from the group consisting of:
primary fibroblasts, mesenchymal stem cells, epicardial derived cells, and a combination thereof.

88. The bioink of any one of claims 64-87, wherein the at least one cell comprises one or more of:
contractile skeletal muscle cells (either primary or stem cell derived), mesenchymal stem cells (e.g., stromal cells), neurons, endothelial cells, smooth-muscle cells, and fibroblasts.

89. The bioink of any one of claims 64-88, wherein the at least one cell is a plurality of cells.

90. The bioink of claim 89, wherein the plurality of cells of an aOBB are cellularly aligned.

91. The bioink of claim 90, wherein at least 90% of the plurality of cells have a cellular anisotropic axis that is within +/−60 degrees of a mean cellular anisotropic axis of the plurality of cells of the aOBB.

92. The bioink of any one of claims 90-91, wherein at least 60% of the plurality of cells have a cellular anisotropic axis that is within +/−30 degrees of a mean cellular anisotropic axis of the plurality of cells of the aOBB.

93. The bioink of any one of claims 90-92, wherein at least 60% of the plurality of cells have a cellular anisotropic axis that is within +/−10 degrees of a mean cellular anisotropic axis of the plurality of cells of the aOBB.

94. The bioink of any one of claims 90-93, wherein at least 60% of the plurality of cells have a cellular anisotropic axis that is within 1 standard deviation of a mean cellular anisotropic axis of the plurality of cells of the aOBB.

95. The bioink of any one of claims 90-94, wherein at least 90% of the plurality of cells have a cellular anisotropic axis that is within 2 standard deviations of a mean cellular anisotropic axis of the plurality of cells of the aOBB.

96. The bioink of any one of claims 64-95, wherein the plurality of aOBBs are at least 5% volume of the bioink material.

97. The bioink of any one of claims 64-96, wherein the carrier fluid comprises one or more of processed gelatin, fibrinogen, collagen, alginate, RGD-modified alginate, fibrin, and cell media.

98. The bioink of any one of claims 64-97, wherein the carrier fluid comprises one or more of processed gelatin, fibrinogen, and cell media.
